SEQUENCE LISTING (23 sequences)

SEQ ID NO: 1 - protein, 11 aa, Homo sapiens
RASQSVSSAVA

SEQ ID NO: 2 - protein, 7 aa, Homo sapiens
SASSLYS

SEQ ID NO: 3 - protein, 9 aa, Homo sapiens
QQSSTDPIT

SEQ ID NO: 4 - protein, 9 aa, Homo sapiens
FTVSSSSIH

SEQ ID NO: 5 - protein, 17 aa, Homo sapiens
YISSSSGYTYYADSVKG

SEQ ID NO: 6 - protein, 15 aa, Homo sapiens
YWSYPSWWPYRGMDY

SEQ ID NO: 7 - protein, 124 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, Homo sapiens
MYRMQLLSCIALSLALVTNS

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2022/0185896 A1
(43) Pub. Date: Jun. 16, 2022

(54) ANTI-GALECTIN-9 ANTIBODIES AND USES THEREOF

(71) Applicants: New York University, New York, NY (US); PureTech LYT, Inc., Boston, MA (US)

(72) Inventors: Shohei KOIDE, New York, NY (US); George MILLER, New York, NY (US); Akiko KOIDE, New York, NY (US); Linxiao CHEN, New York, NY (US); Aleksandra FILIPOVIC, London (GB); Eric ELENKO, Boston, MA (US); Joseph BOLEN, Boston, MA (US)

(21) Appl. No.: 17/607,878

(22) PCT Filed: May 1, 2020
(86) PCT No.: PCT/US2020/031181

§ 371 Date: Oct. 30, 2021

Related U.S. Application Data
(60) Provisional application No. 62/841,732, filed on May 1, 2019.

(51) Int. Cl. C07K 16/28; A61P 35/04; A61P 35/00

(52) U.S. Cl. CPC C07K 16/2851; C07K 16/2818; A61K 2039/545; A61P 35/00; A61P 35/04

(57) ABSTRACT

Disclosed herein are methods for treating solid tumors (e.g., pancreatic adenocarcinoma (PDA), colorectal cancer (CRC), hepatocellular carcinoma (HCC)), or Cholangiocarcinoma and others), including, but not limited to, metastatic tumors, using an anti-Galectin-9 antibody.

### RELATED APPLICATION

This application claims the benefit of U.S. Provisional Application No. 62/841,732, filed on May 1, 2019, which is incorporated by reference herein in its entirety.

### BACKGROUND OF INVENTION

The immune system holds remarkable potential to recognize and destroy cancer cells, but the complex network governing tumor immune escape is an obstacle to broadly effective immune modulation (Martinez-Bosch N, et al., Immune Evasion in Pancreatic Cancer: From Mechanisms to Therapy. Cancers (Basel). 2018; 10 (1)). Approved immuno-oncology (IO) agents deliver incremental survival improvements to many tumor types (e.g. melanoma, lung, renal, bladder cancer, some colon cancers etc.), and are being rapidly integrated as standard of care in addition to and in conjunction with surgery, chemotherapy, and radiotherapy. However, there is still a major gap in the treatment and survivorship of multiple other aggressive malignancies. For example, metastatic pancreatic ductal adenocarcinoma (PDAC or PDA), cholangiocarcinoma (CCA) and colorectal cancer (CRC) still have 5-year survival rates of <9%, <5% and <15%, respectively. These gastrointestinal tumors are very aggressive, many patients have advanced-stage disease at presentation, and the effectiveness of approved immunotherapies is suboptimal (Rizvi, et al., Cholangiocarcinoma—evolving concepts and therapeutic strategies; Nat Rev Clin Oncol. 2018; 15(2):95-111; Kalyan, et al., Updates on immunotherapy for colorectal cancer; J Gastrointest Oncol. 2018; 9(1):160-169).

The success of first generation checkpoint inhibitors (anti-PD1, anti-PDL1, and anti-CTLA4) has led to an explosion of new IO clinical trial efficacy and differentiation (Holl et al., Examining Peripheral and Tumor Cellular Immunome in Patients With Cancer; Front Immunol. 2019; 10:1767). However, among successes, there have also been many unfortunate development failures, consequently, there is still a need for more novel and efficacious treatments.

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist, and can be located within the cell or extracellularly. Elevated Galectin-9 levels have been in observed a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, Galectin-9 was expressed in 57% of tumors and was significantly increased in the plasma of patients with advanced melanoma compared to healthy controls (Enninga et al., Melanoma Res. 2016 October; 26(5): 429-441). A number of studies have shown utility for Galectin-9 as a prognostic marker, and more recently as a potential new drug target (Enninga et al., 2016; Kawashima et al. BJU Int 2014; 113: 320-332; Kageshita et al., Int J Cancer. Jun. 20, 2002; 99(6):809-16, and references therein).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDA), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

### SUMMARY OF INVENTION

The present disclosure is based, at least in part, on the development of treatment regimen for solid tumors (e.g., metastatic solid tumors) such as pancreatic ductal adenocarcinoma (PDAC), colorectal cancer (CRC), hepatocellular carcinoma (HCC), and cholangiocarcinoma (CAA), involving an antibody capable of binding to human Galectin-9, either alone or in combination with a checkpoint inhibitor such as an anti-PD-1 antibody.

Accordingly, one aspect of the present disclosure provides a method for treating a solid tumor in a subject by administering an antibody that binds human Galectin-9. In some embodiments, the solid tumor is pancreatic adenocarcinoma (PDA), colorectal cancer (CRC), or hepatocellular carcinoma (HCC), or cholangiocarcinoma. In some embodiments, the method comprises administering to a subject having a solid tumor, e.g., PDA, CRC, HCC, or CCA an effective amount of an antibody that binds human Galectin-9 (referred to herein as an anti-Gal 9 antibody or anti-Galectin-9 antibody).

In some embodiments, the anti-Galectin-9 antibody is antibody G9.2-17, the structure of which is provided herein. In some embodiments, the anti-Galectin-9 antibody comprises the same heavy chain complementary determining regions (CDRs) and/or the same light chain CDRs as reference antibody G9.2-17, the sequences of which are provided herein. In some embodiments, the anti-Galectin-9 antibody comprises the heavy chain variable domain of antibody G9.2-17, and/or a light chain variable domain of antibody G9.2-17.

In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4.

In some embodiments, the anti-Galectin-9 antibody is administered to the subject at a dose of about 1 mg/kg to about 30 mg/kg (e.g., about 3 mg/kg to about 15 mg/kg or about 2 mg/kg to about 16 mg/kg) once every 2-3 weeks. In some embodiments, the anti-Galectin-9 antibody is administered to the subject at a dose selected from 2 mg/kg, 4 mg/kg, 8 mg/kg, 12 mg/kg, or 16 mg/kg. In some embodiments, the antibody is administered once every 2 weeks. In some embodiments, the anti-Galectin-9 antibody is administered to the subject at a dose selected from 2 mg/kg, 4 mg/kg, 8 mg/kg, 12 mg/kg, or 16 mg/kg once every 2 weeks. In some embodiments, the anti-Galectin-9 antibody is administered once every 2 weeks for one cycle, once every 2 weeks for two cycles, once every 2 weeks for 3 cycles, once every 2 weeks for 4 cycles, or once every 2 weeks for more than 4 cycles. In some embodiments, the duration of treatment is 0-3 months, 3-6 months, 12-24 months or longer. In some embodiments, the duration of treatment is 12-24 months or longer. In some embodiments, the cycles extend for a duration of 3 months to 6 months, or 6 months to 12 months or 12 months to 24 months or longer. In some embodiments, the cycle length is modified, e.g., temporarily or permanently to a longer duration, e.g., 3 weeks or 4 weeks. In some embodiments, the anti-Galectin-9 antibody is administered to the subject by intravenous infusion. In some embodiments, the cancer is metastatic cancer, including a metastatic cancer of any of the above mentioned cancers. In some embodiments, the method of treatment comprising administering the anti-Galectin-9 antibody does not include any other concurrent anti-cancer therapy.

In some embodiments, the method of treatment employing the anti-Galectin-9 antibody includes another concurrent anti-cancer therapy. Thus, in some embodiments, the method of treatment employing the anti-Galectin-9 antibody further comprises administering to the subject an immune checkpoint inhibitor. In some embodiments, the immune checkpoint inhibitor is an antibody that binds PD-1, for example, pembrolizumab, nivolumab, tislelizumab, orcemiplimab. In some embodiments, the antibody that binds PD-1 is nivolumab, which is administered to the subject at a dose of 240 mg once every two weeks. In some embodiments, the antibody that binds PD-1 is nivolumab, which is administered to the subject at a dose of 480 mg once every 4 weeks. In some embodiments, the antibody that binds PD-1 is prembrolizumab, which is administered at a dose of 200 mg once every 3 weeks. In some embodiments, the antibody that binds PD-1 is cemiplimab, which is administered at a dose of 350 mg once every 3 weeks. In some embodiments, the antibody that binds PD-1 is Tislelizumab, which is administered at a dose of 200 mg once every 3 weeks. In some embodiments, the immune checkpoint inhibitor is administered by intravenous infusion.

In some embodiments, the anti-Galectin-9 antibody comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6 and/or comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3. In some embodiments, the anti-Galectin-9 antibody comprises a heavy chain variable domain of SEQ ID NO: 7, and/or a light chain variable domain of SEQ ID NO: 8. In some embodiments, the anti-Galectin-9 antibody is a full-length antibody. In some embodiments, the anti-Galectin-9 antibody is an IgG1 or IgG4 molecule. In some embodiments, the anti-Galectin-9 antibody is a human IgG4 molecule having a modified Fc region of human IgG4. In some embodiments, the modified Fc region of human IgG4 comprises the amino acid sequence of SEQ ID NO: 14. In some embodiments, the modified Fc region of human IgG4 comprises the amino acid sequence of SEQ ID NO: 21. In some embodiments, the anti-Galectin-9 antibody comprises a heavy chain comprising the amino acid sequence of SEQ ID NO: 19 and a light chain comprising the amino acid sequence of SEQ ID NO: 15. In some embodiments, the anti-Galectin-9 antibody comprises a heavy chain comprising the amino acid sequence of SEQ ID NO: 23 and a light chain comprising the amino acid sequence of SEQ ID NO: 15.

In any of the methods disclosed herein, the subject (e.g., a human patient) may have undergone one or more prior anti-cancer therapies, e.g., surgery, chemotherapy, immunotherapy, radiation therapy, a therapy involving a biologic, targeted small molecule, hormonal agent, or a combination thereof. In some embodiments, the subject has progressed disease through the one or more prior anti-cancer therapies. In other embodiments, the subject is resistant (e.g., de novo, or acquired) to the one or more prior therapies. In other embodiments, the subject has relapsed after one or more prior therapies.

In any of the treatment methods disclosed herein, the subject can be a human patient having an elevated level of Galectin-9 relative to a control value. In some embodiments, the human patient has an elevated serum or plasma level of Galectin-9 relative to the control value. In some embodiments, the human patient has an elevated level of Galectin-9 expressed on the surface of cells derived from the human patient as relative to the control value. Such cells can be cancer cells and/or immune cells in the tumor and/or in the blood of a cancer patient. In some examples, the cancer cells are in tumor organoids derived from the human patient. In some embodiments, the control value is based on a value obtained from a healthy human subject.

Any of the treatment methods disclosed herein may further comprise monitoring occurrence of adverse effects in the subject. In case adverse effects (e.g., one or more severe adverse effects occur), either the dose of the anti-Galectin-9 antibody (e.g., G9.2-17), or the dose of the checkpoint inhibitor if co-used (e.g., the anti-PD-1 antibody such as nivolumab), or both may be reduced.

Also within the scope of the present disclosure are pharmaceutical compositions for use in treating a solid tumor (e.g., those described herein and including metastatic solid tumors), and uses of any of the anti-Galectin-9 antibodies for manufacturing a medicament for treating the solid tumor, wherein the uses disclosed herein, in some embodiments, involve one or more of the treatment conditions (e.g., dose, dosing regimen, administration route, etc.) as also disclosed herein.

The details of one or more embodiments of the invention are set forth in the description below. Other features or advantages of the present invention are be apparent from the following drawing and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to the drawing in combination with the detailed description of specific embodiments presented herein.

FIG. 1 is a graph showing a representative size exclusion chromatography (SEC) profile for the anti-Galectin-9 antibody. The high molecular weight peaks are labeled.

FIGS. 2A-2F include bar graphs showing levels of Galectin-9 expression as measured in T cells (CD3⁺), macrophages (CD11b+) and tumor cells (Epcam+) in S2 and S3 organoid fractions derived from a pancreatic adenocarcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and “fluorescence minus one” (FMO) 9M1-3 (“Gal9 FMO”) were used as controls for specificity, background staining and fluorescence bleed through from other channels. FIG. 2A shows levels of Galectin-9 in CD3⁺ cells as measured in the S3 fraction. FIG. 2B shows levels of Galectin-9 in CD11b⁺ cells as measured in the S3 fraction. FIG. 2C shows levels of Galectin-9 in Epcam⁺ cells as measured in the S3 fraction. FIG. 2D shows levels of Galectin-9 in CD3⁺ cells as measured in the S2 fraction. FIG. 2E shows levels of Galectin-9 in CD11b⁺ cells as measured in the S2 fraction. FIG. 2F shows levels of Galectin-9 in Epcam⁺ cells as measured in the S2 fraction.

FIGS. 3A-3F include bar graphs showing levels of Galectin-9 expression as measured in T cells (CD3⁺), macrophages (CD11b+) and tumor cells (Epcam+) in S2 and S3 organoid fractions derived from a colorectal carcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used controls for specificity, background staining and fluorescence bleed through from other channels. FIG. 3A shows levels of Galectin-9 in CD3⁺ cells as measured in the S3 fraction. FIG. 3B shows levels of Galectin-9 in CD11b⁺ cells as measured in the S3 fraction. FIG. 3C shows levels of Galectin-9 in Epcam⁺ cells as measured in the S3 fraction. FIG. 3D shows levels of Galectin-9 in CD3⁺ cells as measured in the S2 fraction. FIG. 3E shows levels of Galectin-9 in CD11b⁺ cells as measured in the S2 fraction. FIG. 3F shows levels of Galectin-9 in Epcam⁺ cells as measured in the S2 fraction.

FIGS. 4A-4F include bar graphs showing levels of Galectin-9 expression as measured in T cells (CD3⁺), macrophages (CD11b+) and tumor cells (Epcam+) in S2 and S3 organoid fractions derived from a second pancreatic adenocarcinoma biopsy using anti-Galectin-9 G9.2-17 Fab fragment and a commercially available Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used as controls for specificity, background staining and fluorescence bleed through from other channels. FIG. 4A shows levels of Galectin-9 in CD3⁺ cells as measured in the S3 fraction. FIG. 4B shows levels of Galectin-9 in CD11b⁺ cells as measured in the S3 fraction. FIG. 4C shows levels of Galectin-9 in Epcam+ cells as measured in the S3 fraction. FIG. 4D shows levels of Galectin-9 in CD3⁺ cells as measured in the S2 fraction. FIG. 4E shows levels of Galectin-9 in CD11b⁺ cells as measured in the S2 fraction. FIG. 4F shows levels of Galectin-9 in Epcam⁺ cells as measured in the S2 fraction.

FIGS. 5A-5C include photographs of immunohistochemical analysis of various tumors using anti-Galectin-9 antibody 1G3. All magnifications are 200×. FIG. 5A shows chemotherapy-treated colorectal cancer with heterogeneous intensity score 2 and 3 (moderate and high) Galectin-9 expression. Galectin-9 staining was observed at the cell membrane in particular; additionally, intraglandular macrophages are moderately positive and stromal reaction in tumor shows multinucleated macrophage giant cells with moderately strong Galectin-9 expression. FIG. 5B shows liver metastasis of colorectal carcinoma with high (intensity score 3) Galectin-9 expression. Staining is located on the membrane and in the cytoplasm. FIG. 5C shows Galectin-9 positive (intensity score 2) entrapped bile ducts and Galectin-9 negative cancer.

FIG. 6 includes a graph showing the fraction of annexin V- and propidium iodide (PI)-positive cells plotted as a function of antibody concentration used. MOLM-13 cells were co-incubated with varying concentrations of either G9.2-17 or human IgG4 isotype antibody and recombinant human Galectin-9 for 16 hours. Cells were stained with annexin V and propidium iodide prior to analysis by flow cytometry. Each condition was performed in triplicate. Analysis was performed on FlowJo software.

FIGS. 7A and 7B depict graphs showing results of a study in which mice treated with G9.2-17 mIgG2a alone or in combination with αPD1 mAb. Mice (n=10/group) with orthotopically implanted KPC tumors were treated with commercial αPD-1 (200 μg) mAb or G9.2-17 mIg2a (200 μg), or a combination of G9.2-17 and αPD-1, or matched isotype once weekly for three weeks. Tumors were removed and weighed (FIG. 7A) and subsequently processed and stained for flow cytometry (FIG. 7B). Each point represents one mouse; *p<0.05; **p<0.01; ***p<0.001; ****p<0.0001; by unpaired Student's t-test.

FIG. 7B depicts bar graphs showing tumors were excised from control and treated animals at the end of experiment (Day 18) and processed for flow cytometry of intra-tumoral immune cells and related activation and immunosuppressive markers. Mouse tumors were digested before flow. Flow cytometry was carried out on the Attune NxT flow cytometer (ThermoFisher Scientific, Waltham, Mass.). Data were analyzed using FlowJo v.10.1 (Treestar, Ashland, Oreg.)

FIGS. 8A and 8B depict graphs showing the results of ADCC assays performed with the IgG1 form of G9.2-17 (FIG. 8A) and the IgG4 form of G9.2-17 (FIG. 8B). As expected for a human IgG4 mAb, G9.2-17 does not mediate ADCC (FIG. 8B). This was tested against the IgG1 human counterpart of G9.2-17 as a positive control, which mediates ADCC and ADCP, as expected (FIG. 8A).

FIGS. 9A and 9B depict graphs showing the effect of 9.2-17 in a B16F10 subcutaneous syngeneic model. Tumors were engrafted subcutaneously and treated with G9.2-17 IgG1 mouse mAb, anti-PD1 antibody or a combination of G9.2-17 IgG1 mouse mAb and anti-PD1 antibody. FIG. 9A depicts a graph showing the effect on tumor volume. FIG. 9B depicts a graph showing intratumoral CD8 T cell infiltration. Results show that intra-tumoral presence effector T cells were enhanced in the combination arm.

FIGS. 10A and 10B include charts showing cholangiocarcinoma patient-derived tumor cultures ex vivo (organoids) treated with G9.2-17. Patient derived tumor cultures ex vivo (organoids) were treated with G9.2-17 or isotype control for three days. Expression of CD44 (FIG. 10A), and TNFα (FIG. 10B) in CD3+ T cells from PDOTS was assessed.

### DETAILED DESCRIPTION OF INVENTION

Provided herein are methods of using anti-Galectin-9 antibodies, e.g., G9.2-17, for treating solid tumors, for example, pancreatic adenocarcinoma (PDA), colorectal cancer (CRC), hepatocellular carcinoma (HCC), and cholangiocarcinoma. In some embodiments, the cancers are metastatic. In some embodiments, the methods disclosed herein provide specific doses and/or dosing schedules. In some instances, the methods disclosed herein target specific patient populations, for example, patients who have undergone prior treatment and show disease progression through the prior treatment, or patients who are resistant (de novo or acquired) to the prior treatment.

Galectin-9, a tandem-repeat lectin, is a beta-galactoside-binding protein, which has been shown to have a role in modulating cell-cell and cell-matrix interactions. It is found to be strongly overexpressed in Hodgkin's disease tissue and in other pathologic states. It has in some instances also been found circulating in the tumor microenvironment (TME).

Galectin-9 is found to interact with Dectin-1, an innate immune receptor which is highly expressed on macrophages in PDA, as well as on cancer cells (Daley, et al. Nat Med. 2017; 23(5):556-6). Regardless of the source of Galectin-9, disruption of its interaction with Dectin-1 has been shown to lead to the reprogramming of CD4⁺ and CD8⁺ cells into indispensable mediators of anti-tumor immunity. Thus, Galectin-9 serves as a valuable therapeutic target for blocking the signaling mediated by Dectin-1. Accordingly, in some embodiments, the anti-Galectin-9 antibodies describe herein disrupt the interaction between Galectin-9 and Dectin-1.

Galectin-9 is also found to interact with TIM-3, a type I cell surface glycoprotein expressed on the surface of leukemic stem cells in all varieties of acute myeloid leukemia (except for M3 (acute promyelocytic leukemia)), but not expressed in normal human hematopoietic stem cells (HSCs). TIM-3 signaling resulting from Galectin-9 ligation has been found to have a pleiotropic effect on immune cells, inducing apoptosis in Th1 cells (Zhu et al., Nat Immunol., 2005, 6:1245-1252) and stimulating the secretion of tumor necrosis factor-α (TNF-α), leading to the maturation of monocytes into dendritic cells, resulting in inflammation by innate immunity (Kuchroo et al., Nat Rev Immunol., 2008, 8:577-580). Further Galectin-9/TIM-3 signaling has been found to co-activate NF-κB and β-catenin signaling, two pathways that promote LSC self-renewal (Kikushige et al., Cell Stem Cell, 2015, 17(3):341-352). An anti-Galectin-9 antibody that interferes with Galectin-9/TIM-3 binding could have a therapeutic effect, especially with respect to leukemia and other hematological malignancies. Accordingly, in some embodiments, the anti-Galectin-9 antibodies described herein disrupt the interaction between Galectin-9 and TIM-3.

Further, Galectin-9 is found to interact with CD206, a mannose receptor highly expressed on M2 polarized macrophages, thereby promoting tumor survival (Enninga et al., J Pathol. 2018 August; 245(4):468-477). Tumor-associated macrophages expressing CD206 are mediators of tumor immunosuppression, angiogenesis, metastasis, and relapse (see, e.g., Scodeller et al., Sci Rep. Nov. 7, 2017; 7(1):14655, and references therein). Specifically, M1 (also termed classically activated macrophages) are trigged by Th1-related cytokines and bacterial products, express high levels of IL-12, and are tumoricidal. By contrast, M2 (so-called alternatively activated macrophages) are activated by Th2-related factors, express high level of anti-inflammatory cytokines, such as IL-10, and facilitate tumor progression (Biswas and Mantovani; Nat Immunol. 2010 October; 11(10):889-96). The pro-tumoral effects of M2 include the promotion of angiogenesis, advancement of invasion and metastasis, and the protection of the tumor cells from chemotherapy-induced apoptosis (Hu et al., Tumour Biol. 2015 December; 36(12): 9119-9126, and references therein). Tumor-associated macrophages are thought be of M2-like phenotype and have a protumor role. Galectin-9 has been shown to mediate myeloid cell differentiation toward an M2 phenotype (Enninga et al., Melanoma Res. 2016 October; 26(5):429-41). It is possible that Galectin-9 binding CD206 may result in reprogramming TAMs towards the M2 phenotype, similar to what has been previously shown for Dectin. Without wishing to be bound by theory, blocking the interaction of Galectin-9 with CD206 may provide one mechanism by which an anti-Galectin-9 antibody, e.g., a G9.2-17 antibody, can be therapeutically beneficial. Accordingly, in some embodiments, the anti-Galectin-9 antibodies described herein disrupt the interaction between Galectin-9 and CD206.

Galectin-9 has also been shown to interact with protein disulfide isomerase (PDI) and 4-1BB (Bi S, et al. Proc Natl Acad Sci USA. 2011; 108(26):10650-5; Madireddi et al. J Exp Med. 2014; 211(7):1433-48).

Anti-Galectin-9 antibodies can serve as therapeutic agents for treating diseases associated with Galectin-9 (e.g., those in which a Galectin-9 signaling plays a role). Without being bound by theory, an anti-Galectin-9 antibody may block a signaling pathway mediated by Galectin-9. For example, the antibody may interfere with the interaction between Galectin-9 and its binding partner (e.g., Dectin-1, TIM-3 or CD206), thereby blocking the signaling triggered by the Galectin-9/Ligand interaction. Alternatively, or in addition, an anti-Galectin-9 antibody may also exert its therapeutic effect by inducing blockade and/or cytotoxicity, for example, ADCC, CDC, or ADCP against pathologic cells that express Galectin-9. A pathologic cell refers to a cell that contributes to the initiation and/or development of a disease, either directly or indirectly.

The anti-Galectin-9 antibodies disclosed herein are capable of suppressing the signaling mediated by Galectin-9 (e.g., the signaling pathway mediated by Galectin-9/Dectin-1 or Galectin-9/Tim-3) or eliminating pathologic cells expressing Galectin-9 via, e.g., ADCC. Accordingly, the anti-Galectin-9 antibodies described herein can be used for inhibiting any of the Galectin-9 signaling and/or eliminating Galectin-9 positive pathologic cells, thereby benefiting treatment of diseases associated with Galectin-9.

Anti-Galectin-9 antibodies such as G9.2-17 were found to be effective in inducing apoptosis against cells expressing Galectin-9. Further, the anti-tumor effects of anti-Galectin-9 antibodies such as G9.2-17 were demonstrated in a mouse model, either by itself, or in combination with a checkpoint inhibitor (e.g., an anti-PD-1 antibody). As reported herein, the efficacy of G9.2-17 was tested in mouse models of PDAC and melanoma as well as in patient derived organoid tumor models (PDOTs). The orthotopic PDAC KPC mouse model (LSL-KrasG12D/+; LSL-Trp53R172H/+; Pdx-1-Cre) that was used recapitulates many features of human disease, including unresponsiveness to approved checkpoint inhibitors (Bisht and Feldmann G; Animal models for modeling pancreatic cancer and novel drug discovery; Expert Opin Drug Discov. 2019; 14(2):127-142; Weidenhofer et al., Animal models of pancreatic cancer and their application in clinical research; Gastrointestinal Cancer: Targets and Therapy 2016; 6). The B16F10 melanoma mouse model has been a long standing standard to test immunotherapies (Curran et al., PD-1 and CTLA-4 combination blockade expands infiltrating T cells and reduces regulatory T and myeloid cells within B16 melanoma tumors; Proc Natl Acad Sci USA. 2010; 107(9):4275-4280).

PDOTs isolated from fresh human tumor samples retain autologous lymphoid and myeloid cell populations, including antigen-experienced tumor infiltrating CD4 and CD8 T lymphocytes, and respond to immune therapies in short-term ex vivo culture (Jenkins et al. Ex Vivo Profiling of PD-1 Blockade Using Organotypic Tumor Spheroids. Cancer Discov. 2018; 8(2):196-215; Aref et al., 3D microfluidic ex vivo culture of organotypic tumor spheroids to model immune checkpoint blockade; Lab Chip. 2018; 18(20):3129-3143). As reported herein, expression of Galectin-9 on cancer cells was observed in patient-derived organoid assays.

In vivo studies were performed with G9.2-17 mouse IgG1 (G9.2-17 mIgG1 contains the exact same binding epitope as G9.2-17 human IgG4 and has the same effector function), which achieves significant reduction of tumor growth already as a single agent in the orthotopic KPC model, where approved checkpoint inhibitors do not work. In the B16F10 model G9.2-17 significantly exceeds the efficacy of anti-PD1. In both models, modulation of the intra-tumoral immune microenvironment using G9.2-17 mIgG1 through the upregulation of effector T cell activity and inhibition of immunosuppressive signals, as well as the augmentation of intra-tumoral CD8 T cell infiltration was demonstrated.

These results demonstrate that the anti-tumor methods disclosed herein, involving an anti-Galectin-9 antibody, optionally in combination the checkpoint inhibitor, would achieve superior therapeutic efficacy against the target solid tumors.

Accordingly, described herein are therapeutic uses of anti-Galectin-9 antibodies for treating certain cancers as disclosed herein.

### Antibodies Binding to Galectin-9

The present disclosure provides anti-Galectin-9 antibody G9.2-17 and functional variants thereof for use in the treatment methods disclosed herein.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the IMGT” numbering scheme, the “AHo” numbering scheme, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004); MacCallum et al., J. Mol. Biol. 262:732-745 (1996), Lefranc M P et al., Dev Comp Immunol, 2003 January; 27(1):55-77; and Honegger A and Pluckthun A, J Mol Biol, Jun. 8, 2001; 309(3):657-70. See also hgmp.mrc.ac.uk and bioinf.org.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)2 fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

Reference antibody G9.2-17 refers to an antibody capable of binding to human Galectin-9 and comprises a heavy chain variable region of SEQ ID NO: 7 and a light chain variable domain of SEQ ID NO: 8, both of which are provided below. In some embodiments, the anti-Galectin-9 antibody for use in the methods disclosed herein is the G9.2-17 antibody. In some embodiments, the anti-Galectin-9 antibody for use in the methods disclosed herein is an antibody having the same heavy chain complementary determining regions (CDRs) as reference antibody G9.2-17 and/or the same light chain complementary determining regions as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinforg.uk/abs/).

The heavy and light chain CDRs of reference antibody G9.2-17 is provided in Table 1 below (determined using the Kabat methodology):

TABLE 1
Heavy and Light Chain CDRs of G9.2-17

| | | | |
|---|---|---|---|
| G9.2-17 | V_{L} CDR1 | RASQSVSSAVA | SEQ ID NO: 1 |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYTYYADSVKG | SEQ ID NO: 5 |
| | V_{H} CDR3 | YWSYPSWWPYRGMDY | SEQ ID NO: 6 |

In some examples, the anti-Galectin-9 antibody for use in the methods disclosed herein may comprise (following the Kabat scheme) a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6 and/or may comprise a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3. The anti-Galectin-9 antibody, including the reference antibody G9.2-17, can be in any format as disclosed herein, for example, a full-length antibody or a Fab. The term “G9.2-17 (Ig4)” used herein refers to a G9.2-17 antibody which is an IgG4 molecule. Likewise, the term “G9.2-17 (Fab)” refers to a G9.2-17 antibody, which is a Fab molecule.

In some embodiments, the anti-Galectin-9 antibody or binding portion thereof comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody or binding portion thereof comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

Additional Galectin-9 antibodies, e.g., which bind to the CRD1 and/or CRD2 region of Galectin-9 are described in co-owned, co-pending U.S. patent application Ser. No. 16/173,970 and in co-owned, co-pending International Patent Applications PCT/US18/58028 and PCT/US2020/024767, the contents of each of which are herein incorporated by reference in their entireties.

In some embodiments, the anti-Galectin-9 antibody disclosed herein comprises light chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{L} CDRs of reference antibody G9.2-17. Alternatively or in addition, in some embodiments, the anti-Galectin-9 antibody comprises heavy chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{H} CDRs of reference antibody G9.2-17.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variations(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{H} that comprises the LC CDR1, LC CDR2, and LC CDR3, which collectively contain up to 8 amino acid residue variations (8, 7, 6, 5, 4, 3, 2, or 1 variations(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-Galectin-9 antibodies disclosed herein, having the heavy chain CDRs disclosed herein, contains framework regions derived from a subclass of germline V_{H} fragment. Such germline V_{H} regions are well known in the art. See, e.g., the IMGT database (www.imgt.org) or at www.vbase2.org/vbstat.php. Examples include the IGHV1 subfamily (e.g., IGHV1-2, IGHV1-3, IGHV1-8, IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9, IGHV3-11, IGHV3-13, IGHV3-15, IGHV3-20, IGHV3-21, IGHV3-23, IGHV3-30, IGHV3-33, IGHV3-43, IGHV3-48, IGHV3-49, IGHV3-53, IGHV3-64, IGHV3-66, IGHV3-72, and IGHV3-73, IGHV3-74), the IGHV4 subfamily (e.g., IGHV4-4, IGHV4-28, IGHV4-31, IGHV4-34, IGHV4-39, IGHV4-59, IGHV4-61, and IGHV4-B), the IGHV subfamily (e.g., IGHV5-51, or IGHV6-1), and the IGHV7 subfamily (e.g., IGHV7-4-1).

Alternatively or in addition, in some embodiments, the anti-Galectin-9 antibody, having the light chain CDRs disclosed herein, contains framework regions derived from a germline V_{κ} fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20), and an IGKV4 framework (e.g., IGKV4-1). In other instances, the anti-Galectin-9 antibody comprises a light chain variable region that contains a framework derived from a germline Vλ fragment. Examples include an IGλ1 framework (e.g., IGλV1-36, IGλV1-40, IGλV1-44, IGλV1-47, IGλV1-51), an IGλ2 framework (e.g., IGλV2-8, IGλV2-11, IGλV2-14, IGλV2-18, IGλV2-23), an IGλ3 framework (e.g., IGλV3-1, IGλV3-10, IGλV3-12, IGλV3-16, IGλV3-19, 21, IGλV3-25, IGλV3-27), an IGλ4 framework (e.g., IGλV4-3, IGλV4-60, IGλV4-69), an IGλ5 framework (e.g., IGλV5-39, IGλV5-45), an IGλ6 framework (e.g., IGλV6-57), an IGλ7 framework (e.g., IGλV7-43, IGλV7-46), an IGλ8 framework (e.g., IGλV8-61), an IGλ9 framework (e.g., IGλV9-49), or an IGλ10 framework (e.g., IGλV10-54).

In some embodiments, the anti-Galectin-9 antibody for use in the method disclosed herein can be an antibody having the same heavy chain variable region (V_{H}) and/or the same light chain variable region (V_{L}) as reference antibody G9.2-17, the V_{H} and V_{L} region amino acid sequences are provided below:

V_{H}:
(SEQ ID NO: 7)
EVQLVESGGGLVQPGGSLRLSCAASGFTVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSS
V_{L}:
(SEQ ID NO: 8)
DIQMTQSPSSLSASVGDRVTITCRASQSVSSAVAWYQQKPGKAPKLLIYS
ASSLYSGVPSRFSGSRSGTDFTLTISSLQPEDFATYYCQQSSTDPITFGQ
GTKVEIKR

In some embodiments, the anti-Galectin-9 antibody has at least 80% sequence identity (e.g., 80%, 85%, 90%, 91%, 92%, 93%, 94%, 95%, 96%, 97%, 98%, or 99% identity) to the heavy chain variable region of SEQ ID NO: 7. Alternatively or in addition, the anti-Galectin-9 antibody has at least 80% sequence identity (e.g., 80%, 85%, 90%, 91%, 92%, 93%, 94%, 95%, 96%, 97%, 98%, or 99% identity) to the light chain variable region of SEQ ID NO: 8.

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the VH and/or VL of G9.2-17 as disclosed herein) with substantially similar binding affinity (e.g., having a KD value in the same order) to human Galectin-9.

In some embodiments, the anti-Galectin-9 antibody as described herein can bind and inhibit the activity of Galectin-9 by at least 20% (e.g., 31%, 35%, 40%, 45%, 50%, 60%, 70%, 80%, 90%, 95% or greater, including any increment therein). The apparent inhibition constant (Ki^{app} or K_{i,app}), which provides a measure of inhibitor potency, is related to the concentration of inhibitor required to reduce enzyme activity and is not dependent on enzyme concentrations. The inhibitory activity of an anti-Galectin-9 antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{\begin{matrix}( [ E ] - [ I ] - {K}_{i}^{app} ) + \\ \sqrt{{( [ E ] - [ I ] - {K}_{i}^{app} )}^{2} + 4 [ E ] \cdot } {K}_{i}^{app}\end{matrix}}{2} \qquad ( Equation 1 )$$

Where A is equivalent to νₒ/E, the initial velocity (νₒ) of the enzymatic reaction in the absence of inhibitor (I) divided by the total enzyme concentration (E). In some embodiments, the anti-Galectin-9 antibody described herein has a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope. In some embodiments, the anti-Galectin-9 antibody has a lower Ki^{app} for a first target (e.g., the CRD2 of Galectin-9) relative to a second target (e.g., CRD1 of the Galectin-9). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the anti-Galectin-9 antibody inhibits a first antigen (e.g., a first protein in a first conformation or mimic thereof) greater relative to a second antigen (e.g., the same first protein in a second conformation or mimic thereof; or a second protein). In some embodiments, any of the anti-Galectin-9 antibodies is further affinity matured to reduce the Ki^{app} of the antibody to the target antigen or antigenic epitope thereof.

In some embodiments, the anti-Galectin-9 antibody suppresses Dectin-1 signaling, e.g., in tumor infiltrating immune cells, such as macrophages. In some embodiments, the anti-Galectin-9 antibody suppresses Dectin-1 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. Alternatively or in addition, the anti-Galectin-9 antibody suppresses the T cell immunoglobulin mucin-3 (TIM-3) signaling initiated by Galectin-9. In some embodiments, the anti-Galectin-9 antibody suppresses the T cell immunoglobulin mucin-3 (TIM-3) signaling, e.g., in tumor infiltrating immune cells, e.g., in some embodiments, by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody suppresses the CD206 signaling, e.g., in tumor infiltrating immune cells. In some embodiments, the anti-Galectin-9 antibody suppresses the CD206 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, the anti-Galectin-9 antibody blocks or prevents binding of Galectin-9 to CD206 by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody induces cell cytotoxicity, such as ADCC, in target cells expressing Galectin-9, e.g., wherein the target cells are cancer cells or immune suppressive immune cells. In some embodiments, the anti-Galectin-9 antibody induces apoptosis in immune cells, such as T cells, or cancer cells by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, any of the anti-Galectin-9 antibodies described herein induce cell cytotoxicity such as complement-dependent cytotoxicity (CDC) against target cells expressing Galectin-9.

Antibody-dependent cell-mediated phagocytosis (ADCP) is an important mechanism of action for antibodies that mediate part or all of their action though phagocytosis. In that case, antibodies mediate uptake of specific antigens by antigen presenting cells. ADCP can be mediated by monocytes, macrophages, neutrophils, and dendritic cells, through FcγRIIa, FcγRT, and FcγRIIIa, of which FcγRIIa (CD32a) on macrophages represent the predominant pathway.

In some embodiments, the anti-Galectin-9 antibody induces cell phagocytosis of target cells, e.g., cancer cells or immune suppressive immune cells expressing Galectin-9 (ADCP). In some embodiments, the anti-Galectin-9 antibody increases phagocytosis of target cells, e.g., cancer cells or immune suppressive immune cells, by at least 30% (e.g., 31%, 35%, 40%, 50%, 60%, 70%, 80%, 90%, 95% or greater, including any increment therein).

In some embodiments, the anti-Galectin-9 antibody described herein induces cell cytotoxicity such as complement-dependent cytotoxicity (CDC) against target cells, e.g., cancer cells or immune suppressive immune cells. In some embodiments, the anti-Galectin-9 antibody increases CDC against target cells by at least 30% (e.g., 31%, 35%, 40%, 50%, 60%, 70%, 80%, 90%, 95% or greater, including any increment therein).

In some embodiments, the anti-Galectin-9 antibody induces T cell activation, e.g., in tumor infiltrating T cells, i.e., suppress Galectin-9 mediated inhibition of T cell activation, either directly or indirectly. In some embodiments, the anti-Galectin-9 antibody promotes T cell activation by at least 30% (e.g., 31%, 35%, 40%, 50%, 60%, 70%, 80%, 90%, 95% or greater, including any increment therein). T cell activation can be determined by conventional methods, such as using well-known assays for measuring cytokines and checkpoint inhibitors (e.g., measurement of CD44, TNF alpha, IFNgamma, and/or PD-1). In some embodiments, the anti-Galectin-9 antibody promotes CD4+ cell activation by at least 30% (e.g., 31%, 35%, 40%, 50%, 60%, 70%, 80%, 90%, 95% or greater, including any increment therein). In a non-limiting example, the anti-Galectin antibody induces CD44 expression in CD4+ cells. In some embodiments, the anti-Galectin-9 antibody increases CD44 expression in CD4+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD4+ cells. In some embodiments, the anti-Galectin-9 antibody increases IFNgamma expression in CD4+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD4+ cells. In some embodiments, the anti-Galectin-9 antibody increases TNFalpha expression in CD4+ cells by at least 30% (e.g., 31%, 35%, 40%, 50%, 60%, 70%, 80%, 90%, 95% or greater, including any increment therein).

In some embodiments, the anti-Galectin-9 antibody promotes CD8+ cell activation by at least 30% (e.g., 31%, 35%, 40%, 50%, 60%, 70%, 80%, 90%, 95% or greater), including any increment therein). In a non-limiting example, the anti-Galectin antibody induces CD44 expression in CD8+ cells. In some embodiments, the anti-Galectin-9 antibody increases CD44 expression in CD8+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD8+ cells. In some embodiments, the anti-Galectin-9 antibody increases IFNgamma expression in CD8+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD8+ cells. In some embodiments, the anti-Galectin-9 antibody increases TNFalpha expression in CD8+ cells by at least 30% (e.g., 31%, 35%, 40%, 50%, 60%, 70%, 80%, 90%, 95% or greater, including any increment therein).

In some embodiments, an anti-Galectin-9 antibody as described herein has a suitable binding affinity for the target antigen (e.g., Galectin-9) or antigenic epitopes thereof. As used herein, “binding affinity” refers to the apparent association constant or K_{A}. The K_{A} is the reciprocal of the dissociation constant (K_{D}). The anti-Galectin-9 antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20).

These techniques can be used to measure the concentration of bound binding protein as a function of target protein concentration. Under certain conditions, the fractional concentration of bound binding protein ([Bound]/[Total]) is generally related to the concentration of total target protein ([Target]) by the following equation:

[Bound]/[Total]=[Target]/(Kd+[Target])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases tight binding is beneficial, but in other cases tight binding is not as desirable in vivo, and an antibody with lower binding affinity is more desirable.

In some embodiments, the heavy chain of any of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprise a single domain (e.g., CH1, CH2, or CH3) or a combination of any of the single domains, of a constant region (e.g., SEQ ID NO: 4, 5, 6). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement C1q binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK*
hIgG1 LALA Heavy Chain Constant Region
(SEQ ID NO: 12)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKFNNYVDGVEVHNAKTKPREEQYNSTYRVVSVLTVLHQDWLNGKEYKC
KVSNKALGAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK*
hIgG4 Heavy Chain Constant Region
(SEQ ID NO: 13)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSPGK*
hIgG4 Heavy Chain Constant Region
(SEQ ID NO: 20)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSPGK*
hIgG4 mut Heavy Chain Constant Region
(SEQ ID NO: 21)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK*

In some embodiments, anti-Galectin-9 antibodies having any of the above heavy chain constant regions are paired with a light chain having the following light chain constant region:

Light Chain Constant Region
(SEQ ID NO: 11)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

Exemplary full length anti-Galectin-9 antibodies are provided below:

G9.2-17 hIgG1 Heavy Chain
(SEQ ID NO: 16)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLICLVKGFYPSDIAVEWESNGQPENNYKTTPPVLDSD
GSFFLYSKLTVDKSRWQQGNVESCSVMHEALHNHYTQKSLSLSPGK*
G9.2-17 hIgG1 LALA Heavy Chain
(SEQ ID NO: 17)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALGAPIE
KTISKAKGQPREPQVYTLPPSREEMTKNQVSLICLVKGFYPSDIAVEWESNGQPENNYKTTPPVLDSD
GSFFLYSKLIVDKSRWQQGNVESCSVMHEALHNHYTQKSLSLSPGK*
G9.2-17 hIgG4 Heavy Chain
(SEQ ID NO: 18)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLIVDKSRWQEGNVESCSVMHEALHNHYTQKSLSLSPGK*
G9.2-17 hIgG4 Heavy Chain
(SEQ ID NO: 22)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVESCSVMHEALHNHYTQKSLSLSLGK*
G9.2-17 hIgG4 Fab Arm Exchange mut Heavy Chain
(SEQ ID NO: 19)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVESCSVMHEALHNHYTQKSLSLSPGK*
G9.2-17 hIgG4 Fab Arm Exchange mut Heavy Chain
(SEQ ID NO: 23)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK*

Any of the above heavy chain can be paired with a Light Chain of (SEQ ID NO: 15) shown below:

DIQMTQSPSSLSASVGDRVTITCRASQSVSSAVAWYQQKPGKAPKLLIYS
ASSLYSGVPSRFSGSRSGTDFTLTISSLQPEDFATYYCQQSSTDPITFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC*

In some embodiments, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 10. In one embodiment, the constant region of the anti-Galectin-9 antibody comprises a heavy chain IgG4 constant region comprising SEQ ID NO: 10. In one embodiment, the constant region of the anti-Galectin-9 antibody comprises a heavy chain IgG1 constant region consisting of SEQ ID NO: 10.

In some embodiments, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the constant region of the anti-Galectin-9 antibody comprises a heavy chain IgG4 constant region comprising SEQ ID NO: 20. In one embodiment, the constant region of the anti-Galectin-9 antibody comprises a heavy chain IgG4 constant region consisting of SEQ ID NO: 20.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 13. In one embodiment, the anti-Galectin-9 antibody comprises a heavy chain IgG4 constant region comprising SEQ ID NO: 13. In one embodiment, the anti-Galectin-9 antibody comprises a heavy chain IgG4 constant region consisting of SEQ ID NO: 13.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the anti-Galectin-9 antibody comprises a heavy chain IgG4 constant region comprising SEQ ID NO: 20. In one embodiment, the anti-Galectin-9 antibody comprises a heavy chain IgG4 constant region consisting of SEQ ID NO: 20.

In any of these embodiments, the anti-Galectin-9 antibody comprises a light chain constant region that has at least 80% (e.g., 80%, 85%, 90%, 91%, 92%, 93%, 94%, 95%, 96%, 97%, 98%, or 99% and any increment therein) sequence identity to SEQ ID NO: 11. In some embodiments, the anti-Galectin-9 antibody comprises a light chain constant region comprising SEQ ID NO: 11. In some embodiments, the anti-Galectin-9 antibody comprises a light chain constant region consisting of SEQ ID NO: 11.

In some embodiments, the IgG is a mutant with minimal Fc receptor engagement. In one example, the constant region is from a human IgG1 LALA. In one embodiment, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 12. In one embodiment, the anti-Galectin-9 antibody comprises a heavy chain IgG1 constant region comprising SEQ ID NO: 12. In one embodiment, the anti-Galectin-9 antibody comprises a heavy chain IgG1 constant region consisting of SEQ ID NO: 12.

In some embodiments, the anti-Galectin-9 antibody comprises a modified constant region. In some embodiments, the anti-Galectin-9 antibody comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8. In some embodiments, the IgG4 constant region is a mutant with reduced heavy chain exchange. In some embodiments, the constant region is from a human IgG4 Fab Arm Exchange mutant S228P.

In one embodiment, the constant region of the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 14. In one embodiment, the constant region of the anti-Galectin-9 antibody comprises a heavy chain IgG4 constant region comprising SEQ ID NO: 14. In one embodiment, the constant region of the anti-Galectin-9 antibody comprises a heavy chain IgG4 constant region consisting of SEQ ID NO: 14.

In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 21. In one embodiment, the anti-Galectin-9 antibody comprises a heavy chain IgG4 constant region comprising SEQ ID NO: 21. In one embodiment, the anti-Galectin-9 antibody comprises a heavy chain IgG4 constant region consisting of SEQ ID NO: 21.

In some embodiments, the anti-Galectin-9 antibody has chains corresponding to SEQ ID NO: 15 for the light chains; and the amino acid sequences of exemplary heavy chains correspond to SEQ ID NO: 10 (hIgG1); 12 (hIgG1 LALA); 13 (hIgG4); 20 (hIgG4); 14 (hIgG4 mut); and 21 (hIgG4 mut).

In some embodiments, the anti-Galectin-9 antibody has a light chain comprising, consisting essentially of, or consisting of SEQ ID NO: 15. In some embodiments, the anti-Galectin-9 antibody has a heavy chain comprising, consisting essentially of, or consisting of any one of the sequences selected from the group consisting of SEQ ID NO: 16-19, 22 and 23. In some embodiments, the anti-Galectin-9 antibody has a light chain comprising, consisting essentially of, or consisting of SEQ ID NO: 15 and a heavy chain comprising, consisting essentially of, or consisting of any one of the sequences selected from the group consisting of SEQ ID NO: 16-19. In some embodiments, the anti-Galectin-9 antibody has a light chain comprising SEQ ID NO: 15 and a heavy chain comprising any one of the sequences selected from the group consisting of SEQ ID NO: 16-19, 22 and 23. In some embodiments, the anti-Galectin-9 antibody has a light chain consisting essentially of SEQ ID NO: 15 and a heavy chain consisting essentially of any one of the sequences selected from the group consisting of SEQ ID NO: 16-19, 22 and 23. In some embodiments, the anti-Galectin-9 antibody has a light chain consisting of SEQ ID NO: 15 and a heavy chain consisting of any one of the sequences selected from the group consisting of SEQ ID NO: 16-19, 22 and 23. In one specific embodiment, the anti-Galectin-9 antibody has a light chain consisting essentially of SEQ ID NO: 15 and a heavy chain consisting essentially of SEQ ID NO: 19. In another specific embodiment, the anti-Galectin-9 antibody has a light chain consisting essentially of SEQ ID NO: 15 and a heavy chain consisting essentially of SEQ ID NO: 20.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 16. In one embodiment, the anti-Galectin-9 antibody comprises a heavy chain sequence comprising SEQ ID NO: 16. In one embodiment, the anti-Galectin-9 antibody comprises a heavy chain sequence consisting of SEQ ID NO: 16.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 17. In one embodiment, the anti-Galectin-9 antibody comprises a heavy chain sequence comprising SEQ ID NO: 17. In one embodiment, the anti-Galectin-9 antibody comprises a heavy chain sequence consisting of SEQ ID NO: 17.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 18. In one embodiment, the anti-Galectin-9 antibody comprises a heavy chain sequence comprising SEQ ID NO: 18. In one embodiment, the anti-Galectin-9 antibody comprises a heavy chain sequence consisting of SEQ ID NO: 18.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 22. In one embodiment, the anti-Galectin-9 antibody comprises a heavy chain sequence comprising SEQ ID NO: 22. In one embodiment, the anti-Galectin-9 antibody comprises a heavy chain sequence consisting of SEQ ID NO: 22.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 19. In one embodiment, the anti-Galectin-9 antibody comprises a heavy chain sequence comprising SEQ ID NO: 19. In one embodiment, the anti-Galectin-9 antibody comprises a heavy chain sequence consisting of SEQ ID NO: 19.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 23. In one embodiment, the anti-Galectin-9 antibody comprises a heavy chain sequence comprising SEQ ID NO: 23. In one embodiment, the anti-Galectin-9 antibody comprises a heavy chain sequence consisting of SEQ ID NO: 23.

In any of these embodiments, the anti-Galectin-9 antibody comprises a light chain sequence having at least 80% (e.g., 80%, 85%, 90%, 91%, 92%, 93%, 94%, 95%, 96%, 97%, 98%, or 99% and any increment therein) sequence identity to SEQ ID NO: 15. In some embodiments, the anti-Galectin-9 antibody comprises a light chain sequence comprising SEQ ID NO: 15. In some embodiments, the anti-Galectin-9 antibody comprises a light chain sequence consisting of SEQ ID NO: 15.

In specific examples, the anti-Galectin-9 antibody used in the treatment methods disclosed herein has a heavy chain of SEQ ID NO:19 and a light chain of SEQ ID NO:15. In some embodiments, the anti-Galectin-9 antibody used in the treatment methods disclosed herein is G9.2-17 IgG4.

### Preparation of Anti-Galectin-9 Antibodies

Antibodies capable of binding Galectin-9 as described herein can be made by any method known in the art, including but not limited to, recombinant technology. One example is provided below.

Nucleic acids encoding the heavy and light chain of an anti-Galectin-9 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Galectin-9 antibody, as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-Galectin-9 antibody and the other encoding the light chain of the anti-Galectin-9 antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Galectin-9 antibody as described herein, vectors (e.g., expression vectors) containing such; and host cells comprising the vectors are within the scope of the present disclosure.

Anti-Galectin-9 antibodies thus prepared can be can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Dectin-1 and Galectin-9, and monitoring any one or more of the following characteristics: (a) binding between Dectin-1 and Galectin-9 and inhibition of the signaling transduction mediated by the binding; (b) preventing, ameliorating, or treating any aspect of a solid tumor; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping.

Additional assays to determine bioactivity of an anti-Galectin-9 antibody include measurement of CD8+ and CD4+ (conventional) T-cell activation (in an in vitro or in vivo assay, e.g., by measuring inflammatory cytokine levels, e.g., IFNgamma, TNFalpha, CD44, ICOS granzymeB, Perforin, IL2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

### Pharmaceutical Compositions

The anti-Galectin-9 antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Areiams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Areiams and Wilkins, Ed. K. E. Hoover). Acceptable carriers, excipients, or stabilizers are nontoxic to recipients at the dosages and concentrations used, and comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, the anti-Galectin-9 antibodies, or the encoding nucleic acid(s), are be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent are conveniently comprise between 0.05 and 5% surface-active agent, and can be between 0.1 and 2.5%. It are be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It are be appreciated that other ingredients may be added, for example glycerol or glucose, to adjust the tonicity of the emulsion. Suitable emulsions are typically contain up to 20% oil, for example, between 5 and 20%. The fat emulsion can comprise fat droplets between 0.1 and 1.0.im, particularly 0.1 and 0.5.im, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Methods of Treatment

The present disclosure provides methods for treating solid tumors such as PDA, CRC, HCC, and cholangiocarcinoma, using any of the anti-Galectin antibodies, for example G9.2-17, e.g., G9.2-17 IgG4, either alone or in combination with a checkpoint inhibitor such as an anti-PD-1 antibody. Any of the anti-Galectin-9 antibodies described herein can be used in any of the methods described herein. In some embodiments, the anti-Galectin-9 antibody is G9.2-17. Such antibodies can be used for treating diseases associated with Galectin-9. In some aspects, the invention provides methods of treating cancer. In some embodiments, the present disclosure methods for reducing, ameliorating, or eliminating one or more symptom(s) associated with cancer.

In some embodiments, the disclosure provides a method for treating a solid tumor in a subject, the method comprising administering to a subject in need thereof effective amount of an anti-Galectin-9 antibody or an effective amount of a pharmaceutical composition comprising an anti-Galectin-9 antibody described herein or antigen binding fragment thereof. In some embodiments, the anti-Galectin-9 antibody is an antibody having the same heavy chain CDR sequences and/or the same light chain CDR sequences as reference antibody G9.2-17. In some embodiments, the anti-Galectin-9 antibody is an antibody having the same VH and VL sequences as reference antibody G9.2-17. In some embodiments, such an antibody is an IgG1 molecule (e.g., having a wild-type IgG1 constant region or a mutant thereof as those disclosed herein). Alternatively, the antibody is an IgG4 molecule (e.g., having a wild-type IgG4 constant region or a mutant thereof as those described herein). In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain variable region comprising SEQ ID NO: 7 and a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In specific examples, the anti-Galectin-9 antibody used herein has a heavy chain of SEQ ID NO:19 and a light chain of SEQ ID NO:15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

Also within the scope of the present disclosure are pharmaceutical compositions for use in treating a solid tumor (e.g., those described herein and including metastatic solid tumors), and uses of any of the anti-Galectin-9 antibodies for manufacturing a medicament for treating the solid tumor, wherein the uses disclosed herein, in some embodiments, involve one or more of the treatment conditions (e.g., dose, dosing regimen, administration route, etc.) as also disclosed herein. In some embodiments, the antibody for use for manufacturing a medicament for treating a solid tumor comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain variable region comprising SEQ ID NO: 7 and a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody comprises a heavy chain comprising SEQ ID NO: 19 and a light chain comprising SEQ ID NO: 15. In some embodiments, the antibody is G9.2-17 IgG4. In some embodiments, the anti-Galectin-9 antibody for use for manufacturing a medicament for treating a solid tumor is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody for use for manufacturing a medicament for treating a solid tumor is administered once every two weeks, e.g., via intravenous infusion. In some embodiments, the anti-Galectin-9 antibody is administered once every 2 weeks for one cycle, once every 2 weeks for two cycles, once every 2 weeks for 3 cycles, once every 2 weeks for 4 cycles, or once every 2 weeks for more than 4 cycles. In some embodiments, the anti-Galectin-9 antibody is administered once every 2 weeks for 4 cycles. In some embodiments, the duration of treatment is 12-24 months or longer. In some embodiments, the cycles extend for a duration of 3 months to 6 months, or 6 months to 12 months or 12 months to 24 months or longer. In some embodiments, the cycle length is modified, e.g., temporarily or permanently to a longer duration, e.g., 3 weeks or 4 weeks. In some embodiments, the use further comprises administering to the subject an immune checkpoint inhibitor, e.g., an anti-PD1 antibody, as described herein, e.g., administered according to a regimen described herein. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

Given that pro-tumor action of Galectin-9 is mediated through interaction with immune cells (e.g., interactions with lymphoid cells via TIM-3, CD44, and 41BB, and with macrophages via dectin-1 and CD206) and given that Galectin-9 is expressed in a large number of tumors, targeting Galectin-9, e.g., using a Galectin-9 binding antibody to inhibit interaction with its receptors provides a therapeutic approach that can be applied across a variety of different tumor types.

In some embodiments, the disclosure provides a method for treating a solid tumor in a subject, the method comprising administering to a subject in need thereof an effective amount of an anti-Galectin-9 antibody described herein, including but not limited to, G9.2-17 IgG4. In some examples, the method disclosed herein is applied to a human patient having pancreatic cancer, for example, ductal adenocarcinoma (PDA). In some instances, the PDA patient may have a metastatic cancer. In some examples, the method disclosed herein is applied to a human patient having colorectal cancer (CRC). In some embodiments, the colorectal cancer is metastatic. In some examples, the method disclosed herein is applied to a human patient having hepatocellular carcinoma melanoma. In some embodiments, the hepatocellular carcinoma is metastatic. In other examples, the method disclosed herein is applied to a human patient having cholangiocarcinoma. In some embodiments, the cholangiocarcinoma is metastatic.

Pancreatic ductal adenocarcinoma (PDA) is a devastating disease with few long-term survivors (Yadav et al., Gastroenterology, 2013, 144, 1252-1261). Inflammation is paramount in PDA progression as oncogenic mutations alone, in the absence of concomitant inflammation, are insufficient for tumorigenesis (Guerra et al., Cancer Cell, 2007, 11, 291-302). Innate and adaptive immunity cooperate to promote tumor progression in PDA. In particular, specific innate immune subsets within the tumor microenvironment (TME) are apt at educating adaptive immune effector cells towards a tumor-permissive phenotype. Antigen presenting cell (APC) populations, including M2-polarized tumor-associated macrophages (TAMs) and myeloid dendritic cells (DC), induce the generation of immune suppressive Th2 cells in favor of tumor-protective Th1 cells (Ochi et al., J of Exp Med., 2012, 209, 1671-1687; Zhu et al., Cancer Res., 2014, 74, 5057-5069). Similarly, it has been shown that myeloid derived suppressor cells (MDSC) negate anti-tumor CD8⁺ cytotoxic T-Lymphocyte (CTL) responses in PDA and promote metastatic progression (Connolly et al., J Leuk Biol., 2010, 87, 713-725; Pylayeva-Gupta et al., Cancer Cell, 2012, 21, 836-847; Bayne et al., Cancer Cell, 2012, 21, 822-835).

Pancreatic cancer remains a disease that is difficult to treat due to a typically late presentation, relatively high resistance to chemotherapy, and lack of effective immune and targeted therapies. Globally, approximately 455,000 new cases of pancreatic cancer have been reported in 2018, and an estimated 355,000 new cases are estimated to occur until 2040 annually, and almost as many deaths are reported as new cases on a yearly basis. It is projected to be the second leading cause of cancer-related deaths in the United States by the year 2030. Despite intervention, the median life expectancy for patients with metastatic pancreatic cancer is less than 1 year with current treatment, while most patients (as many as 80%) present at an advanced/metastatic stage, when the disease is beyond curative resection. Despite advancements in the detection and management of pancreatic cancer, the five-year survival rate of metastatic disease remains at ten percent. The current standard of care for metastatic pancreatic cancer is predominantly chemotherapy, while a distinct minority of patients (under ten percent) with BRCA1/2 mutations and mismatch repair deficient tumors may benefit from PARP inhibitors and potentially anti-PD-1 therapy. However, for the vast majority of patients with this disease, currently approved immunotherapies have been generally unsuccessful due to a highly immunosuppressive environment.

Colorectal cancer (CRC), also known as bowel cancer, colon cancer, or rectal cancer, is any cancer affecting the colon and the rectum. CRC is known to be driven by genetic alterations of tumor cells and is also influenced by tumor-host interactions. Recent reports have demonstrated a direct correlation between the densities of certain T lymphocyte subpopulations and a favorable clinical outcome in CRC, supporting a major role of T-cell-mediated immunity in repressing tumor progression of CRC.

Colorectal cancer presents one of the largest cancer burdens in the world, with approximately 700,000 people diagnosed globally each year. Despite significant advances in standard of care therapies, the five-year survival rate for metastatic colorectal cancer (CRC), remains around 12 percent. Death from CRC is expected to nearly double within the next 20 years. The current standard of care for CRC are chemotherapy regimens, combined and/or in sequence with anti-angiogenic therapy and anti-EGFR modalities. In addition, current immunotherapies are only efficacious (albeit producing profound and durable responses) in less than 20% of patients whose tumors demonstrate mismatch repair deficiency. Outcomes on immunotherapy in microsatellite stable CRC, which are the majority of patients with CRC are suboptimal and novel therapeutic strategies are needed.

Hepatocellular carcinoma (HCC) is the most common type of primary liver cancer. Hepatocellular carcinoma occurs most often in people with chronic liver diseases, such as cirrhosis caused by hepatitis B or hepatitis C infection. HCC is usually accompanied by cirrhotic liver with extensive lymphocyte infiltration due to chronic viral infection. Many studies have demonstrated that tumor-infiltrating effector CD8+ T cells and T helper 17 (Th17) cells correlate with improved survival after surgical resection of tumors. However, tumor-infiltrating effector T cells fail to control tumor growth and metastasis (Pang et al., Cancer Immunol Immunother 2009; 58:877-886).

Cholangiocarcinoma is a group of cancers that begin in the bile ducts. Cholangiocarcinoma is commonly classified by its location in relation to the liver. For example, intrahepatic cholangiocarcinoma, accounting for less than 10% of all cholangiocarcinoma cases, begins in the small bile ducts within the liver. In another example, perihilar cholangiocarcinoma (also known as a Klatskin tumor), accounting for more than half of the cholangiocarcinoma cases, begins in hilum, where two major bile ducts join and leave the liver. Others are classified as distal cholangiocarcinomas, which begin in bile ducts outside the liver.

Cholangiocarcinomas are aggressive tumors, and most patients have advanced-stage disease at presentation. The incidence of cholangiocarcinoma is rising, and effective therapies are urgently needed. Gemcitabine plus cisplatin remains the standard first-line systemic therapy for advanced cholangiocarcinoma, although it leaves much to be desired, as median survival is less than one year. Beyond failure of first line therapy, available evidence to guide therapeutic decisions is scarce. Triple chemotherapy (nab-paclitaxel plus gemcitabine-cisplatin) regimen may be approved in the future, as well as FGFR2 inhibitors in selected cohorts. However, suboptimal response rates to immunotherapy in human clinical trials imply that the preponderance of cholangiocarcinomas are immune ‘cold’ tumors with a non-T cell infiltrated microenvironment. In fact, immunotherapy to date has not produced response rates exceeding 17 percent and as of the date of the instant application, no immune oncology agents have been approved.

A subject having any of the above noted cancers can be identified by routine medical examination, e.g., laboratory tests, organ functional tests, genetic tests, interventional procedure (biopsy, surgery) any and all relevant imaging modalities. In some embodiments, the subject to be treated by the method described herein is a human cancer patient who has undergone or is subjecting to an anti-cancer therapy, for example, chemotherapy, radiotherapy, immunotherapy, or surgery. In some embodiments, subjects have received prior immune-modulatory anti-tumor agents. Non-limiting examples of such immune-modulatory agents include, but are not limited to as anti-PD1, anti-PD-L1, anti-CTLA-4, anti-OX40, anti-CD137, etc. In some embodiments, the subject shows disease progression through the treatment. In other embodiments, the subject is resistant to the treatment (either de novo or acquired). In some embodiments, such a subject is demonstrated as having advanced malignancies (e.g., inoperable or metastatic). Alternatively or in addition, in some embodiments, the subject has no standard therapeutic options available or ineligible for standard treatment options, which refer to therapies commonly used in clinical settings for treating the corresponding solid tumor.

In some instances, the subject may be a human patient having a refractory disease, for example, a refractory PDA, a refractory CRC, a refractory HCC, or a refractory cholangiocarcinoma. As used herein, “refractory” refers to the tumor that does not respond to or becomes resistant to a treatment. In some instances, the subject may be a human patient having a relapsed disease, for example, a relapsed PDA, a relapsed CRC, a relapsed HCC, or a relapsed cholangiocarcinoma. As used herein, “relapsed” or “relapses” refers to the tumor that returns or progresses following a period of improvement (e.g., a partial or complete response) with treatment.

In some embodiments, the human patient to be treated by the methods disclosed herein meets one or more of the inclusion and exclusion criteria disclosed in Example 1 below. For example, the human patient may be 18 or older; having histologically confirmed unresectable metastatic or inoperable cancer (e.g., without standard therapeutic options), having a life expectancy >3 months, having recent archival tumor sample available for biomarker analysis (e.g., an archival species for Galectin-9 tumor tissue expression levels assessed by IHC); having a measurable disease, according to RECIST v1.1, having Eastern Cooperative Oncology Group (ECOG) performance status 0-1 or Karnofsky score >70; having no available standard of care options, having MSI-H (Microsatellite instability high and MSS (Microsatellite Stable); received at least one line of systemic therapy in the advanced/metastatic setting; having adequate hematologic and end organ function (defined in Example 1 below); having completed treatment for brain metastases if any (see Example 1 below); having no evidence of active infection and no serious infection within the past month; having at least four (4) weeks s or 5 half lives (whichever is shorter) since the last dose of anti-cancer therapy before the first anti-Gal-9 antibody administration; having continued bisphosphonate treatment (zolendronic acid) or denosumab for bone metastases if applicable. CCR or CCA patients subject to the instant treatment may have at least one prior line of therapy in the metastatic setting is required. In some embodiments, CCR or CCA patients subject to the instant treatment have had at least one prior line of therapy in the metastatic setting.

Alternatively or in addition, the subject suitable for the treatment disclosed herein may not have one or more of the following: diagnosed with metastatic cancer of an unknown primary; any active uncontrolled bleeding, and any patients with a bleeding diathesis (e.g., active peptic ulcer disease); receiving any other investigational agents within 4 weeks or 5 half-lives of anti-galectin-9 antibody administration; receiving radiation therapy within 4 weeks of the first dose of the anti-Galectin-9 antibody, except for palliative radiotherapy to a limited field, such as for the treatment of bone pain or a focally painful tumor mass; having fungating tumor masses; for PDAC patients, having prior gemcitabine containing regimen less than 6 months from the begin of the treatment, patients having locally advanced PDAC; having active clinically serious infection >grade 2 NCI-CTCAE version 5.0; having symptomatic or active brain metastases; having ≥CTCAE grade 3 toxicity (see details and exceptions in Example 1); having history of second malignancy (see exceptions in Example 1); having evidence of severe or uncontrolled systemic diseases, congestive cardiac failure; having serious non-healing wound, active ulcer or untreated bone fracture; having uncontrolled pleural effusion, pericardial effusion, or ascites requiring recurrent drainage procedures; having spinal cord compression not definitively treated with surgery and/or radiation. Leptomeningeal disease, active or previously treated; having significant vascular disease; having active auto-immune disorder (see exceptions in Example 1); require systemic immunosuppressive treatment; having tumor-related pain (>grade 3) unresponsive to broad analgesic interventions (oral and/or patches); having uncontrolled hypercalcemia, despite use of bisphosphonates; having any history of an immune-related Grade 4 adverse event attributed to prior checkpoint inhibitor therapy (CIT); received an organ transplant(s); and/or on undergoing dialysis; for HCC patients and/or CCA patients, having any ablative therapy prior to the treatment; hepatic encephalopathy or severe liver adenoma; having Child-Pugh score ≥7; having metastatic hepatocellular carcinoma that progressed while receiving at least one previous line of systemic therapy; having refuse or not toleratedsorafenib; or having had standard therapy considered ineffective, intolerable, or inappropriate or for which no effective standard therapy is available.

In some instances, the subject is a human patient having an elevated level of Galectin-9 as relative to a control level. The level of Galectin-9 can be a plasma or serum level of Galectin-9 in the human patient. In other examples, the level of Galectin-9 can be the level of cell-surface Galectin-9, for example the level of Galectin-9 on cancer cells. In one example, the level of Galectin-9 can be the level of surface Galectin-9 expressed on cancer cells in patient-derived organotypic tumor spheroids (PDOT), which can be prepared by, e.g., the method disclosed in Examples below. A control level may refer to the level of Galectin-9 in a matched sample of a subject of the same species (e.g., human) who is free of the solid tumor. In some examples, the control level represents the level of Galectin-9 in healthy subjects.

To identify such a subject, a suitable biological sample can be obtained from a subject who is suspected of having the solid tumor and the biological sample can be analyzed to determine the level of Galectin-9 contained therein (e.g., free, cell-surface expressed, or total) using conventional methods, e.g., ELISA or FACS. In some embodiments, organoid cultures are prepared, e.g., as described herein, and used to assess Galectin-9 levels in a subject. Single cells derived from certain fractions obtained as part of the organoid preparation process are also suitable for assessment of Galectin-9 levels in a subject. In some instances, an assay for measuring the level of Galectin-9, either in free form or expressed on cell surface, involves the use of an antibody that specifically binds the Galectin-9 (e.g., specifically binds human Galectin-9). Any of the anti-Galectin-9 antibodies known in the art can be tested for suitability in any of the assays described above and then used in such assays in a routine manner. In some embodiments, an antibody described herein (e.g., a G9.2-17 antibody) can be used in such as assay. In some embodiments, an antibody described in U.S. Pat. No. 10,344,091 and WO2019/084553, the relevant disclosures of each of which are incorporated by reference for the purpose and subject matter referenced herein. In some examples, the anti-Galectin-9 antibody is a Fab molecule. Assay methods for determining Galectin-9 levels as disclosed herein are also within the scope of the present disclosure.

An effective amount of the pharmaceutical composition described herein can be administered to a subject (e.g., a human) in need of the treatment via a suitable route, systemically or locally. In some embodiments, the anti-Galectin-9 antibodies are administered by intravenous administration, e.g., as a bolus or by continuous infusion over a period of time, by intramuscular, intraperitoneal, intracerobrospinal, subcutaneous, intra-arterial, intra-articular, intrasynovial, intrathecal, intratumoral, oral, inhalation or topical routes. In one embodiment, the anti-Galectin-9 antibody is administered to the subject by intravenous infusion. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, or increased anti-tumor immune responses in the tumor microenvironment. Non-limiting examples of increased anti-tumor responses include increased activation levels of effector T cells, or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein are determined empirically in individuals who have been given one or more administration(s) of the antibody. Individuals are given incremental dosages of the antagonist. To assess efficacy of the antagonist, an indicator of the disease/disorder can be followed.

In some instances, the anti-Galectin-9 antibody as disclosed herein (e.g., G9.2-17) can be administered to a subject at a suitable dose, for example, about 1 to about 32 mg/kg. Examples include 1 mg/kg to 3 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges. In some embodiments, the Galectin-9 antibody is administered at 2 mg/kg. In some embodiments, the Galectin-9 antibody is administered at 4 mg/kg. In some embodiments, the Galectin-9 antibody is administered at 8 mg/kg. In some embodiments, the Galectin-9 antibody is administered at 12 mg/kg. In some embodiments, the Galectin-9 antibody is administered at 16 mg/kg. In some instances, multiple doses of the anti-Galectin-9 antibody can be administered to a subject at a suitable interval or cycle, for example, once every two to four weeks (e.g., every two, three, or four weeks). The treatment may last for a suitable period, for example, up to 3 months, up to 6 months, or up to 12 months or up to 24 months.

In specific embodiments, the interval or cycle is 2 weeks. In some embodiments, the regimen is once every 2 weeks for one cycle, once every 2 weeks for two cycles, once every 2 weeks for three cycles, once every 2 weeks for four cycles, or once every 2 weeks for more than four cycles. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

In specific embodiments, the interval or cycle is 3 weeks. In some embodiments, the regimen is once every 3 weeks for one cycle, once every 3 weeks for two cycles, once every 3 weeks for three cycles, once every 3 weeks for four cycles, or once every 3 weeks for more than four cycles. In some embodiments, the treatment is once every 3 weeks for 1 to 3 months, once every 3 weeks for 3 to 6 months, once every 3 weeks for 6 to 12 months, or once every 3 weeks for 12 to 24 months, or longer.

In specific embodiments, the interval or cycle is 4 or more weeks. In some embodiments, the regimen is once every 4 or more weeks for one cycle, once every 4 or more weeks for two cycles, once every 4 or more weeks for three cycles, once every 4 or more weeks for four cycles, or once every 4 or more weeks for more than four cycles. In some embodiments, the treatment is once every 4 or more weeks for 1 to 3 months, once every 4 or more weeks for 3 to 6 months, once every 4 or more weeks for 6 to 12 months, or once every 4 or more weeks for 12 to 24 months, or longer. In some embodiments, the treatment is a combination of treatment at various time, e.g., a combination or 2 weeks, 3 weeks, 4 or more 4 weeks. In some embodiments, the treatment interval is adjusted in accordance with the patient's response to treatment. In some embodiments, the dosage(s) is adjusted in accordance with the patient's response to treatment. In some embodiments, the dosages are altered between treatment intervals. In some embodiments, the treatment may be temporarily stopped.

In some examples, the anti-Galectin-9 antibody is administered to a human patient having a target solid tumor as disclosed herein (e.g., PDA, CRC, HCC, or cholangiocarcinoma) at a dose of about 3 mg/kg once every two weeks via intravenous infusion. In other examples, the anti-Galectin-9 antibody is administered to the human patient having the target solid tumor at a dose of about 15 mg/kg once every two weeks via intravenous infusion.

The term “about” or “approximately” means within an acceptable error range for the particular value as determined by one of ordinary skill in the art, which are depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In some embodiments, the methods of the present disclosure increase anti-tumor activity (e.g., reduce cell proliferation, tumor growth, tumor volume, and/or tumor burden or load or reduce the number of metastatic lesions over time) by at least about 10%, 20%, 25%, 30%, 40%, 50%, 60%, 70%, 75%, 80%, 85%, 90%, 95%, or more as compared to levels prior to treatment or in a control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more. In some embodiments, before, during, and after the administration of the pharmaceutical composition, cancerous cells and/or biomarkers in a subject are measured in a biological sample, such as blood, serum, plasma, urine, peritoneal fluid, and/or a biopsy from a tissue or organ. In some embodiments, the methods include administration of the compositions of the invention to reduce tumor volume, size, load or burden in a subject to an undetectable size, or to less than about 1%, 2%, 5%, 10%, 20%, 25%, 30%, 40%, 50%, 60%, 70%, 75%, 80%, or 90% of the subject's tumor volume, size, load or burden prior to treatment. In other embodiments, the methods include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment. In other embodiments, the methods include administration of the compositions of the invention to reduce the development of or the number or size of metastatic lesions in a subject to an undetectable rate, or to less than about 1%, 2%, 5%, 10%, 20%, 25%, 30%, 40%, 50%, 60%, 70%, 75%, 80%, or 90% of the rate prior to treatment.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

A response to treatment, e.g., a treatment of a solid tumor as described herein, can be assessed according to RECIST or the updated RECIST 1.1 criteria, as described in Example 1 below and Eisenhower et al., New response evaluation criteria in solid tumours: Revised RECIST guideline (version 1.1); European Journal Of Cancer 45 (2009) 228-247, the contents of which is herein incorporated by reference in its entirety.

In some embodiments, treating can improve the overall response (e.g., at 3, 6 or 12 months, or a later time), e.g., as compared to a baseline level prior to initiation of treatment or as compared to a control group not receiving the treatment. In some embodiments, treating can result in a complete response, a partial response or stable disease (e.g., as measured at 3 months, 6 months or 12 months, or at a later time). Such a response can be temporary over a certain time period or permanent. In some embodiments, treating can improve the likelihood of a complete response, a partial response or stable disease (e.g., as measured at 3 months, 6 months or 12 months, or at a later time), e.g., as compared to a control group not receiving the treatment. Such a response can be temporary over a certain time period or permanent. In some embodiments, treating can result in reduced or attenuated progressive disease (e.g., as measured at 3 months, 6 months or 12 months, or at a later time), e.g., as compared to a control group not receiving the treatment. Such an attenuation may be temporary or permanent.

A partial response is a decrease in the size of a tumor, or in the extent of cancer in the body, i.e., the tumor burden, in response to treatment as compared to a baseline level before the initiation of the treatment. For example, according to the RECIST response criteria, a partial response is defined as at least a 30% decrease in the sum of diameters of target lesions, taking as reference the baseline sum diameters. Progressive disease is a disease that is growing, spreading, or getting worse. For example, according to the RECIST response criteria, progressive disease includes disease in which at least a 20% increase in the sum of diameters of target lesions is observed, and the sum must also demonstrate an absolute increase of at least 5 mm. Additionally, the appearance of one or more new lesions is also considered progression. A tumor that is neither decreasing nor increasing in extent or severity as compared to a baseline level before initiation of the treatment is considered stable disease. For example, according to the RECIST response criteria, stable disease occurs when there is neither sufficient shrinkage to qualify for partial response nor sufficient increase to qualify for progressive disease, taking as reference the smallest sum diameters while on study.

Accordingly, in some embodiments, treating can result in overall tumor size reduction, maintenance of tumor size, either permanently or over a minimum time period, relative to a baseline tumor size prior to initiation of the treatment (e.g., as measured at 3 months, 6 months or 12 months, or at a later time). In some embodiments, treating can result in a greater likelihood of overall tumor size reduction or maintenance of tumor size, either permanent or over a minimum time period, e.g., as compared to a control group not receiving the treatment (e.g., as measured at 3 months, 6 months or 12 months, or at a later time). Tumor size, e.g., the diameters of tumors, can be measured according to methods known in the art, which include measurements from CT and MRI images in combination with various software tools, according to specific measurement protocols, e.g., as described in Eisenhower et al., referenced above. Accordingly, in some embodiments, tumor size is measured in regularly scheduled restaging scans (e.g., CT with contrast, MRI with contrast, PET-CT (diagnostic CT) and/or X-ray). In some embodiments, tumor size reduction, maintenance of tumor size refers to the size of target lesions. In some embodiments, tumor size reduction, maintenance of tumor size refers to the size of non-target lesions. According to RECIST 1.1, when more than one measurable lesion is present at baseline, all lesions up to a maximum of five lesions total (and a maximum of two lesions per organ) representative of all involved organs should be identified as target lesions. All other lesions (or sites of disease) including pathological lymph nodes should be identified as non-target lesions.

In some embodiments, treating can result in reduction of tumor burden, or maintenance of tumor burden as compared to baseline levels prior to initiation of the treatment (e.g., as measured at 3 months, 6 months or 12 months, or at a later time). The reduction in tumor burden can be temporary over a certain time period or permanent. In some embodiments, treating can result in in a greater likelihood of a reduction of tumor burden, or maintenance of tumor burden, e.g., as compared to a control group not receiving the treatment (e.g., as measured at 3 months, 6 months or 12 months, or at a later time). As used herein, tumor burden refers to amount of cancer, the size or the volume of the tumor in the body of a subject, accounting for all sites of disease. Tumor burden can be measured using methods known in the art, including but not limited to, FDG positron emission tomography (FDG-PET), magnetic resonance imaging (MRI), and optical imaging, comprising bioluminescence imaging (BLI) and fluorescence imaging (FLI).

In some embodiments, treating can result in an increase in the time to disease progression or in progression free survival (e.g., as measured at 3 months, 6 months or 12 months, or at a later time post initiation of treatment) as compared to a control group that does not receive the treatment. Progression free survival can be either permanent or progression free survival over a certain amount of time. In some embodiments, treating can result in a greater likelihood of progression free survival (either permanent progression free survival or progression free survival over a certain amount of time, e.g., 3, 6 or 12 months or e.g., as measured at 3 months, 6 months or 12 months, or at a later time post initiation of treatment) as compared to a control group that does not receive the treatment. Progression-free survival (PFS) is defined as the time from random assignment in a clinical trial, e.g., from initiation of a treatment to disease progression or death from any cause. In some embodiments, treating can result in longer survival or greater likelihood of survival, e.g., at a certain time, e.g., at 6 or 12 months.

A response to treatment, e.g., a treatment of a solid tumor as described herein, can be assessed according to iRECIST criteria, as described in Seymour et al, iRECIST: guidelines for response criteria for use in trials; The Lancet, Vol18, March 2017, the contents of which is herein incorporated by reference in its entirety. iRECIST was developed for the use of modified RECIST1.1 criteria specifically in cancer immunotherapy trials, to ensure consistent design and data collection and can be used as guidelines to a standard approach to solid tumor measurements and definitions for objective change in tumor size for use in trials in which an immunotherapy is used. iRECIST is based on RECIST 1.1. Responses assigned using iRECIST have a prefix of “i” (ie, immune)—e.g., “immune” complete response (iCR) or partial response (iPR), and unconfirmed progressive disease (iUPD) or confirmed progressive disease (iCPD) or stable disease (iSD) to differentiate them from responses assigned using RECIST 1.1, and all of which are defined in Seymour et al.

Accordingly, in some embodiments, treating can result in a “immune” complete response (iCR), a partial response (iPR) or stable disease (iSD) (e.g., as measured at 3 months, 6 months or 12 months, or at a later time), as compared to the baseline level of disease prior to initiation of the treatment. The reduction in the “immune” response, e.g., iCR, iPR, or iSD can be temporary over a certain time period or permanent. In some embodiments, treating can improve the likelihood of a complete response (iCR), a partial response (iPR) or stable disease (iSD) (e.g., as measured at 3 months, 6 months or 12 months, or at a later time), e.g., as compared to a control group not receiving the treatment. In some embodiments, treating can result in overall reduction in unconfirmed progressive disease (iUPD) or confirmed progressive disease (iCPD) (e.g., as measured at 3 months, 6 months or 12 months, or at a later time), e.g., as compared to a baseline prior to initiation of treatment. The reduction in iUPD or iCPD can be temporary over a certain time period or permanent. In some embodiments, treating can result in greater likelihood of overall reduction in unconfirmed progressive disease (iUPD) or confirmed progressive disease (iCPD) (e.g., as measured at 3 months, 6 months or 12 months, or at a later time), e.g., as compared to a control group not receiving the treatment. In some embodiments, treating can result in overall reduced number of new lesions according to iRECIST criteria, as compared to a control group not receiving the treatment or as compared to a baseline prior to initiation of the treatment (e.g., as measured at 3 months, 6 months or 12 months, or at a later time). The reduction in lesions can be temporary over a certain time period or permanent.

Response to treatment can also be characterized by one or more of immunophenotype in blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), PDL-1 expression (e.g., by immunohistochemistry), mismatch repair status, or tumor markers relevant for the disease (e.g., as measured at 3 months, 6 months or 12 months, or at a later time). Non-limiting examples of such tumor markers include Ca15-3, CA-125, CEA, CA19-9, alpha fetoprotein. These parameters can either be compared to baseline levels prior to initiation of treatment or can be compared to a control group not receiving the treatment.

In some embodiments, treating can result in changes in levels of immune cells and immune cell markers in the blood or in tumors, e.g., can result in immune activation. Such changes can be measured in patient blood and tissue samples using methods known in the art, such as multiplex flow cytometry and multiplex immunohistochemistry. For example, a panel of phenotypic and functional PBMC immune markers can be assessed at baseline prior to commencement of the treatment and at various time point during treatment. Table A lists non-limiting examples of markers useful for these assessment methods. Flow cytometry (FC) is a fast and highly informative method of choice technology to analyze cellular phenotype and function, and has gained prominence in immune phenotype monitoring. It allows for the characterization of many subsets of cells, including rare subsets, in a complex mixture such as blood, and represents a rapid method to obtain large amounts of data. Advantages of FC are high speed, sensitivity, and specificity. Standardized antibody panels and procedures can be used to analyze and classify immune cell subtypes. Multiplex IHC is a powerful investigative tool which provides objective quantitative data describing the tumor immune context in both immune subset number and location and allows for multiple markers to be assessed on a single tissue section. Computer algorithms can be used to quantify IHC-based biomarker content from whole slide images of patient biopsies, combining chromogenic IHC methods and stains with digital pathology approaches.

TABLE A
PBMC phenotyping markers

| PBMC phenotyping markers | | PBMC phenotyping markers | |
|---|---|---|---|
| CD3 | Total T cells | CD16 | NK cells |
| CD4 | CD4+ T cells | CD11b | Monocytes/macrophages |
| CD8 | CD8+ T cells | CD11c | Monocytes/macrophages, |
| | | | DCs |
| CD25 | Tres activation | CD14 | Monocyte subsets, |
| | | | macrophages |
| CD27 | T cell maturation; B cell | CD33 | Total myeloid cells |
| | nalve/mernory | | |
| CD38 | T cell maturation; B cell | FceR1 a | Antigen presenting DC |
| | naive/memory | | cells |
| CD45RA | Naive/memory cells | CD19 | Total B cells |
| CD45RO | Naive/memory cells | T-bet | T cells subsets |
| CD56 | NKT/NK cells (I cell | gdTCR | Gamma delta T cells |
| | subset) | | |
| CD127 | T cell subsets | CO274 (PDL-1) | Checkpoint |
| CD152 (CITA-4) | Checkpoint | Tim-3 | Checkpoint |
| CD279 (PD4) | Checkpoint | TCRVa24-Ja18 | iNKT cells |
| FoxP3 | Treg cells | live/dead | General |
| HLA-DR | Activation/Antigen | CD45 | General |
| | presentation | | |

Accordingly, in some embodiments, treating results in modulation of immune activation markers such as those in Table A, e.g., treating results in one or more of (1) an increase in more CD8 cells in plasma or tumor tissue, (2) a reduction in T regulatory cells (Tregs) in plasma or tumor tissue, (3) an increase in M1 macrophages in plasma or tumor tissue and (4) a decrease in MDSCs in plasma or tumor tissue, and (5) a decrease in M2 macrophages in plasma or tumor tissue (e.g., as measured at 3 months, 6 months or 12 months, or at a later time). In some embodiments, the markers that are assessed using the techniques described above or known in the art are selected from CD4, CD8 CD14, CD11b/c, and CD25. These parameters can either be compared to baseline levels prior to initiation of treatment or can be compared to a control group not receiving the treatment.

In some embodiments, treating as described herein results in changes in proinflammatory and anti-inflammatory cytokines. In some embodiments, treating as described herein results in one or more of (1) increased levels of IFNgamma in plasma or tumor tissue; (2) increased levels of TNFalpha in plasma or tumor tissue; (3) decreased levels of IL-10 in plasma or tumor tissue (e.g., as measured at 3 months, 6 months or 12 months, or at a later time). These parameters can either be compared to baseline levels prior to initiation of treatment or can be compared to a control group not receiving the treatment.

In some embodiments, changes in cytokines or immune cells may be assessed between a pre dose 1 tumor biopsy and repeat biopsy conducted at a feasible time. In some embodiments, changes in cytokines or immune cells may be assessed between 2 repeat biopsies. In some embodiments, treating results in a change one or more of in soluble galectin-9 levels in blood (serum or plasma), or in galectin-9 tumor tissue expression levels and pattern of expression by immunohistochemistry (tumor, stroma, immune cells), (e.g., as measured at 3 months, 6 months or 12 months, or at a later time). In some embodiments, treating results in a decrease of one or more of soluble galectin-9 levels in blood (serum or plasma), or in galectin-9 tumor tissue expression levels and pattern of expression by immunohistochemistry (tumor, stroma, immune cells) decrease. (e.g., as measured at 3 months, 6 months or 12 months, or at a later time). These galectin-9 levels can either be compared to baseline levels prior to initiation of treatment or can be compared to a control group not receiving the treatment.

In some embodiments, treating results in a change in PDL-1 expression, e.g., as assessed by immunohistochemistry. In some embodiments, treatments results in a change in one or more tumor markers (increase or decrease) relevant for the disease (e.g., as measured at 3 months, 6 months or 12 months, or at a later time). Non-limiting examples of such tumor markers include Ca15-3, CA-125, CEA, CA19-9, alpha fetoprotein. These parameters can either be compared to baseline levels prior to initiation of treatment or can be compared to a control group not receiving the treatment.

In some embodiments, treating results in improved quality of life and symptom control as compared to baseline prior to initiation of treatment or as compared to a control group not receiving the treatment (e.g., as measured at 3 months, 6 months or 12 months, or at a later time). In some embodiments, improvements can be measured on the ECOG scale described in Example 1 herein.

In any of the above embodiments, treating may comprise administering an anti-Galectin-9 antibody described herein alone or in combination with a checkpoint inhibitor therapy, e.g., an anti-PD-1 antibody. In some embodiments, the disclosure provides methods for treating a solid tumor in a subject, including a human subject, comprising administering to the subject a therapeutically effective amount of an anti-Galectin-9 antibody as disclosed herein. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for improving an overall response, e.g., according to RECIST 1.1. criteria (e.g., as measured at 3 months, 6 months or 12 months, or at a later time), in a subject, including a human subject, comprising administering to the subject a therapeutically effective amount of an anti-Galectin-9 antibody as disclosed herein. RECIST 1.1. criteria can either be compared to baseline levels prior to initiation of treatment or can be compared to a control group not receiving the treatment. In some embodiments, the disclosure provides methods for achieving a complete response, a partial response or stable disease (e.g., as measured at 3 months, 6 months or 12 months, or at a later time), the methods comprising administering to the subject a therapeutically effective amount of an anti-Galectin-9 antibody as disclosed herein. These responses can be temporary over a certain time period or permanent and can either be compared to baseline levels prior to initiation of treatment or can be compared to a control group not receiving the treatment.

In some embodiments, the methods can improve the likelihood of a complete response, a partial response or stable disease (e.g., as measured at 3 months, 6 months or 12 months, or at a later time; and being either temporary or permanent), e.g., as compared to a control group not receiving the treatment. In some embodiments, the disclosure provides methods for attenuating disease progression or reducing progressive disease (e.g., as measured at 3 months, 6 months or 12 months, or at a later time), e.g., as compared to a control group not receiving the treatment or as compared to baseline prior to initiation of the treatment, the method comprising administering to the subject a therapeutically effective amount of an anti-Galectin-9 antibody as disclosed herein. The attenuation or reduction can be temporary over a certain time period or permanent. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for reducing or maintaining tumor size in a subject, including a human subject, (e.g., as measured at 3 months, 6 months or 12 months, or at a later time) either permanently or over a minimum time period, relative to a baseline tumor size prior to initiation of the treatment in the subject, the method comprising administering to the subject a therapeutically effective amount of an anti-Galectin-9 antibody as disclosed herein. In some embodiments, the disclosure provides methods for improving the likelihood of reducing or maintaining tumor size in a subject, including a human subject, either permanently or over a minimum time period, (e.g., as measured at 3 months, 6 months or 12 months, or at a later time) e.g., as compared to a control group not receiving the treatment. In some embodiments, the disclosure provides methods for reducing or maintaining a tumor burden, in a subject, including a human subject (e.g., as measured at 3 months, 6 months or 12 months, or at a later time), as compared to baseline levels prior to initiation of the treatment or as compared to a control group not receiving the treatment, the methods comprising administering to the subject a therapeutically effective amount of an anti-Galectin-9 antibody as disclosed herein. In some embodiments, the disclosure provides methods for increasing the likelihood of reducing or maintaining a tumor burden (e.g., as measured at 3 months, 6 months or 12 months, or at a later time), e.g., as compared to a control group not receiving the treatment, the methods comprising administering to the subject a therapeutically effective amount of an anti-Galectin-9 antibody as disclosed herein. Accordingly, in some embodiments, tumor size and/or burden is measured in regularly scheduled restaging scans (e.g., CT with contrast, MRI with contrast, PET-CT (diagnostic CT) and/or X-ray). In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for increasing the time to disease progression or increasing the time of progression free survival (e.g., as measured at 3 months, 6 months or 12 months, or at a later time) in a subject, including a human subject, as compared to a control group that does not receive the treatment, the methods comprising administering to the subject a therapeutically effective amount of an anti-Galectin-9 antibody as disclosed herein. The methods can result in either permanent progression free survival or progression free survival over a certain amount of time. In some embodiments, the disclosure provides methods for increasing the likelihood of progression free survival (either permanent progression free survival or progression free survival over a certain amount of time (e.g., as measured at 3 months, 6 months or 12 months, or at a later time) as compared to a control group that does not receive the treatment. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for improving an overall response (iOR), e.g., according to iRECIST criteria (e.g., as measured at 3 months, 6 months or 12 months, or at a later time), in a subject, including a human subject, comprising administering to the subject a therapeutically effective amount of an anti-Galectin-9 antibody as disclosed herein. In some embodiments, the disclosure provides methods for achieving a “immune” complete response (iCR), a partial response (iPR) or stable disease (iSD) (e.g., as measured at 3 months, 6 months or 12 months, or at a later time), the methods comprising administering to the subject a therapeutically effective amount of an anti-Galectin-9 antibody as disclosed herein. In some embodiments, the methods can improve the likelihood of a “immune” complete response (iCR), a partial response (iPR) or stable disease (iSD) (e.g., as measured at 3 months, 6 months or 12 months, or at a later time). In some embodiments, the disclosure provides methods for attenuating disease progression or reducing progressive disease, e.g., reducing unconfirmed progressive disease (iUPD) or reducing confirmed progressive disease (iCPD)) (e.g., as measured at 3 months, 6 months or 12 months, or at a later time), the method comprising administering to the subject a therapeutically effective amount of an anti-Galectin-9 antibody as disclosed herein. Any of these above mentioned iRECIST criteria can either be compared to baseline levels prior to initiation of treatment or can be compared to a control group not receiving the treatment and response can be temporary over a certain time period or permanent. In some embodiments, the disclosure provides methods for increasing the likelihood of overall reduction in unconfirmed progressive disease (iUPD) or confirmed progressive disease (iCPD) (e.g., as measured at 3 months, 6 months or 12 months, or at a later time), in a subject, including a human subject, e.g., as compared to a control group not receiving the treatment, the methods comprising administering to the subject a therapeutically effective amount of an anti-Galectin-9 antibody as disclosed herein. In some embodiments, the disclosure provides methods for reducing the number of new lesions in a subject, including a human subject, according to iRECIST criteria (e.g., as measured at 3 months, 6 months or 12 months, or at a later time), the methods comprising administering to the subject a therapeutically effective amount of an anti-Galectin-9 antibody as disclosed herein. Reduced number of lesions can either be relative to baseline levels prior to initiation of treatment or relative to a control group not receiving the treatment, and the reduction can be temporary over a certain time period or permanent. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods of modulating an immune response in a subject. As used herein, the term “immune response” includes T cell-mediated and/or B cell-mediated immune responses that are influenced by modulation of immune cell activity, for example, T cell activation. In one embodiment of the disclosure, an immune response is T cell mediated. As used herein, the term “modulating” means changing or altering, and embraces both upmodulating and downmodulating. For example “modulating an immune response” means changing or altering the status of one or more immune response parameter(s). Exemplary parameters of a T cell mediated immune response include levels of T cells (e.g., an increase or decrease in effector T cells) and levels of T cell activation (e.g., an increase or decrease in the production of certain cytokines). Exemplary parameters of a B cell mediated immune response include an increase in levels of B cells, B cell activation and B cell mediated antibody production.

When an immune response is modulated, some immune response parameters may decrease and others may increase. For example, in some instances, modulating the immune response causes an increase (or upregulation) in one or more immune response parameters and a decrease (or downregulation) in one or more other immune response parameters, and the result is an overall increase in the immune response, e.g., an overall increase in an inflammatory immune response. In another example, modulating the immune response causes an increase (or upregulation) in one or more immune response parameters and a decrease (or downregulation) in one or more other immune response parameters, and the result is an overall decrease in the immune response, e.g., an overall decrease in an inflammatory response. In some embodiments an increase in an overall immune response, i.e., an increase in an overall inflammatory immune response, is determined by a reduction in tumor weight, tumor size or tumor burden or any RECIST or iRECIST criteria described herein. In some embodiments an increase in an overall immune response is determined by increased level(s) of one or more proinflammatory cytokine(s), e.g., including two or more, three or more, etc or a majority of proinflammatory cytokines (one or more, two or more, etc or a majority of anti-inflammatory and/or immune suppressive cytokines and/or one or more of the most potent anti-inflammatory or immune suppressive cytokines either decrease or remain constant). In some embodiments an increase in an overall immune response is determined by increased levels of one or more of the most potent proinflammatory cytokines (one or more anti-inflammatory and/or immune suppressive cytokines including one or more of the most potent cytokines either decrease or remain constant). In some embodiments an increase in an overall immune response is determined by decreased levels of one or more, including a majority of, immune suppressive and/or anti-inflammatory cytokines (the levels of one or more, or a majority of, proinflammatory cytokines, including e.g., the most potent proinflammatory cytokines, either increase or remain constant). In some embodiments, an increase in an overall immune response is determined by increased levels of one or more of the most potent anti-inflammatory and/or immune suppressive cytokines (one or more, or a majority of, proinflammatory cytokines, including, e.g., the most potent proinflammatory cytokines either increase or remain constant). In some embodiments an increase in an overall immune response is determined by a combination of any of the above. Also, an increase (or upregulation) of one type of immune response parameter can lead to a corresponding decrease (or downregulation) in another type of immune response parameter. For example, an increase in the production of certain proinflammatory cytokines can lead to the downregulation of certain anti-inflammatory and/or immune suppressive cytokines and vice versa.

In some embodiments, the disclosure provides methods for modulating an immune response (e.g., as measured at 3 months, 6 months or 12 months, or at a later time) in a subject, including a human subject, comprising administering to the subject a therapeutically effective amount of an anti-Galectin-9 antibody as disclosed herein. In some embodiments, the disclosure provides methods for modulating levels of immune cells and immune cell markers, including but not limited to those described herein in Table A, e.g., as compared to baseline levels prior to initiation of treatment, or as compared to a control group not receiving a treatment, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory immune cells and/or down regulation of immune-suppressive immune cells. In some embodiments, the disclosure provides methods for modulating levels of immune cells, wherein the modulating encompasses one or more of (1) increasing CD8 cells in plasma or tumor tissue, (2) reducing Tregs in plasma or tumor tissue, (3) increasing M1 macrophages in plasma or tumor tissue and (4) decreasing MDSC in plasma or tumor tissue, and (5) decreasing in M2 macrophages in plasma or tumor tissue, and wherein the methods comprise administering to the subject a therapeutically effective amount of an anti-Galectin-9 antibody as disclosed herein. In some embodiments, the markers to assess levels of such immune cells include but are not limited to CD4, CD8 CD14, CD11b/c, and CD25. In some embodiments, the disclosure provides methods for modulating levels of proinflammatory and immune suppressive cytokines (e.g., as measured at 3 months, 6 months or 12 months, or at a later time), e.g., as compared to baseline levels prior to initiation of treatment, or as compared to a control group not receiving a treatment, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory cytokines and/or down regulation of immune-suppressive cytokines. In some embodiments, the disclosure provides methods for modulating levels of cytokines cells, wherein the modulating encompasses one or more of (1) increasing levels of IFNgamma in plasma or tumor tissue; (2) increasing levels of TNFalpha in plasma or tumor tissue; (3) decreasing levels of IL-10 in plasma or tumor tissue. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for changing one or more of soluble galectin-9 levels in blood (serum or plasma), or in galectin-9 tumor tissue expression levels and pattern of expression by immunohistochemistry (tumor, stroma, immune cells) (e.g., as measured at 2 weeks, 4 weeks, 1 month, 3 months, 6 months or 12 months, or at a later time), comprising administering to the subject a therapeutically effective amount of an anti-Galectin-9 antibody as disclosed herein. In some embodiments of the methods, one or more of soluble galectin-9 levels in blood (serum or plasma), or in galectin-9 tumor tissue expression levels and pattern of expression by immunohistochemistry (tumor, stroma, immune cells) remain unchanged. In some embodiments, the methods provided herein decrease one or more of soluble galectin-9 levels in blood (serum or plasma), or in galectin-9 tumor tissue expression levels and pattern of expression by immunohistochemistry (tumor, stroma, immune cells) (e.g., e.g., as measured at 2 weeks, 4 weeks, 1 month, 3 months, 6 months or 12 months, or at a later time). Galectin-9 levels can either be compared to baseline levels prior to initiation of treatment or can be compared to a control group not receiving the treatment. In some embodiments, treating results in a change in PDL-1 expression, e.g., by immunohistochemistry. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for changing PDL-1 expression, e.g., as assessed by immunohistochemistry (e.g., as measured at 2 weeks, 4 weeks, 1 month, 3 months, 6 months or 12 months, or at a later time), comprising administering to the subject a therapeutically effective amount of an anti-Galectin-9 antibody as disclosed herein. In some embodiments of the methods, PDL-1 expression, e.g., as assessed by immunohistochemistry, remains unchanged. PD-L1 levels can either be compared to baseline levels prior to initiation of treatment or can be compared to a control group not receiving the treatment. In some embodiments, the methods provided herein decrease PDL-1 expression, e.g., as assessed by immunohistochemistry. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for changing one or more tumor markers (increasing or decreasing) relevant for the disease (e.g., as measured at 2 weeks, 4 weeks, 1 month, 3 months, 6 months or 12 months, or at a later time), comprising administering to the subject a therapeutically effective amount of an anti-Galectin-9 antibody as disclosed herein. In some embodiments of the methods, one or more tumor markers (increasing or decreasing) relevant for the disease, remain unchanged. Non-limiting examples of such tumor markers include Ca15-3, CA-125, CEA, CA19-9, alpha fetoprotein. Levels of tumor markers can either be compared to baseline levels prior to initiation of treatment or can be compared to a control group not receiving the treatment. In some embodiments, the methods provided herein decrease the occurrence of one or more tumor markers relevant for the disease. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the disclosure provides methods for improving quality of life and/or improving symptom control (e.g., as measured at 1 month, 3 months, 6 months or 12 months, or at a later time) in a subject, including a human subject, comprising administering to the subject a therapeutically effective amount of an anti-Galectin-9 antibody as disclosed herein. in improved quality of life and symptom control as compared to baseline prior to initiation of treatment or as compared to a control group not receiving the treatment. The improvements in quality of life can be temporary over a certain time period or permanent. In some embodiments, improvements can be measured on the ECOG scale. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor.

In some embodiments, the antibodies described herein, e.g., G9.2-17, are administered to a subject in need of the treatment at an amount sufficient to inhibit the activity of Galectin-9 (and/or Dectin-1 or TIM-3 or CD206) in immune suppressive immune cells in a tumor by at least 20% (e.g., 30%, 40%, 50%, 60%, 70%, 80%, 90% or greater) in vivo. In other embodiments, the antibodies described herein, e.g, G9.2-17, are administered in an amount effective in reducing the activity level of Galectin-9 (and/or Dectin-1 or TIM-3 or CD206) in immune suppressive immune cells in a tumor by at least 20% (e.g., 30%, 40%, 50%, 60%, 70%, 80%, 90% or greater) (as compared to levels prior to treatment or in a control subject). In some embodiments, the antibodies described herein, e.g., G9.2-17, are administered to a subject in need of the treatment at an amount sufficient to promote M1-like programming in TAMs by at least 20% (e.g., 30%, 40%, 50%, 60%, 70%, 80%, 90% or greater) in vivo (as compared to levels prior to treatment or in a control subject).

Conventional methods, known to those of ordinary skill in the art of medicine, can be used to administer the pharmaceutical composition to the subject, depending upon the type of disease to be treated or the site of the disease. In some embodiments, the anti-Galectin-9 antibody can be administered to a subject by intravenous infusion.

Injectable compositions may contain various carriers such as vegetable oils, dimethyllactamide, dimethylformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, the anti-Galectin-9 antibodies described herein are be used as a monotherapy for treating the target cancer disclosed herein, i.e., free of other anti-cancer therapy concurrently with the therapy using the anti-Galectin-9 antibody.

In other embodiments, the treatment method further comprises administering to the subject an inhibitor of a checkpoint molecule, for example, PD-1. Examples of PD-1 inhibitors include anti-PD-1 antibodies, such as pembrolizumab, nivolumab, tislelizumab and cemiplimab. Such checkpoint inhibitors can be administered simultaneously or sequentially (in any order) with the anti-Galectin-9 antibody according to the present disclosure. In some embodiments, the checkpoint molecule is PD-L1. Examples of PD-L1 inhibitors include anti-PD-L1 antibodies, such as durvalumab, avelumab, and atezolizumab. In some embodiments, the checkpoint molecule is CTLA-4. An example of a CTLA-4 inhibitor is the anti-CTLA-4 antibody ipilimumab. In some embodiments, the inhibitor targets a checkpoint molecule selected from CD40, GITR, LAG-3, OX40, TIGIT and TIM-3.

In some embodiments, the anti-Galectin-9 antibody improves the overall response, e.g., at 3 months, relative to a regimen comprising the inhibitor of the checkpoint molecule (e.g., anti-PD1, for example, nivolumab) alone.

In some embodiments, the anti-PD-1 antibody is PD-1 is nivolumab, and the method described herein comprises administration of nivolumab to the subject at a dose of 240 mg intravenously once every two weeks.

In some embodiments, the antibody that binds PD-1 is administered using a flat dose. In some embodiments, the antibody that binds PD-1 is nivolumab, which is administered to the subject at a dose of 480 mg once every 4 weeks. In some embodiments, the antibody that binds PD-1 is prembrolizumab, which is administered at a dose of 200 mg once every 3 weeks. In some embodiments, the antibody that binds PD-1 is cemiplimab. In some embodiments, the antibody that binds PD-1 is cemiplimab. In some embodiments, the methods described herein comprise administration of cemiplimab to the subject at a dose of 350 mg intravenously once every 3 weeks. In some embodiments, the antibody that binds PD-1 is Tislelizumab. In some embodiments, the methods described herein comprise administration of Tislelizumab to the subject at a dose of 200 mg intravenously once every 3 weeks.

In some embodiments, the antibody that binds PD-L1 is administered using a flat dose. In some embodiments, the antibody that binds PD-L1 is Atezolizumab. In some embodiments, the methods described herein comprise administration of Atezolizumab to the subject at a dose of 1200 mg intravenously once every 3 weeks. In some embodiments, the antibody that binds PD-L1 is Avelumab. In some embodiments, the methods described herein comprise administration of Avelumab to the subject at a dose of 10 mg/kg intravenously every 2 weeks. In some embodiments, the antibody that binds PD-1 is Durvalumab. In some embodiments, the methods described herein comprise administration of Durvalumab to the subject at a dose of 1500 mg intravenously every 4 weeks.

In specific examples, any of the methods disclosed herein comprise (i) administering to a human patient having a target solid tumor as disclosed herein (e.g., pancreatic ductal adenocarcinoma (PDA or PDAC), CRC, HCC, or CCA) any of the anti-Galectin-9 antibodies disclosed herein (e.g., G9.2-17 or the antibody having the heavy chain of SEQ ID NO:19 and the light chain of SEQ ID NO:5) at a dose of about 1 to about 32 mg/kg (e.g., about 3 mg/kg or about 15 mg/kg) once every two weeks; and (ii) administering to the human patient an effective amount of an anti-PD-1 antibody (e.g., nivolumab, prembrolizumab, Tislelizumab, or cemiplimab, durvalumab, avelumab, and atezolizumab). In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the solid tumor is selected from pancreatic adenocarcinoma (PDA), colorectal cancer (CRC), hepatocellular carcinoma (HCC), or cholangiocarcinoma (CCA), and in some embodiments, the solid tumor is a metastatic tumor. When nivolumab is used, a suitable dosing schedule can be about 480 mg once every 4 weeks. When prembrolizumab is used, a suitable dosing schedule can be about 200 mg once every 3 weeks. When cemiplimab is used, a suitable dosing schedule can be about 350 mg intravenously once every three weeks. When Tislelizumab is used, a suitable dosing schedule can be about 200 mg intravenously once every 3 weeks. In some embodiments an anti-PD-L1 antibody is used instead of an anti-PD-1 antibody. When Atezolizumab is used, a suitable dosing schedule can be about 1200 mg intravenously once every 3 weeks. When Avelumab is used, a suitable dosing schedule can be about 10 mg/kg intravenously every 2 weeks. When Durvalumab is used, a suitable dosing schedule can be about 1500 mg intravenously every 4 weeks.

Without being bound by theory, it is thought that anti-Galectin-9 antibodies, through their inhibition of Dectin-1, can reprogram immune responses against tumor cells via, e.g., inhibiting the activity of γδ T cells infiltrated into tumor microenvironment, and/or enhancing immune surveillance against tumor cells by, e.g., activating CD4+ and/or CD8+ T cells. Thus, combined use of an anti-Galectin-9 antibody and an immunomodulatory agent such as those described herein would be expected to significantly enhance anti-tumor efficacy.

In some embodiments, the methods are provided, the anti-Galectin-9 antibody is administered concurrently with a checkpoint inhibitor. In some embodiments, the anti-Galectin-9 antibody is administered before or after a checkpoint inhibitor. In some embodiments, the checkpoint inhibitor is administered systemically. In some embodiments, the checkpoint inhibitor is administered locally. In some embodiments, the checkpoint inhibitor is administered by intravenous administration, e.g., as a bolus or by continuous infusion over a period of time, by intramuscular, intraperitoneal, intracerobrospinal, subcutaneous, intra-arterial, intra-articular, intrasynovial, intrathecal, intratumoral, oral, inhalation or topical routes. In one embodiment, the checkpoint inhibitor is administered to the subject by intravenous infusion.

In any of the method embodiments described herein, the anti-galectin-9 antibody can be administered (alone or in combination with an anti-PD1 antibody) once every 2 weeks for one cycle, once every 2 weeks for two cycles, once every 2 weeks for three cycles, once every 2 weeks for four cycles, or once every 2 weeks for more than four cycles. In some embodiments, the treatment is 1 to 3 months, 3 to 6 months, 6 to 12 months, 12 to 24 months, or longer. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

A subject being treated by any of the anti-galectin-9 antibodies disclosed herein (e.g., G9.2-17), either alone or in combination with a checkpoint inhibitor (e.g., an anti-PD-1 or anti-PD-L1 antibody) as disclosed herein may be monitored for occurrence of adverse effects (for example, severe adverse effects). Exemplary adverse effects to monitor are provided in Example 1 below. If occurrence of adverse effects is observed, treatment conditions may be changed for that subject. For example, the dose of the anti-galectin-9 antibody may be reduced and/or the dosing interval may be extended. Suitability and extent of reduction may be assessed by a qualified clinician. In one specific example, a reduction level as per clinician's assessment or at least by 30% is implemented. If required, one more dose reduction by 30% of dose level −1 is implemented (dose level −2). Alternatively or in addition, the dose of the checkpoint inhibitor can be reduced and/or the dosing interval of the checkpoint inhibitor may be extended. In some instances (e.g., occurring of life threatening adverse effects), the treatment may be terminated.

Kits for Use in Treatment of Diseases Associated with Galectin-9

The present disclosure also provides kits for use in treating or alleviating a disease associated with Galectin-9, for example associated with Galectin-9 binding to a cell surface glycoprotein (e.g., Dectin-1, TIM3, CD206, etc.), or pathologic cells (e.g., cancer cells) expressing Galectin-9. Examples include solid tumors such as PDA, CRC, HCC, or cholangiocarcinoma, and others described herein and others described herein. Such kits can include one or more containers comprising an anti-Galectin-9 antibody, e.g., any of those described herein, and optionally a second therapeutic agent (e.g., a checkpoint inhibitor such as an anti-PD-1 antibody as disclosed herein) to be co-used with the anti-Galectin-9 antibody, which is also described herein.

In some embodiments, the kit can comprise instructions for use in accordance with any of the methods described herein. The included instructions can comprise a description of administration of the anti-Galectin-9 antibody, and optionally the second therapeutic agent, to treat, delay the onset, or alleviate a target disease as those described herein. In some embodiments, the kit further comprises a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Galectin-9 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease associated with Galectin-9 (e.g., Dectin-1, TIM-3, or CD206 signaling). In some embodiments, instructions are provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container may be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is an anti-Galectin-9 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. A Phase I-II Open Label Non-Randomized Study Using Anti-Galectin-9 Monoclonal Antibody Alone or in Combination with an Anti-PD1 Antibody in Patients with Metastatic Solid Tumors

Galectin-9 is a molecule overexpressed by many solid tumors, including those in pancreatic cancer, colorectal cancer, and hepatocellular carcinoma. Moreover, Galectin-9 is expressed on tumor-associated macrophages, as well as intra-tumoral immunosuppressive gamma delta T cells, thereby acting as a potent mediator of cancer-associated immunosuppression. As described herein, monoclonal antibodies targeting Galectin-9 (e.g., G9.2-17, IgG4) have been developed. Data have demonstrated that the G9.2-17 halts pancreatic tumor growth by 50% in orthotopic KPC models and extended the survival of KPC animals by more than double. Without wishing to be bound by theory, it is thought that the anti-Galectin-9 antibodies reverse the M2 to M1 phenotype, facilitating intra-tumoral CD8⁺ T cell activation. In additional, antibody G9.2-17 (IgG4) (having a heavy chain of SEQ ID NO:19 and a light chain of SEQ ID NO:15) has been found to synergize with anti-PD1.

The purpose of this Phase I/II multicenter study is to determine the safety, tolerability, maximum tolerated or maximum administered dose (MTD), and objective tumor response after three months of treatment in subjects having metastatic solid tumors, e.g., pancreatic adenocarcinoma (PDA), colorectal cancer (CRC), hepatocellular carcinoma (HCC), or cholangiocarcinoma (CCA). The study also examines progression-free survival (PFS), the duration of response (by RESIST), disease stabilization, the proportion of subjects alive at 3, 6, and 12 months, as well as pharmacokinetic (PK) and pharmacodynamics (PD) parameters. Subjects undergo pre- and post-treatment biopsies, as well as PET-CT imaging pre-study and once every 8 weeks for the duration of the study. In addition, immunological endpoints, such as peripheral and intra-tumoral T cell ratios, T cell activation, macrophage phenotyping, and Galectin-9 serum levels are examined. The study is performed under a master study protocol, and the study lasts for 12-24 months.

Further, preclinical proof of concept data demonstrate that G9.2-17 (IgG4) (a.k.a., G9.2-17 IgG4) reduces pancreatic tumor growth by up to 50% in orthotopic ((LSL-Kras (G12D/±); LSL-Trp53 (R172H/+); Pdx-1-Cre)-pancreatic ductal adenocarcinoma) KPC models and B16F10 melanoma, subcutaneous model, as a single agent. Blocking galectin-9 also extends survival of KPC animals. Mechanistically, targeting galectin-9 facilitates intra-tumoral effector T cell activation. There is an indication of synergy between G9.2-17antibody and anti-PD-1 in vivo. Namely, in the B16F10 melanoma model, a significantly greater increase in intratumoral CD8+ T cells in groups treated with anti-galectin-9 antibody and anti-PD-1 was observed, as relative to groups treated with either single agent alone. In non GLP toxicity studies, G9.2-17 (IgG4) is safe in rodents and cynomolgus monkeys at doses up to and inclusive of 100 mg/kg in rodents and 300 mg/kg in monkeys. This Phase 1a/lb investigational trial aims at evaluating safety and tolerability of the maximally tolerated dose, PK, PD, efficacy response outcome, disease control, and survival at 3, 6 and 12 months, and other exploratory parameters.

This Phase 1a/lb investigational trial evaluates safety and tolerability of the maximum tolerated dose (or maximum administered dose), PK, PD, immunogenicity, efficacy response outcome, patient survival, and other exploratory parameters. While pancreatic cancer, colorectal cancer and cholangiocarcinoma are the planned expansion cohorts, the dose finding part of the clinical trial is open for all comers with metastatic solid tumors, beyond the above noted tumor types. Other cancer types beyond PDAC, CRC and CCA, may benefit from anti-galectin-9 treatment, and while not currently prioritized for expansion cohorts, may demonstrate meaningful clinical benefit and mechanistic rationale in the dose escalation part, to warrant dedicated expansion cohorts. Furthermore, expansion cohorts in CRC and CCA are planned for single agent G9.2-17 IgG4, as well as G9.2-17 IgG4 in combination with an approved anti-PD-1 agent for patients who have failed at least one prior line of treatment in the metastatic setting and are otherwise eligible for the study.

Primary objectives include safety, tolerability, maximum tolerated dose (MTD), objective tumor response (ORR) at 3 months. Secondary objectives include progression free survival (PFS), duration of response by RECIST 1.1, disease stabilization, proportion alive at 3, 6 and 12 months as well as pharmacokinetic (PK) and pharmacodynamic parameters (PD).

Subject, disease, and all clinical and safety data are presented descriptively as means, medians, or proportions, with appropriate measures of variance (e.g., 95% confidence interval range). Waterfall and Swimmers plots are used to graphically present the ORR and duration of responses for subjects for each study arm, within each disease site, as described below. Exploratory correlations analysis are also undertaken to identify potential biomarkers that may be associated with ORR. All statistical analyses are performed using SAS, version 9.2 (SAS, Cary, N.C.).

This study includes both monotherapy of G9.2-17 (IgG4) and combination of G9.2-17 and nivolumab. Doses of G9.2-17 may range from about 3 mg/kg to 15 mg/kg once every two weeks. The antibody is administered by intravenous infusion.

Study Objectives, Duration and Study Population are summarized in Table B.

TABLE B
Study Overview

| | |
|---|---|
| Study | Part 1 (Phase 1a) |
| Objectives | Primary Objective(s) |
| | Safety, tolerability, optimal biological dose (OBD), or maximum |
| | administered dose (MTD), recommended Phase 2 dose (RP2D) |
| | Secondary Obi ective(s) |
| | Pharmacokinetic (PK), pharmacodynamic (PD) parameters, immunogenicity |
| | Exploratory Objective(s) |
| | Exploratory end points for part 1, in addition to exploratory end points listed |
| | below: Objective Response Rate (ORR), disease control rate (DCR), |
| | progression free survival (PFS), patient survival at 6 and 12 months. |
| | Part 2 in CRC and CCA |
| | Primary Objective(s) |
| | Objective Response Rate (ORR) |
| | Secondary Objective(s) |
| | Progression free survival (PFS), disease control rate (DCR), duration and |
| | depth of response by RECIST 1.1, patient survival at 6 and 12 months, time |
| | to response, safety and tolerability |
| | Part 2 in PDAC |
| | Primary Objective(s) |
| | Progression free survival (PFS) at 6 months |
| | Secondary Objective(s) |
| | Objective Response Rate (ORR), disease control rate (DCR) at 3, 6 and 12 |
| | months, patient survival at 6 and 12 months, time to response, duration and |
| | depth of response by RECIST 1.1, safety and tolerability |
| | Exploratory Endpoints for All Study Parts: iRECIST criteria, |
| | immunophenotyping from blood and tumors, cytokine profile (serum), |
| | soluble galectin-9 levels in blood (serum or plasma), galectin-9 tumor tissue |
| | expression levels and pattern of expression by immunohistochemistry |
| | (tumor, stroma, immune cells), tumor mutational burden (TMB), PDL-1 |
| | expression by immunohistochemistry, mismatch repair status, tumor markers |
| | relevant for the disease, ctDNA - and correlation of these parameters with |
| | response. Time to response (TTR). Quality of life and symptom control. |
| | Part 3: Before proceeding to Part 3, the trial is revised to specify the Part 3 |
| | objectives, trial population, treatment, and statistical analysis plan. |
| Duration of | Study duration: 12-24 months |
| Treatment/ | Study drug administration continues until progression of disease, |
| Study | unacceptable toxicity or withdrawal from the study. Patients who discontinue |
| Participation | study drug prior to progressive disease are followed on study until the time |
| | of disease progression. |
| Study | Part 1: Patients with relapsed/refractory metastatic cancers, irrespective of |
| Population | tumor type, are eligible for the dose-finding study using the continual |
| | reassessment method (CRM) as described by O'Quigley (1990; Continual |
| | reassessment method: a practical design for phase 1 clinical trials in cancer; |
| | Biometrics. 1990 Mar;46(1):33-48.). |
| | Part 2: Expansion is envisaged in PDAC, CRC and CCA, or in tumor types |
| | where mode of action and/or an early efficacy signal are captured in Part 1. |
| | Combination with an approved anti-PD-1 agent is implemented for patients |
| | who have failed at least one prior line of treatment in the metastatic setting |
| | and are otherwise eligible for the study |
| Other Drugs | An approved Anti-PD-1 mAb; Anti-PD-1 mAb dose is determined |
| | depending on approved drug dose or is determined by initial IND; Mode of |
| | Administration: Intravenous infusion |
| Statistical | Subject, disease and all clinical safety data are presented descriptively as |
| Method | means, medians, or proportions, with appropriate measures of variance (i.e. |
| | 95% CI, range). Waterfall and Swimmers plots are used to graphically |
| | present the RR and duration of responses for patients for each study arm, |
| | within each disease site. |
| | All efficacy analyses are based on the Intention-to-treat (ITT) population |
| | unless otherwise specified. Survival curves for progression-free survival |
| | (PFS) and overall survival (OS) are generated by the method of Kaplan- |
| | Meier. Exploratory correlations analysis is also undertaken to identify |
| | potential biomarkers and other predictors that are potentially associated with |
| | ORR, PFS and OS. |
| Study | Incidence and severity of adverse evernts (AEs)/Common Terminology |
| Procedures and | Criteria adverse event (CTAEs) and Serious adverse events (SAEs), including |
| Evaluations | clinically significant changes in laboratory parameters, vital signs, |
| | performance status and electrocardiogram ( ECG). |
| | Incidence of DLTs, PK and PD |
| | Plasma PK parameters (e.g., AUC0-24h, Cmax, Tmax, estimated half- |
| | life); serum concentration vs. time profiles |
| | Objective response rate (complete response and partial response) (ORR) |
| | and clinical benefit rate (objective response and stable disease 3 months or |
| | longer); progression-free survival (PFS), overall survival (OS), disease |
| | control rate (DCR) |
| | Blood and tumor immunophenotyping, galectin-9 serum or plasma levels, |
| | and tissue expression levels and expression pattern within the tumor, stroma, |
| | immune cells compartments, time to response (TTR), and other biomarker |
| | analysis, CT (PET-CT)imaging, and other clinically relevant imaging. |
| Safety | In Part 1, the dose-escalation phase, dose escalation to the next cohort |
| Monitoring | proceeds following review of Cycle 1 of each cohort. Safety and available |
| | PK data is used to assess for a dose-limiting toxicity (DLT) in all patients of |
| | each cohort. As a safety precaution during dose escalation, new patients are |
| | entered and treated only after the first patient of each cohort has been treated |
| | with G9.2-17 IgG4 and at a minimum 7 days post-treatment has elapsed. |
| | Select DLT safety analysis for each patient will be performed following |
| | completion of Cycle 1. During the expansion phase, toxicities are monitored |
| | to review aggregate toxicity rate prior to each dose escalation. |

### Study Design

Patient population: Metastatic all corners in the 3+3 dose escalation Stage 1 (disclosed below) then expansion in PDA, CRC and CCA or in tumor types where mode of action and/or an early efficacy signal are captured in Stage 1.

Stage 1

A dose-finding study is to be conducted using a continuous reassessment method (CRM)—O'Quigley et al. (1990), a model-based design that informs how the dosage of anti-Gal9 antibody should be adapted for the next patient cohort based on past trial data. Stage 1 of the study is a 3+3 dose finding and safety when the anti-Galectin-9 antibody is administered as a single agent.

A one parameter power model is to be used to describe the relationship between the dose of G9.2-17 (IgG4) and the probability of observing a dose limiting toxicity (DLT). DLT is defined as a clinically significant non-hematologic adverse event or abnormal laboratory value assessed as unrelated to metastatic tumor disease progression, intercurrent illness, or concomitant medications and is related to the study drug and occurring during the first cycle on study that meets any of the following criteria:
All Grade 4 non-hematologic toxicities of any duration
All Grade 3 non-hematologic toxicities. Exceptions are as follows:
   Grade 3 nausea, vomiting and diarrhea that does not require hospitalization or TPN support and can be managed with supportive care to <grade 2 within 48 hours.
   Grade 3 electrolyte abnormalities that are corrected to <grade 2 within 24 hours.

DLT Period=One (1) cycle, i.e., two doses of the anti-Gal9 antibody on days 1 and 15 of each cycle.
Incidence and severity of AEs/CTAEs and SAEs, including clinically significant changes in laboratory parameters, vital signs & ECGs
Incidence of DLTs, PK and PD
Plasma PK parameters (e.g., AUC₀₋₂₄ₕ, Cₘₐₓ, Tₘₐₓ, estimated half-life); serum concentration vs. time profiles
Objective response rate (complete response and partial response) and clinical benefit rate (objective response and stable disease 3 months or longer); progression free survival (PFS), overall survival (OS), disease control rate (DCR)

Blood and tumor immunophenotyping, galectin-9 serum, plasma levels, and tissue expression levels and expression pattern of tumor, stroma, immune cells), time to response (TTR), and other biomarker analysis, CT (PET) imaging/other clinically indicated imaging modality.

The OBD is the largest dose that has an estimated probability of a DLT less than or equal to a target toxicity level (TTL) of 25%. Two patients at a time are to be dosed, with a maximum available sample size of 24. As a safety precaution, at each dose escalation, new patients will be entered and treated only after the first patient of each cohort has been treated with the anti-Gal9 antibody and at a minimum 7 days post-treatment has elapsed.

The dose range is shown in Table 2 below and the antibody is administered once every two weeks (Q2W) intravenously.

TABLE 2
Dosing by Cohort

| | | NO. OF | DOSE | | ADMINISTRATION |
|---|---|---|---|---|---|
| STAGE 1 | COHORT | SUBJECTS* | INCREASE^{A} | GALACTIN-9^{B} | AND DURATION** |
| Galactin-9 | COHORT | 3-6 | | Galectin-9^{B} | 1 administration |
| (single | 1 | | | 3 mg/kg | intravenously, every 2 |
| agent, | COHORT | 3-6 | 100% | Galectin-9^{B}, | weeks for 3 months |
| N = 15-30) | 2 | | | 6 mg/kg | |
| | COHORT | 3-6 | 67% | Galectin-9^{B} | |
| | 3 | | | 10.02 mg/kg | |
| | COHORT | 3-6 | 50% | Galectin-9^{B} | |
| | 4 | | | 15.03 mg/kg | |
| | COHORT | 3-6 | 40% | Galectin-9^{B} | |
| | 5 | | | 21.02 mg/kg | |
| | COHORT | 3-6 | 30% | Galectin-9^{B} | |
| | 6 | | | 27.33 mg/kg | |

^{A}If none of the first 3 patents experiences a dose limifng toxicity (DLT), another 3 patients are treated at the next higher dose level. However, if one of the three patients has a DLT, another 3 patients are treated at the same dose level. Dose escalation continues until at least 2 patients among the cohort of 3 to 6 patients experiences the DLT. The dose for stage II is the dose just below the level exhibiting the toxicity.
^{B}The respective trial arm is terminated when ≤ 1 patients respond

Dose escalation follow a modified Fibonacci sequence where the dose is increased by 100% of the preceding first dose, then followed by increases of 67%, 50%, 40%, and 30% of the preceding doses. If none of the first three patients experience a dose limiting toxicity (DLT), then another three subjects are treated at the next highest dose level. Alternatively, if one of the three subjects has a DLT, then another three subjects are treated at the same dose level. Dose escalation continues until at least two patients among the cohort of three to six patients experience a DLT.

In an alternative design, Stage 1 is to be completed when six consecutive patients have received the same dose and that dose will be identified as the OBD. A total of 5 dosage levels are to be evaluated within the CRM design.

1. Dose level 1=2 mg/kg

2. Dose level 2=4 mg/kg

3. Dose level 3=8 mg/kg

4. Dose level 4=12 mg/kg

5. Dose level 5=16 mg/kg

6. Dosing regimen: Q2W

7. Route of administration: Intravenous (IV)

Stage 2

Stage 2 of the study is a Simon's two-stage optimal design (six arms: pancreatic ductal adenocarcinoma (PDA), CRC, and Cholangiocarcinoma). The study investigates the use of the anti-Galectin-9 antibody alone (single agent arms of the study) and in conjunction with nivolumab (a 240 mg flat dose administered once every two weeks; 10 combination arms of the study). The dose of the anti-Galectin-9 antibody used is below the level found to exhibit toxicity in the Phase I stage.

In CRC and CCA, the anti-Gal9 antibody is to be tested as single agent. Alternatively, the anti-Gal9 antibody is to be tested in combination with an approved anti-PD-1 mAb (e.g., nivolumab, pembrolizumab, cemiplimab, or).

The optimal two-stage design is used to test the null hypothesis that the ORR≤5% versus the alternative that the ORR≥15% within the single agent arms. After testing the drug on 23 patients in the first stage, the respective trial arm is terminated if ≤1 patients respond. If the trial goes on to the second part of Simon's optimal design, a total of 56 patients are enrolled into each of the single agent arms. If the total number responding patients is ≤5, the drug within that arm is rejected. If ≥6 patients have an ORR at 3 months, the expansion cohort for that arm is activated. The above approach is applied to the single agent arms of the study.

For the IO combination arms (CRC and CCA), the starting dose of G9.2-17 IgG4 is one dose lower than the RP2D dose level (RP2D-1), identified in Part 1. To ensure patient safety, the Sponsor plans a safety run-in whereby the first 8 patients is dosed with the combination and that arm will be continued only if ≤2 patients develop a DLT, which is below the TTL of 25%. If 3 or more patients develop a DLT, the dose of G9.2-17 IgG4 will be reduced by a reduction level as per clinician's assessment or at least by 30% (dose level-1) If required, one more dose reduction by 30% of dose level −1 is allowed (dose level-2). No further dose reductions is allowed. Dose reduction to dose level −1 and −2 is allowed only if the investigator assesses that clinical benefit is being derived and may continue to be derived under dose reduced conditions.

For the anti-PD-1 mAb combination arms in CRC and CCA, the optimal two-stage design is also used to test the null hypothesis that the ORR≤10% versus the alternative that the ORR≥25%. After testing the combination on 18 patients in the first stage, the respective trial arm is terminated if ≤2 patients respond. If the trial goes on to the second part of Simon optimal design, a total of 43 patients is enrolled into each of the combination arms. If the total number of responding patients is ≤7, the combination within that arm is rejected. If ≥8 patients have an ORR at 3 months, the expansion cohort for that arm is activated.

Stage 3

Stage 3 includes expansion of cohorts where early efficacy signal has been detected. If a promising efficacy signal is identified within one of the six trial arms that is attributable to the tumor type, an expansion cohort is launched to confirm the finding. The sample size for each of the expansion arms is determined based on the point estimates determined in Stage 2, in combination with predetermined level of precision for the 95% confidence interval (95% CI) around the ORR.

Pre- and post-treatment biopsy samples are analyzed in this study, e.g., imaging PET-CT pre-study and Q6/8W, as clinically indicated. PK, PD, immunological end points include peripheral and intra-tumoral T cell ratios, T cell activation, macrophage phenotyping, Galectin-9 serum levels, and Galectin-9 tissue expression levels.

Dosing and Administration

G9.2-17 IgG4 is administered via intravenous (IV) infusion every two weeks (Q2W) until progression of disease, unacceptable toxicity, or withdrawal of consent in Part 1 and Part 2. Subjects who experience a dose-limiting toxicity may resume G9.2-17 IgG4 administration if the patient is experiencing clinical benefit, as per investigator's judgement and after a discussion with the Study Medical Monitor. Dose reduction of 30% or as per the clinical discretion of the investigator and with agreement of the Medical Monitor and the Sponsor. Dose reduction by 30% will considered dose level −1. The next dose reduction of 30% of the previous dose level will be considered dose level −2. No more than two such dose reductions are allowed.

Part 1: Subjects receive G9.2-17 IgG4alone in accordance with the CRM design.

Part 2: Subjects receive the RP2D of G9.2-17 IgG4 as a single agent or G9.2-17 IgG4 in combination with anti-PD-1 using the RP2D identified within Part 1. However, in the case of the combination arms, the first 8 patients are dosed and that arm is continued on if ≤2 patients develop a DLT, which is below the target toxicity level (TTL) of 30%. If more than 3 patients develop a DLT determined to be G9.2-17 IgG4 related and not related to the drug/regimen used in combination, then G9.2-17 IgG4 will be dose reduced to RP2D-1 dose level (30% dose reduction of G9.2-17 IgG4 or as per clinician's assessment).

### Study Objectives

(ii) Stage 1 (Phase 1a)
   Primary Objective(s): Safety, tolerability, optimal biological dose (OBD), maximum tolerated dose (MTD) or maximum administered dose (MAD), recommended Phase 2 dose (RP2D)
   Secondary Objective(s): Pharmacokinetic (PK), pharmacodynamic (PD) parameters, immunogenicity
   Exploratory end points for Stage 1: in addition to exploratory end points listed below: Objective Response Rate (ORR), disease control rate (DCR), progression free survival (PFS), patient survival at 6 and 12 months
(ii) Stage 2 and Stage 3 in CRC and CCA
   Primary Objective(s); Objective Response Rate (ORR)
   Secondary Objective(s): Progression free survival (PFS), disease control rate (DCR), duration and depth of response by RECIST 1.1, patient survival at 6 and 12 months, time to response, safety and tolerability
(iii) Stage 2 and Stage 3 in PDAC
   Primary Objective(s); Patient survival at 6 months
   Secondary Objective(s): Objective Response Rate (ORR), progression free survival (PFS), disease control rate (DCR) at 3, 6 and 12 months, patient survival at 6 and 12 months, duration and depth of response by RECIST 1.1, time to response, safety and tolerability
(iv) Exploratory Endpoints for All study Parts:
   iRECIST criteria, immunophenotyping from blood and tumors, cytokine profile (serum), soluble galectin-9 levels in blood (serum or plasma), galectin-9 tumor tissue expression levels and pattern of expression by immunohistochemistry (tumor, stroma, immune cells), multiplex immunohistochemistry, time to response (TTR), tumor mutational burden (TMB), PDL-1 expression by immunohistochemistry, mismatch repair status, tumor markers relevant for the disease—and correlation of these parameters with response. Quality of life and symptom control.

### Study Population

(i) Stage 1: Patients with relapsed/refractory metastatic cancers, irrespective of tumor type, will be eligible for the dose-finding study using the continual reassessment method (CRM) as described by O'Quigley (1990).

(ii) Stage 2: Expansion is envisaged in PDAC, CRC and CCA (planned), or in tumor types where mode of action and/or an early efficacy signal are captured in Stage 1.

(iii) Stage 3: The final and third part of the study allows for further expansion of cohorts from Stage 2 that demonstrate a minimum threshold for anti-tumor activity. The sample size for each of the expansion arms will be determined based on the point estimates determined in Stage 3, in combination with a predetermined level of precision for the 95% confidence interval (95% CI) around ORR/patient survival.

Patient Inclusion Criteria:

1. Written Informed Consent (mentally competent, ability to understand and willingness to sign the informed consent form)

2. Aged >18 years male or non-pregnant female

3. Histologically confirmed unresectable metastatic cancer

4. Able to comply with the study protocol, in the investigator's judgment

5. Life expectancy >3 months

6. Recent archival tumor sample available for biomarker analysis. Information must be available of therapies received since the biopsy specimen was obtained. Investigator's judgement will be used to determine whether the archival specimen as such is acceptable.

7. Galectin-9 tumor tissue expression levels assessed by IHC on an archival specimen(s), in accordance with inclusion criteria in point 5, is to be recorded if available.

8. Patient able and willing to undergo pre- and on/post-treatment biopsies. According to the investigator's judgment, the planned biopsies should not expose the patient to substantially increased risk of complications. Every effort is made that the same lesion is biopsied on repeat biopsies.

9. Measurable disease, according to RECIST v1.1. Note that lesions intended to be biopsied should not be target lesions.

10. Expected survival according to investigator's judgement >3 months

11. For Part 1: No available standard of care options, or patient has declined available and indicated standard of care therapy, or are not eligible for available and indicated standard of care therapy For Part 2: PDAC expansion cohort—received at least one line of systemic therapy in the metastatic cancer setting and for, patients who are either gemcitabine containing regimen naïve or at least 6 months out of having been treated using a gemcitabine containing regimen. CCR and CCA expansion cohorts—received at least one prior line of therapy in the metastatic setting.

12. Vaccination for COVID-19 is allowed before or during the study period. Information on timing and type of vaccine must be recorded.

13. Eastern Cooperative Oncology Group (ECOG) performance status 0-1/Karnofsky score >70 (please record both whenever possible)

14. MSI-H and MSS patients are to be allowed in Part 1 (Stage 1) of the study

15. Adequate hematologic and end organ function, defined by the following laboratory results obtained within 28 days prior to first dose of study drug treatment and within 72 hours before any consecutive dose of the study drug: neutrophil count ≥1×109/L, platelet count ≥100×109/L, for HCC in Part 1≥50×109/L. hemoglobin ≥8.5 g/dL without transfusion in the previous week, Creatinine ≤1.5 ULN, Creatinine clearance ≥30 mL/min, AST (SGOT)≤3×ULN (≤5×ULN when HCC or hepatic metastases are present), ALT (SGPT)≤3×ULN, (≤5×ULN when HCC or hepatic metastases present) Bilirubin ≤1.5×ULN (patients with known Gilbert's disease may have a bilirubin ≤3.0×ULN), Albumin ≥3.0 g/dLINR and PTT≤1.5×ULN; amylase and lipase ≤1.5×ULN.
If with previously diagnosed brain metastases, must have completed treatment for brain disease, either surgery or radiation therapy, 4 weeks or longer prior to screening, or have stable brain disease for at least 3 months before study start. Brain MRI is required in such cases to demonstrates no current evidence of progressive brain metastases and no new disease in the brain and/or leptomeningeal disease,
Patients must have discontinued steroids given for the management of brain metastases at least 28 days before study start.

16. No evidence of active infection and no serious infection within 4 weeks before study start

17. Women of child-bearing potential must have a negative pregnancy test within 72 hours prior to start of treatment. For women of childbearing potential: agreement to remain abstinent (refrain from heterosexual intercourse) or use of contraceptive methods that result in a failure rate of <1% per year during the treatment period and for at least 180 days after the last study treatment. A woman is of childbearing potential if she is post-menarchae, has not reached a postmenopausal state (≥12 continuous months of amenorrhea with no identified cause other than menopause), and has not undergone surgical sterilization (removal of ovaries and/or uterus). Examples of contraceptive methods with a failure rate of <1% per year include bilateral tubal ligation, male sterilization, hormonal contraceptives that inhibit ovulation, hormone-releasing intrauterine devices and copper intrauterine devices. The reliability of sexual abstinence should be evaluated in relation to the duration of the clinical trial and the preferred and usual lifestyle of the patient. Periodic abstinence (e.g., calendar, ovulation, symptom-thermal, or post ovulation methods) and withdrawal are not acceptable methods of contraception. Fertile men must practice effective contraceptive methods during the study, unless documentation of infertility exists.

18. Four (4) weeks or 5 half lives (whichever is shorter) since the last dose of anti-cancer therapy before the first G9.2-17 IgG4 administration

19. Continuation of bisphosphonate treatment (zolendronic acid) or denosumab for bone metastases which have been stable for at least 6 months before C1D1 is allowed.

20. For CCR and CCA expansion cohorts, at least one prior line of therapy in the metastatic setting is required.

Patient Exclusion Criteria:
1. Patient diagnosed with metastatic cancer of an unknown primary
2. Unwilling or unable to follow protocol requirements
3. Prior or current illicit drug addiction
4. Clinically significant, active uncontrolled bleeding, and any patients with a bleeding diathesis (e.g., active peptic ulcer disease). prophylactic or therapeutic use of anticoagulants is allowed.
5. Lactating females
6. Receiving any other investigational agents or participating in any other clinical trial involving another investigational agent for treatment of solid tumors within 4 weeks or 5 half-lives of the administered drug (whichever is shorter) prior to cycle 1, day 1 of the study or other investigational therapy or major surgery within 4 weeks from the date of consent, or planned surgery within 4 weeks from envisaged study start (this includes dental surgery).
7. Radiation therapy within 4 weeks of the first dose of study drug, except for palliative radiotherapy to a limited field, such as for the treatment of bone pain or a focally painful tumor mass.
8. Patients with fungating tumor masses Patients with locally advanced PDAC.
9. ≥CTCAE grade 3 toxicity (except alopecia and vitiligo) due to prior cancer therapy. Grade 4 immune-mediated toxicities with a prior checkpoint inhibitor. Grade 2 or Grade 3 pneumonitis or any other Grade 3 checkpoint inhibitor-related toxicity that led to immunotherapy treatment discontinuation.
10. History of second malignancy except those treated with curative intent more than five years previously without relapse or low likelihood of recurrence (for example non-melanotic skin cancer, cervical carcinoma in situ, prostate cancer or superficial bladder cancer)
11. Evidence of severe or uncontrolled systemic diseases, congestive cardiac failure >New York Heart Association (NYHA) class 2, Myocardial Infarction (MI) within 6 months or laboratory finding that in the view of the Investigator makes it undesirable for the patient to participate in the trial
12. Any medical condition that the Investigator considers significant to compromise the safety of the patient or that impairs the interpretation of G9.2-17 IgG4 toxicity assessment
13. Serious non-healing wound, active ulcer or untreated bone fracture
14. Uncontrolled pleural effusion, pericardial effusion, or ascites requiring recurrent drainage procedures
15. History of severe allergic, anaphylactic, or other hypersensitivity reactions to chimeric or humanized antibodies or fusion proteins
16. Significant vascular disease (e.g., aortic aneurysm requiring surgical repair or recent arterial thrombosis) within 6 months of Cycle 1 Day 1
17. History of pulmonary embolism, stroke or transient ischemic attack within 2 or 3 months prior to Cycle 1 Day
18. History of abdominal fistula or gastrointestinal perforation within 6 months prior to Cycle 1 Day 1
19. Active auto-immune disorder (except type I diabetes, hypothyroidism requiring only hormone replacement, vitiligo, psoriasis, or alopecia)
20. Requires systemic immunosuppressive treatment including, but not limited to (cyclophosphamide, azathioprine, methotrexate, thalidomide, and anti-tumor necrosis factor [anti-TNF] agents). Patients who have received or are receiving acute, low dose, systemic immunosuppressant medications (e.g., dexamethasone or prednisolone) may be enrolled. Replacement therapy (e.g., thyroxine, insulin, physiologic corticosteroid replacement therapy ((e.g., ≤10 mg/day of prednisone equivalents) for adrenal or pituitary insufficiency) is not considered a form of systemic treatment. The use of inhaled corticosteroids and mineralocorticoids (e.g., fludrocortisone), topical steroids, intra-nasal steroids, intra-articular, and ophthalmic steroids is allowed
21. Tumor-related pain (>grade 3) unresponsive to broad analgesic interventions (oral and/or patches).
22. Uncontrolled hypercalcemia, despite use of bisphosphonates
23. Any other diseases, metabolic dysfunction, physical examination finding, or clinical laboratory finding giving reasonable suspicion of a disease or condition that contraindicates the use of an investigational drug or that may affect the interpretation of the results or render the patient at high risk from treatment complications
24. Received an organ transplant(s)
25. Undergoing dialysis

Specific Additional Exclusion Criteria for (Hepato) Biliary Cancers (HCC for Part 1 and CCA for Part 1 and Part 2)

1. Any ablative therapy (Radio Frequency Ablation or Percutaneous Ethanol Injection) for HCC<6 weeks prior trial entry

2. Hepatic encephalopathy or severe liver adenoma

3. Child-Pugh score ≥7

4. Metastatic hepatocellular carcinoma that progressed while receiving at least one previous line of systemic therapy, including sorafenib, or who are intolerant of or refused sorafenib treatment following progression on standard therapy including surgical and/or local regional therapies, or standard therapy considered ineffective, intolerable, or inappropriate or for which no effective standard therapy is available

5. Biliary or gastric outlet obstruction allowed provided it is effectively drained by endoscopic, operative, or interventional means

6. Pancreatic, biliary, or enteric fistulae allowed provided they are controlled with an appropriate non-infected and patent drain (if any drains or stents are in situ, patency needs to be confirmed before the study start).

### Study Assessments

The schedule of assessments is divided into 2-week cycles after the pre-dose screening, which may take place up to 4 weeks prior to commencement of treatment. Study assessments include medical and physical examinations performed by a qualified physician, practitioner, or physician assistant. Medical history taken includes oncology history, radiation therapy history, surgical history, current and past medication. Assessments include restaging scan (CT with contrast, MRI with contrast, PET-CT (diagnostic CT) and/or X-ray).

Assessments also include Tumor biopsies (starting pre dose 1 and repeat biopsy as feasible)—depending upon scan(s). Alternatively, archival tissue may be used pre-dose.

Relevant tumor markers per tumor type—e.g., Ca15-3, CA-125, CEA, CA19-9, alpha fetoprotein, etc., are assessed every cycle pre-dose (which may be decreased to every 3 cycles after 6 months of treatment, following the same schedule as restaging scans), as appropriate. Assessments further include vital signs, ECOG, adverse events, blood count, blood chemistry, blood coagulation (prothrombin time (PT) and partial thromboplastin time (PTT), activated partial thromboplastin time (APTT)), blood and tumor biomarker analysis (immune phenotyping, cytokine measurement) and urine analysis (specific gravity, protein, white blood cell-esterase, glucose, ketones, urobilinogen, nitrite, WBC, RBC, and pH). Serum chemistry includes glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, HgbA1c, blood urea nitrogen, CPK, TSH, fT4, lipase, amylase, PTH, testosterone, estradiol. prolactin, FSH, LH, CRP.

CT with contrast is the preferred modality for restaging Scans—(MM, PET-CT and/or other imaging modalities instead of or in addition to the CT scan if CT is not feasible or appropriate, given location of the disease). Assessments are done every 6 to 8 weeks +/−1 week and at the End of Treatment if not assessed within the last 4 to 6 weeks.

Patients' blood samples are collected for routine clinical laboratory testing, and include hematology and serum chemistry.

Blood chemistry includes the following glucose, Hgb A1c, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, lipase, amylase, PTH, testosterone, estradiol. prolactin, FSH, LH, CRP.

### Investigational Product, Dose, and Administration

The anti-Gal9 antibody is administered via intravenous (IV) infusion every two weeks (Q2W) until progression of disease, unacceptable toxicity, or withdrawal of consent. Subjects who experience a dose-limiting toxicity may resume the anti-Gal9 antibody administration if the patient is experiencing benefit, after a discussion with the Study Medical Monitor. Dose reduction of up to 25% or as per the clinical discretion of the investigator and with agreement of the Medical Monitor and the Sponsor.
Stage 1: Subjects receive the anti-Gal9 antibody alone in accordance with the CRM design.
Stage 2: Subjects receive the RP2D of the anti-Gal9 antibody as a single agent or the antibody in combination with anti-PD-1 using the RP2D identified within Stage 1. The dose of the anti-Ga9 antibody is reduced (e.g., by 25% reduction) if a patient exhibits toxicity.
Stage 3: Treatment arms where efficacy is observed in Stage 2 is to be used in Stage 3 and expanded accordingly at dose levels tested in Stage 2, i.e., where the ORR/patient survival (depending on tumor type) is beyond the minimum threshold defined.

Other Drugs
Combination Drug: an approved Anti-PD-1 mAb (e.g., those noted above);
Doses: Anti-PD-1 mAb dose is determined depending on approved drug to be determined by initial IND.
Mode of Administration: Intravenous infusion

### Statistical Methods

Subject, disease and all clinical safety data are presented descriptively as means, medians, or proportions, with appropriate measures of variance (i.e., 95% CI, range). Waterfall and Swimmers plots is used to graphically present the RR and duration of responses for patients for each study arm, within each disease site. All efficacy analyses will be based on the ITT population unless otherwise specified. Survival curves for progression free survival (PFS) and overall survival (OS) is generated by the method of Kaplan-Meier. There is no comparative analysis between any of the six study arms. Exploratory correlations analysis is also undertaken to identify potential biomarkers and other predictors that are potentially associated with ORR, PFS and OS. All the statistical analyses will be performed using SAS, version 9.2. (SAS, Cary, N.C.).

### Study Procedures and Evaluations

Incidence and severity of AEs/CTAEs and SAEs, including clinically significant changes in laboratory parameters, vital signs & ECGs
Incidence of DLTs, PK and PD
Plasma PK parameters (e.g., AUC0-24 h, Gm., Tmax, estimated half-life); serum concentration vs. time profiles
Objective response rate (complete response and partial response) (ORR) and clinical benefit rate (objective response and stable disease 3 months or longer); progression free survival (PFS), overall survival (OS), disease control rate (DCR)
Blood and tumor immunophenotyping, galectin-9 serum, plasma levels, and tissue expression levels and expression pattern of tumor, stroma, immune cells), time to response (TTR), and other biomarker analysis, CT (PET) imaging, other clinically relevant imaging.

### Safety Monitoring

Routine safety monitoring is performed by the Medical Monitor. Safety monitoring, including analysis of PK, will be performed by a Safety Monitoring Committee (SMC), consisting of the Principal Investigators (and co-investigators as needed) and sponsor representatives and the study-specific Medical Monitor. Additional investigators and study team members will participate in reviews as needed. An Independent Data Monitoring Board is not be utilized for this open-label study.

In Stage 1, the dose-escalation phase, dose escalation to the next cohort proceeds following review of Cycle 1 of each cohort. Safety and available PK data are used to assess for a dose-limiting toxicity (DLT) in all patients of each cohort by the SMC. As a safety precaution, during dose escalation, new patients are entered and treated only after the first patient of each cohort has been treated with the anti-Gal9 antibody and at a minimum 7 days post-treatment has elapsed. Select DLT safety analysis for each patient is performed following completion of Cycle 1.

Dose-limiting toxicity (DLT) is defined as a clinically significant hematologic or non-hematologic adverse event or abnormal laboratory value assessed as unrelated to metastatic tumor disease progression, intercurrent illness, or concomitant medications and is related to the study drug and occurring during the first cycle on study that meets any of the following criteria:
All Grade 4 hematologic and non-hematologic toxicities of any duration
All Grade 3 hematologic and non-hematologic toxicities. Exceptions are as follow:
   Grade 3 nausea, vomiting and diarrhea that does not require hospitalization or TPN support and can be managed with supportive care to ≤grade 2 within 48 hours.
   Grade 3 electrolyte abnormalities that are corrected to ≤grade 2 within 24 hours.

Other grade 3 asymptomatic laboratory abnormalities DLT Period=One (1) cycle, i.e. two doses of G9.2-17 IgG4 on days 1 and 15 of each cycle.

Patients should ordinarily be maintained on study treatment until confirmed radiographic progression. If the patient has radiographic progression but no unequivocal clinical progression and alternate treatment is not initiated, the patient may continue on study treatment, at the investigator's discretion. However, if patients have unequivocal clinical progression without radiographic progression, study treatment is stopped and patients advised regarding available treatment options.

Both the approved checkpoint inhibitor and G9.2-17 IgG4 is withheld in the event of a serious or life-threatening immune related adverse reaction (IMAR) or one that prompts initiation of systemic steroids, although specific exceptions (e.g., for certain endocrinopathies in clinically stable patients) may be described in the approved product labeling.

If the protocol proposes continuation of an experimental agent in the setting of either (a) withholding the approved checkpoint inhibitor, or (b) initiation of systemic. steroids for an IMAR, provide sufficient justification supporting the safety of such an approach.

In the event where dose-reduction is used for AE management, two dose reductions are allowed. By 30% of the baseline dose at each dose reduction. Dose reductions are to be pursued only if the investigator assesses that clinical benefit is being and may continue to be derived.

Treatment emergent adverse events (TEAEs) will be defined as events that occur on or after the first dose of study medication. The Medical Dictionary for Regulatory Activities (MedDRA) coding dictionary will be used for the coding of AEs. TEAEs, serious or CTC grade 3 or 4 TEAEs, and TEAEs related to therapy will be summarized overall and by system organ class and preferred term by treatment group. These will summarize the number of events and the number and percent of patients with a given event. In addition, the number and percent of patients with TEAEs will be provided by maximum severity. A summary of all TEAEs by system organ class and preferred term occurring in at least 5 percent of patients in either treatment group will be provided.

Any AE≥Grade 3 possible, probably, or definitely related to one or more study drugs will be discussed with the Medical Monitor before continuing with dosing, with the following exceptions, for which no discussion with the Medical Monitor will be required:
Local injection site reactions lasting <72 hours including pain, redness, swelling, induration, or pruritus
Systemic injection reactions lasting <72 hours of fever, myalgia, headache, or fatigue

Where judged appropriate by the Investigator (after discussion with the Medical Monitor) a dose delay may be necessary for ≥Grade 3 adverse events until resolution of the toxicity (to Grade 1 or less).

In Part 2 of the protocol, if one or more patients develop a DLT, the dose of G9.2-17 IgG4 will be reduced to 1 dose below the recommended Phase 2 dose (RP2D).

Once a patient has completed the treatment period, overall survival follow-up is performed every 3 months for up to 2 years. Radiological assessment continues, where possible, for patients withdrawing due to clinical progression.

The following procedures will be done on Day 59 or thirty days after the last dose, including patients who have discontinued treatment early.
Restaging scan (CT with contrast, MM, PET-CT or X-ray)—repeat if end of study is >6 to 8 weeks after last cycle and in shorter intervals it investigator's discretion
Relevant tumor marker—e.g., Ca15-3, CA-125, CEA, CA19-9, alpha fetoprotein, etc., will be assessed every cycle pre-dose (which may be decreased to every 3 cycles after 6 months of treatment, following the same schedule as restaging scans), as appropriate
12-lead ECG
Physical examination
ECOG
Vital signs (temperature, HR, BP, RR, including weight) post-supine for 5 minutes
Concomitant medications (name, indication, dose, route, start and end dates)
Adverse events
Pregnancy test, if female
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, PTH, Estradiol. prolactin, testosterone, FSH, LH
Blood coagulation (PT, PTT)
Urinalysis
PD blood—biomarker analysis
PK blood samples

### RECIST Criteria for Tumor Assessment

At the baseline tumor assessment, tumor lesions/lymph nodes are categorized as measurable or non-measurable with measurable tumor lesions recorded according to the longest diameter in the plane of measurement (except for pathological lymph nodes, which are measured in the shortest axis). When more than one measurable lesion is present at baseline all lesions up to a maximum of five lesions total (and a maximum of two lesions per organ) representative of all involved organs should be identified as target lesions. Target lesions are selected on the basis of their size (lesions with the longest diameter). A sum of the diameters for all target lesions is calculated and reported as the baseline sum diameters.

All other lesions (or sites of disease) including pathological lymph nodes is identified as non-target lesions and are also be recorded at baseline. Measurements are not required and these lesions are followed as ‘present’, ‘absent’, or ‘unequivocal progression’.

Disease response (complete response (CR), partial response (PR), stable disease (SD), and progressive disease (PD)) is be assessed as outlined below.

The disease response measures allow for the calculation of the overall disease control rate (DCR), which includes CR, PR, and SD, the objective response rate (ORR), which includes CR and PR, progression-free survival (PFS), and time to progression (TTP).

### The Response Evaluation Criteria in Solid Tumors (RECIST) Guidelines

The overall response according to RECIST 1.1 is derived from time-point response assessments based on tumor burden as follows below.

Evaluation of Target Lesions:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

Evaluation of Non-Target Lesions:
Complete Response (CR): Disappearance of all non-target lesions and normalization of tumor marker level. All lymph nodes must be non-pathological in size (<10 mm short axis).
Non-CR/Non-PD: Persistence of one or more non-target lesion(s) and/or maintenance of tumor marker level above the normal limits.
Progressive Disease (PD): Unequivocal progression of existing non-target lesions. (Note: the appearance of one or more new lesions is also considered progression).

ECOG Performance Status*

| Grade | ECOG |
|---|---|
| 0 | Fully active, able to carry on all pre-disease performance without |
| | restriction |
| 1 | Restricted in physically strenuous activity but ambulatory and |
| | able to carry out work of a light or sedentary nature, e.g., light |
| | house work, office work |
| 2 | Ambulatory and capable of all self-care but unable to carry out |
| | any work activities. Up and about more than 50% of waking |
| | hours |
| 3 | Capable of only limited self-care, confined to bed or chair more |
| | than 50% of waking hours |
| 4 | Completely disabled. Cannot carry on any self-care. Totally |
| | confined to bed or chair |
| 5 | Dead |

As published in Am J Clin Oncol:
Oken MM, Creech RH, Tormey DC, et al. Toxicity and response criteria of the Eastern Cooperative Oncology Group. Am J Clin Oncol. 1982;5:649-655.

### Example 2: Anti-Galectin-9 Antibody Stability Study

The candidate IgG4 antibody underwent stability analysis after storage under several different conditions and at different concentrations. Stability analysis was performed via size exclusion chromatography (SEC) using a TOSOH TSKgel Super SW mAb column. SEC profiles before and after storage were compared to identify any issues with protein stability (e.g., aggregation or degradation).

### Materials and Methods

### Sample Preparation

The anti-Galectin-9 antibody was stored at −80° C. until use. Prior to analysis, samples were thawed in a room temperature water bath and stored on ice until analysis. Prior to handling, absorbance at 280 nm was measured using Nanodrop. The instrument was blanked using TBS (20 mM Tris pH 8.0, 150 mM NaCl). The sample was then transferred to polypropylene microcentrifuge tubes (USA Scientific, 1615-5500) and centrifuged at 4° C., 16.1k×g for 30 minutes. Samples were filtered through a 0.22 μm filter (Millipore; SLGV004SL). Post-filtration absorbance was measured.

### HPLC Analysis

Sample conditions tested included the following: ambient stability (0 hours at room temperature, 8 hours at room temperature), refrigerated stability (0 hours at 4° C., 8 hours at 4° C., 24 hours at 4° C.), and freeze/thaw stability (1× freeze/thaw, 3× freeze/thaw, 5× freeze/thaw). Each condition was run in duplicate at three different concentrations: stock, 10× dilution, and 100× dilution. One hundred μL samples were prepared for each condition and stored in a polypropylene microcentrifuge tube. Dilutions were prepared in TBS when necessary. Absorbance at 280 nm was read prior to analysis. Room temperature samples were stored on the benchtop for the durations indicated. 4° C. samples were either stored on ice or in 4° C. refrigerator for the periods indicated in Table 3. Freeze-thaw samples were snap-frozen in liquid nitrogen and then thawed in a room temperature water bath. The freeze and thaw process was performed either once, three or five times, and then the samples were stored at 4° C. until analysis.

SEC analysis was performed using a TOSOH TSKgel SuperSW mAb HR column on a Shimadzu HPLC with a UV detector at 280 nm. The columns were loaded with 25 μL of sample and run at 0.5 mL/min for 40 minutes. The KBI buffer formulation was used as the mobile phase.

### Results

The concentrations of the antibody were determined using UV absorbance measurements before and after filtration, as shown in Table 3. Two 2 mL samples supplied by KBI were thawed, one vial for use in room temperature and freeze/thaw conditions, and the other vial for use in the 4° C. conditions. Absorbance readings showed nearly complete recovery after filtration.

TABLE 3
Protein Recovery after Sample Preparation

| | | Pre-Filtration | Post- | Recovery |
|---|---|---|---|---|
| Vial | Read | (mg/mL) | Filtration (mg/mL) | (%) |
| 1 | 1 | 9.574 | 9.416 | 98.4 |
| (Used for | 2 | 9.435 | 9.553 | 101.3 |
| RT and | 3 | 9.504 | 9.541 | 100.4 |
| Freeze/ | Average | 9.50 ± 0.07 | 9.50 ± 0.07 | 100.0 ± 1.5 |
| Thaw) | | | | |
| 2 | 1 | 9.618 | 9.401 | 98.6 |
| (Used for | 2 | 9.814 | 9.704 | 98.9 |
| 4° C.) | 3 | 9.451 | 9.394 | 99.4 |
| | Average | 9.63 ± 0.18 | 9.53 ± 0.16 | 98.9 ± 0.4 |

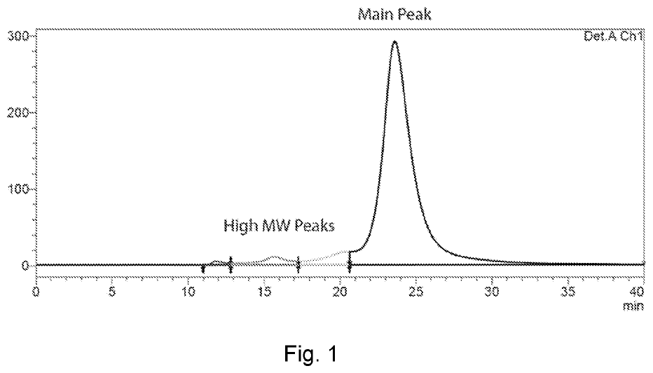

Two or three high molecular weight peaks that eluted earlier than the main peak were observed (FIG. 1). These peaks comprised approximately 5% of the total sample under each condition assayed (Table 4). No significant differences in protein concentration were observed under all assayed conditions.

TABLE 4
Stability Results

| | | Dilution | | Concentration | High Molecular Weight Peaks | | | | |
|---|---|---|---|---|---|---|---|---|---|
| Condition | Time | Sample | | (mg/mL) | 1 | 2 | 3 | Total | Main |
| | | 1 | 1 | 9.3 ± 0.3 | 0.06 | 3.024 | 4.307 | 7.39 | 92.61 |
| | | | 2 | 9.36 ± 0.03 | 0.615 | 0.273 | 3.822 | 4.71 | 95.29 |
| Room | 0 hr | 10 | 1 | 0.96 ± 0.012 | 0.34 | 1.18 | 3.183 | 4.70 | 95.30 |
| Temperature | | | 2 | 1.00 ± 0.02 | 0.418 | 1.225 | 2.541 | 4.18 | 95.82 |
| | | 100 | 1 | 0.147 ± 0.003 | 0.25 | 2.1278 | 2.472 | 4.85 | 95.15 |
| | | | 2 | 0.14 ± 0.05 | 0.17 | 1.507 | 2.684 | 4.36 | 95.64 |
| | 8 hr | 1 | 1 | 9.5 ± 0.19 | 0.597 | 1.41 | 1.997 | 4.00 | 96.00 |
| | | | 2 | 9.46 ± 0.04 | 0.501 | 1.219 | 2.147 | 3.87 | 96.13 |
| | | 10 | 1 | 1.03 ± 0.02 | 0.413 | 1.173 | 2.51 | 4.10 | 95.90 |
| | | | 2 | 1.026 ± 0.002 | 0.367 | 1.22 | 2.592 | 4.18 | 95.82 |
| | | 100 | 1 | 0.14 ± 0.012 | 0.839 | 1.584 | 2.342 | 4.77 | 95.24 |
| | | | 2 | 0.104 ± 0.008 | 0.723 | 1.578 | 2.719 | 5.02 | 94.98 |
| 4° C. | 1 hr | 1 | 1 | 9.68 ± 0.05 | 0.623 | 1.489 | 2.066 | 4.18 | 95.82 |
| | | | 2 | 9.6 ± 0.15 | 0.463 | 1.617 | 2.999 | 5.08 | 94.92 |
| | | 10 | 1 | 0.96 ± 0.03 | 0.436 | 1.122 | 2.438 | 4.00 | 96.00 |
| | | | 2 | 0.96 ± 0.02 | 0.432 | 1.173 | 2.799 | 4.40 | 95.60 |
| | | 100 | 1 | 0.106 ± 0.003 | 0.503 | 1.834 | 2.73 | 5.07 | 94.93 |
| | | | 2 | 0.103 ± 0.004 | 0.538 | 1.603 | 2.789 | 4.93 | 95.07 |
| | 8 hr | 1 | 1 | 9.59 ± 0.07 | 0.285 | 1.135 | 2.699 | 4.12 | 95.88 |
| | | | 2 | 9.87 ± 0.010 | 0.382 | 0.85 | 2.74 | 3.97 | 96.03 |
| | | 10 | 1 | 0.99 ± 0.015 | 1.342 | 1.168 | 2.647 | 5.16 | 94.84 |
| | | | 2 | 0.98 ± 0.03 | 0.901 | 1.79 | 2.547 | 5.24 | 94.76 |
| | | 100 | 1 | 0.100 ± 0.002 | 0 | 1.768 | 4.856 | 6.62 | 93.38 |
| | | | 2 | 0.097 ± 0.003 | 0 | 0.98 | 3.653 | 4.63 | 95.37 |
| | 24 hr | 1 | 1 | 9.60 ± 0.04 | 0.466 | 1.563 | 2.988 | 5.02 | 94.98 |
| | | | 2 | 9.68 ± 0.08 | 0.491 | 1.166 | 2.521 | 4.18 | 95.82 |
| | | 10 | 1 | 0.973 ± 0.005 | 0.579 | 1.095 | 2.888 | 4.56 | 95.44 |
| | | | 2 | 0.98 ± 0.04 | 0.36 | 1.106 | 2.488 | 3.95 | 96.05 |
| | | 100 | 1 | 0.097 ± 0.001 | 0.588 | 1.413 | 2.95 | 4.95 | 95.05 |
| | | | 2 | 0.099 ± 0.002 | 0.587 | 1.463 | 2.886 | 4.94 | 95.06 |
| Freeze | 1× | 11 | 1 | 9.5 ± 0.10 | 0.439 | 1.143 | 2.292 | 3.87 | 96.13 |
| Thaw | | | 2 | 9.04 ± 0.08 | 0.489 | 1.597 | 2.58 | 4.67 | 95.33 |
| | | 10 | 1 | 1.09 ± 0.03 | 0.388 | 1.228 | 2.741 | 4.36 | 95.64 |
| | | | 2 | 1.08 ± 0.05 | 0.387 | 1.243 | 2.932 | 4.56 | 95.44 |
| | | 100 | 1 | 0.12 ± 0.010 | 0.467 | 1.207 | 2.355 | 4.03 | 95.97 |
| | | | 2 | 0.11 ± 0.011 | 0.627 | 1.65 | 3.09 | 5.37 | 94.63 |
| | 3× | 1 | 1 | 8.1 ± 0.8 | 0.478 | 1.152 | 1.791 | 3.42 | 96.58 |
| | | | 2 | 9.0 ± 0.7 | 0.5 | 1.18 | 1.99 | 3.67 | 96.33 |
| | 5× | 1 | 1 | 8.9 ± 0.6 | 0.505 | 1.578 | 2.612 | 4.70 | 95.31 |
| | | | 2 | 8.6 ± 0.4 | 0.464 | 1.662 | 3.008 | 5.13 | 94.87 |

In summary, the anti-Galectin-9 antibody showed consistent stability after storage under all conditions analyzed, as indicated by no significant change in the SEC profile. There was no significant loss of protein after filtration, and two to three high molecular weight peaks were identified, comprising approximately 5% of the total sample. The results suggest that the antibody is stable under all conditions tested, with no aggregate formation or degradation observed.

### Example 3. Assessment of Galectin-9 Expression in Tumor Biopsy-Derived Organoid Fractions

Tumor organoids can be applied for the prediction of patient outcome, since the use of tumor models with similar characteristics to the original tumors may result in more accurate predictions of drug responses in patients. (See, e.g., Trends in Biotechnology; VOLUME 36, ISSUE 4, P358-371, Apr. 1, 2018).

Galectin-9 levels in a tumor may function as an indicator to predict a drug response. Biopsy derived organoids can be used as a proxy to assess levels of Galectin-9 in the original tumor. Accordingly, the ability to assess Galectin-9 levels in single cell or organoid fractions was tested.

Biopsies were received from representative pancreatic adenocarcinoma and colorectal cancers and processed as follows. Human surgically resected tumor specimens were received fresh in DMEM media on ice and minced in 10 cm dishes. Minced tumors were resuspended in DMEM+10% FBS with 100 U/mL collagenase type IV to obtain spheroids. Partially digested samples were pelleted and then re-suspended in fresh DMEM+10% FBS and strained over both 100 mm and 40 mm filters to generate 51 (>100 mm), S2 (40-100 mm), and S3 (<40 mm) spheroid fractions, which were subsequently maintained in ultra-low-attachment tissue culture plates.

S2 fractions were digested by trypsin for 15 minutes to generate into single cells. For flow cytometry preparation, cell pellets from S2 and S3 fractions were re-suspended and cell labeling was performed after Fc receptor blocking (#422301; BioLegend, San Diego, Calif.) by incubating cells with fluorescently conjugated mAbs directed against human CD45 (HI30), CD3 (UCHT1), CD11b (M1/70), Epcam (9C4) and Gal9 (9M1-3; all Biolegend) or Gal9 Fab of G9.2-17 or Fab isotype. Dead cells were excluded from analysis using zombie yellow (BioLegend). Flow cytometry was carried out on the Attune NxT flow cytometer (Thermo Scientific). Data were analyzed using FlowJo v.10.1 (Treestar, Ashland, Oreg.).

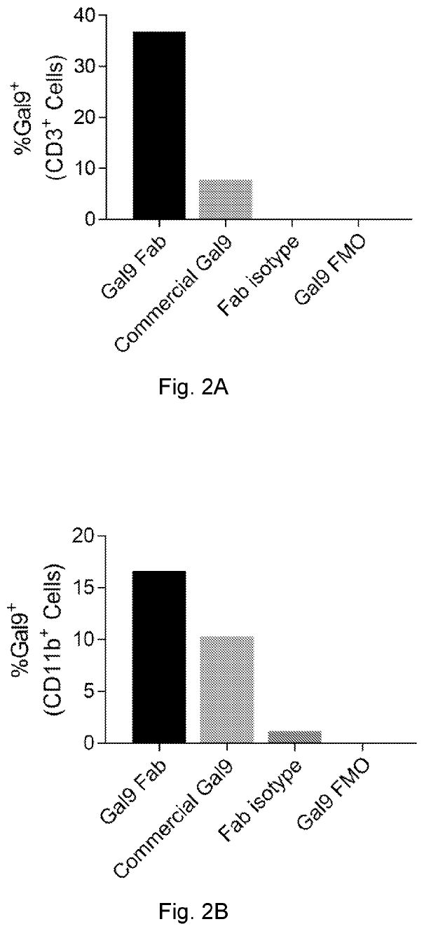

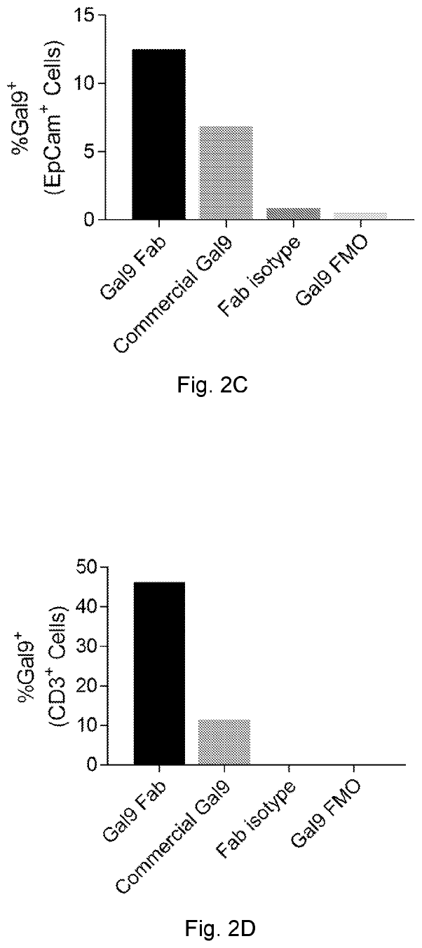

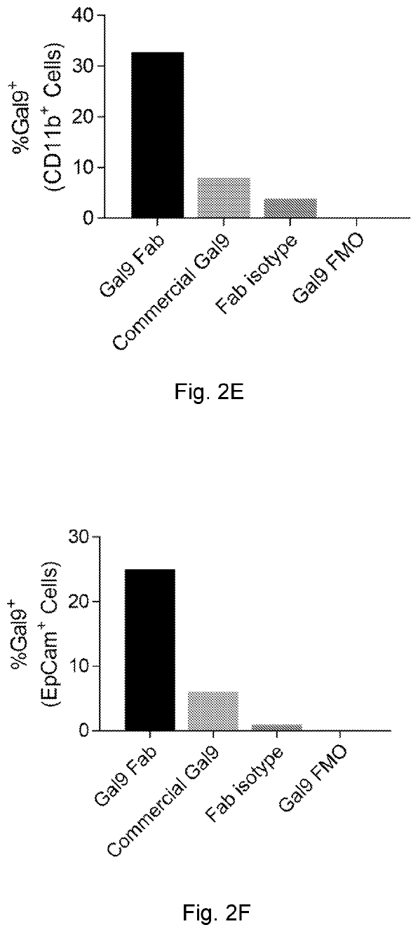

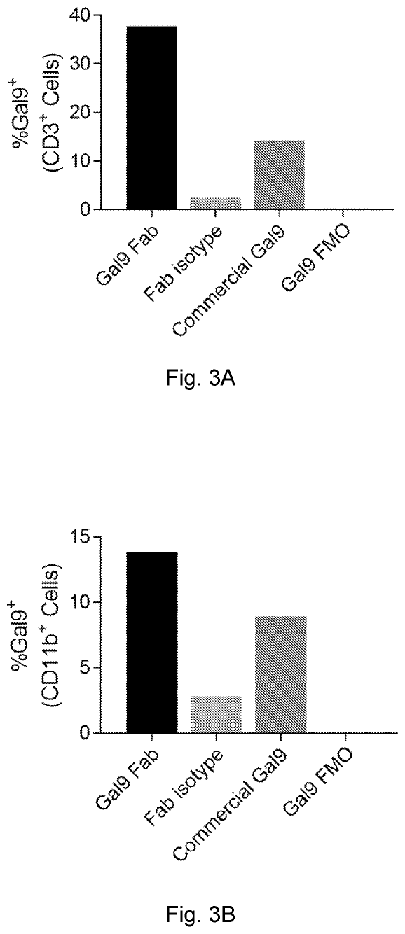

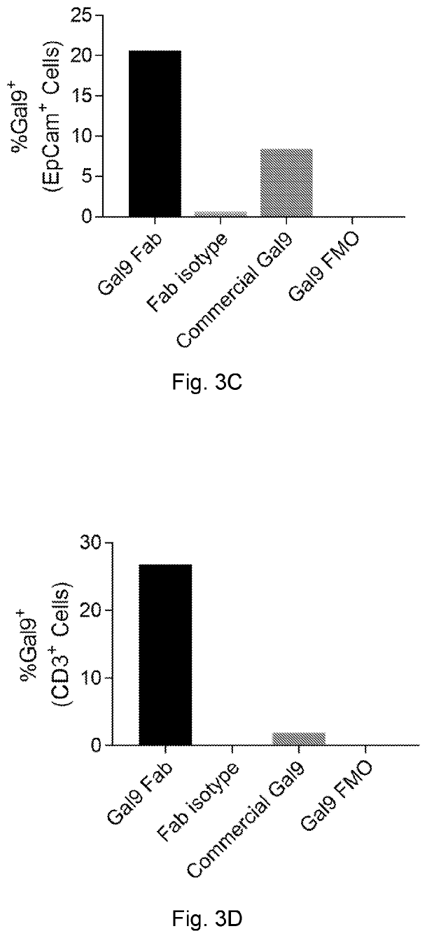

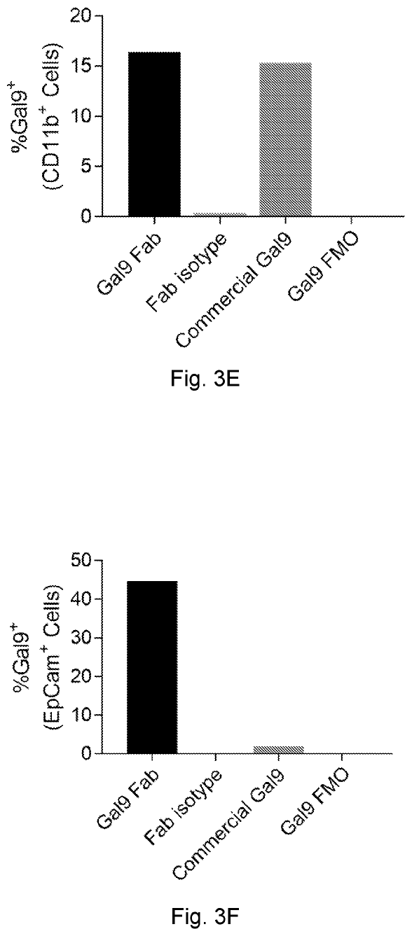

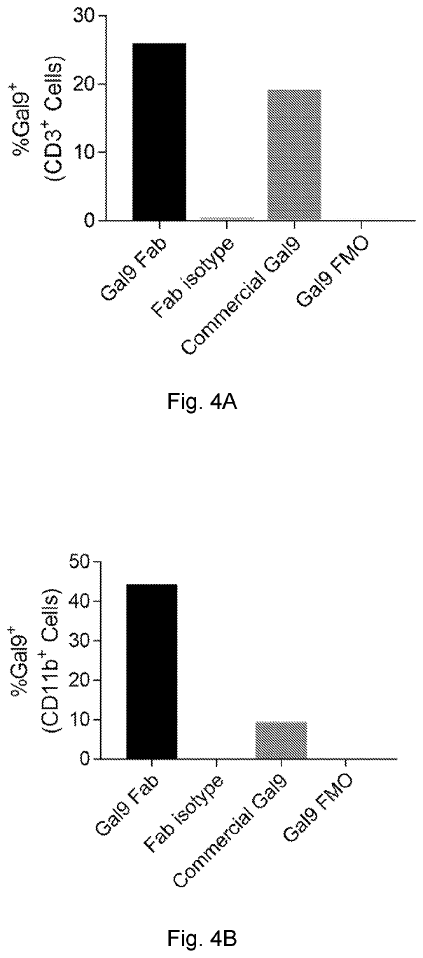

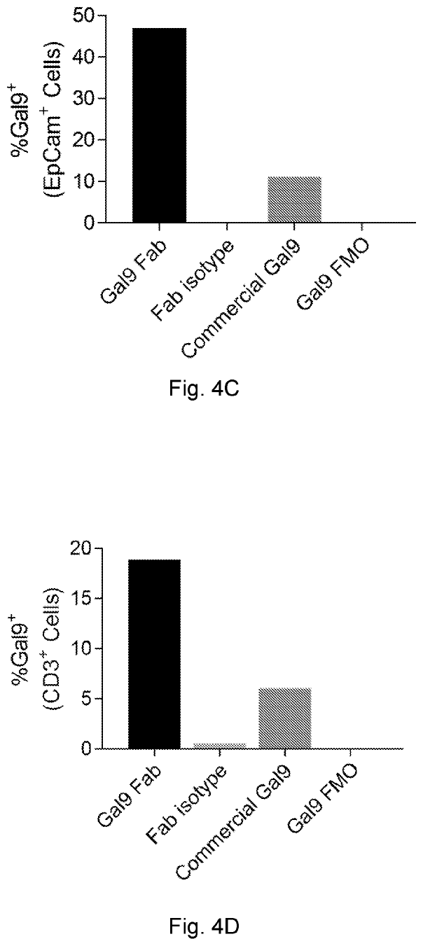

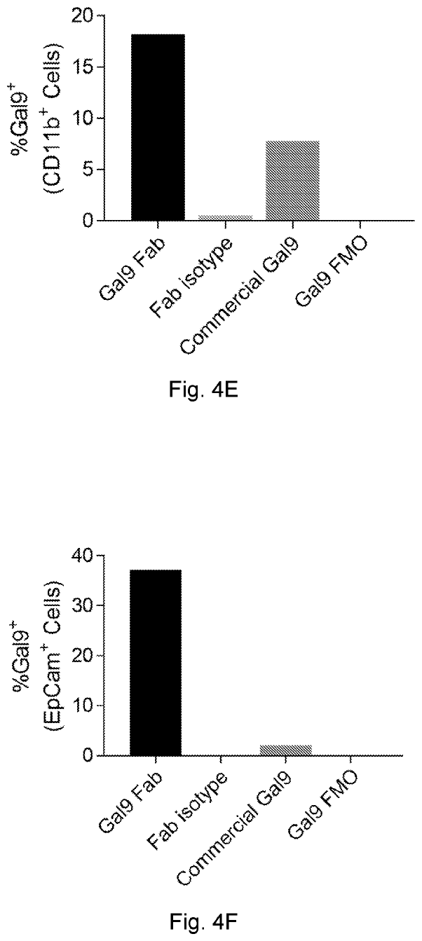

Results are shown in FIGS. 2A-2F, 3A-3F and 4A-4F and indicate that levels of Galectin-9 detected by the Gal9 G9.2-17 Fab in S2 single cell and S3 organoid fractions correlate. Accordingly, both S2 single cells and S3 organoids can be used for assessment of Galectin-9 levels in organoids derived from tumor biopsies.

### Example 4. Preparation of Patient-Derived Organotypic Tumor Spheroids (PDOTs) for Cellular Analysis

Biopsy-derived organoids can be a useful measure to assess the ability of a therapeutic to stimulate an immune response. Accordingly, S2 fractions described in the previous Example 3 above used for ex vivo culture were treated with anti-Galectin-9 antibody G9.2-17 and prepared for immune profiling.

An aliquot of the S2 fraction was pelleted and resuspended in type I rat tail collagen (Corning) at a concentration of 2.5 mg/mL following the addition of 10×PBS with phenol red with pH adjusted using NaOH. pH 7.0-7.5 was confirmed using PANPEHA Whatman paper (Sigma-Aldrich). The spheroid-collagen mixture was then injected into the center gel region of a 3-D microfluidic culture device as described in Jenkins et al., Cancer Discov. 2018 February; 8(2):196-215; Ex Vivo Profiling of PD-1 Blockade Using Organotypic Tumor Spheroids, the contents of which is herein incorporated by reference in its entirety. Collagen hydrogels containing patient-derived organotypic tumor spheroids (PDOTS) were hydrated with media with or without anti-Galectin-9 monoclonal antibody G9.2-17 after 30 minutes at 37° C. The PDOTS were then incubated at 37° C. for 3 days.

Cell pellets were re-suspended in the FACS buffer and 1×10⁶ cells were first stained with zombie yellow (BioLegend) to exclude dead cells. After viability staining, cells were incubated with an anti-CD16/CD32 mAb (eBiosciences, San Diego, Calif.) for blocking FcγRIII/II followed by antibody staining with 1 μg of fluorescently conjugated extracellular mAbs. Intracellular staining for cytokines and transcription factors was performed using the Fixation/Permeabilization Solution Kit (eBiosciences). Useful human flow cytometry antibodies included CD45 (HI30), CD3 (UCHT1), CD4 (A161A1), CD8 (HIT8a), CD44 (BJ18), TNFα (MAb11), IFNγ (4S.B3), and Epcam (9C4); all Biolegend. Flow cytometry was carried out on the LSR-II flow cytometer (BD Biosciences). Data were analyzed using FlowJo v.10.1 (Treestar, Ashland, Oreg.).

### Example 5. Assessment of Galectin-9 Levels in Plasma and Serum of Cancer Patients

Plasma and serum Galectin-9 levels were assessed in patient samples and compared to healthy volunteers. Blood (10 ml) was drawn from peripheral venous access from 10 healthy controls and 10 inoperable cancer patients. Serum and plasma were extracted from each sample of blood. Blood was collected in standard EDTA tubes PicoKine™ ELISA; Catalog number: EK1113 was used essentially according to manufacturer's instructions. Results of individual values are tabulated in Table 5 and Table 6.

TABLE 5
Patient Samples

| | | Serum | Plasma |
|---|---|---|---|
| Cancer Type | Patient No. | (pg/ml) | (pg/ml) |
| Breast cancer with metastases in liver | Patient 1 | 11362.29 | 12107.56 |
| and bones | | | |
| Melanoma brain and lung metastases | Patient 2 | 978.97 | 1106.79 |
| braf + | | | |
| Melanoma lung metastases braf − | Patient 3 | 838.83 | 695.08 |
| Rectal cancer with liver metastases | Patient 4 | 579.42 | 725.62 |
| Locally advanced gastric cancer | Patient 5 | 666.67 | 645.2 |
| Gastric cancer with liver, spleen and | Patient 6 | 674.3 | 877.69 |
| adrenal metastases | | | |
| Stage III ovarian cancer | Patient 7 | 1439.61 | 1341.6 |
| Metaststic canvcer of the unknown | Patient 8 | 1432.39 | 1671.8 |
| primary | | | |
| Testicular cancer | Patient 9 | 1352.56 | 1696.11 |
| Sarcoma | Patient 10 | 968.18 | 1073.57 |
| | Average | 2029.322 | 2194.102 |

TABLE 6
Healthy Volunteer Samples

| Sample Number | Serum | Plasma |
|---|---|---|
| Control 1 | 536.4 | 611.97 |
| Control 2 | 476.43 | 592.58 |
| Control 3 | 612.66 | 651.43 |
| Control 4 | 269.75 | 414.41 |
| Control 5 | 460.26 | 602.28 |
| Control 6 | 206.66 | 405.8 |
| Control 7 | 385.88 | 439.85 |
| Control 8 | 525.283 | 654.2 |
| Control 9 | 711.047 | 718.68 |
| Control 10 | 296.85 | 349.09 |
| Average | 448.122 | 544.029 |

### Example 6. Assessment of Galectin-9 Expression and Localization Using Immunohistochemical Analysis

The ability to use immunohistochemical analysis to determine Galectin-9 expression levels in tumors was assessed using paraffin-embedded biopsy-derived tumor samples.

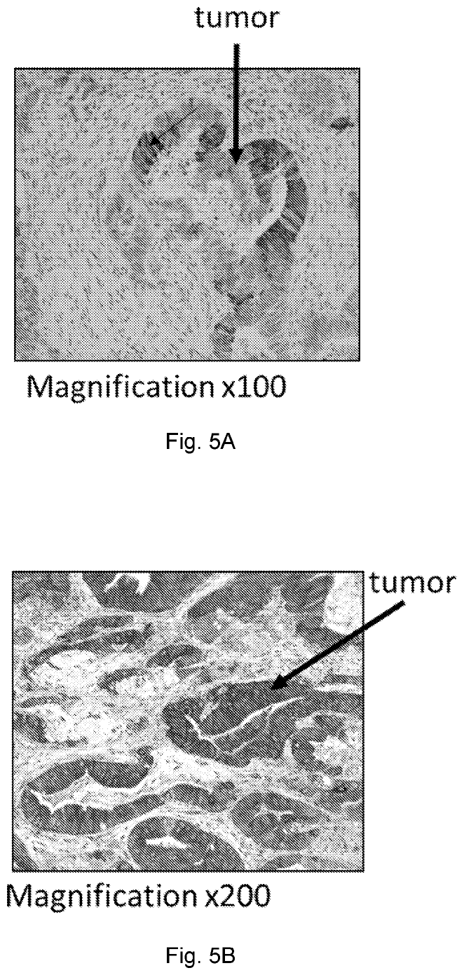

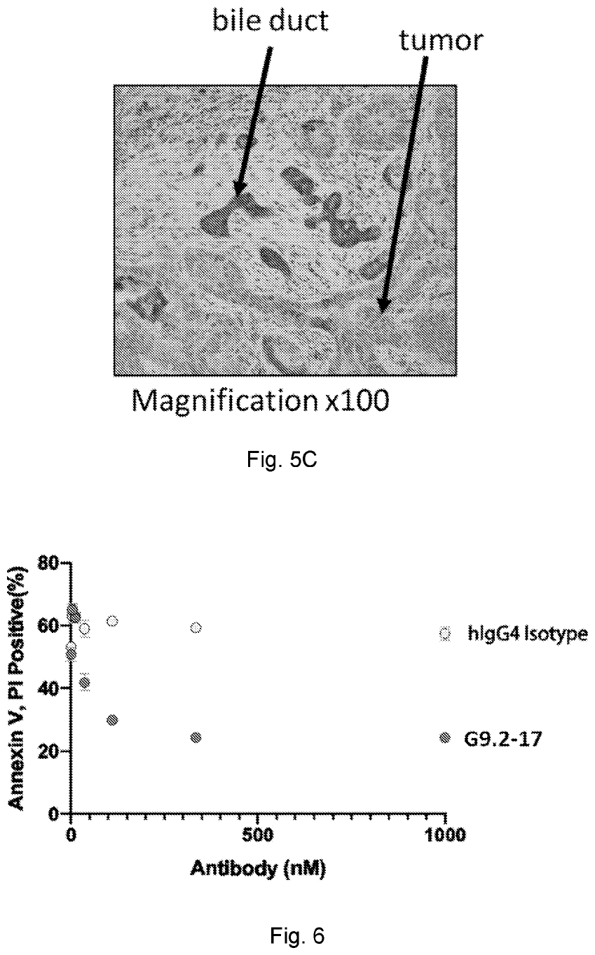

In brief, slides were deparaffinized (xylene: 2× 3 min; absolute alcohol: 2×3 min., methanol: 1×3 min) and rinsed in cold tap water. For antigen retrieval, citrate buffer (pH 6) was preheated to 100° C. in a water bath and slides were incubated in citrate buffer for 5 minutes. Slides were left to cool for about 10 min at room temperature and put in running water. Slides were washed in PBS, a pap pen circle was drawn around the section, and sections were incubated in blocking buffer (DAKO-Peroxidase blocking solution-52023) for 5 minutes. Serum free blocker was added (Novocastra serum free Protein Blocker), and then rinsed off with PBS. Primary antibody (Sigma, anti-Galectin-9 clone 1G3) was used at 1:2000 dilution in DAKO-52022 diluent and sections were incubated over night at 4° C. Slides were washed with PBS and then incubated with the secondary antibody (anti-mouse) for 45 minutes at room temperature. Slides were washed and stained with ABC VECTOR STAIN (45 mins), washed with PBS, stained with DAB (1 ml stable DAB buffer+1 drop DAB)) for 5 minutes and washed in running water. Haematoxylin was added for 1 minute and 70% ETOH+1% HCL was applied to avoid over staining. Slides were left in running water for 2-3 min, then dipped in water, then absolute alcohol, and then xylene, 2 times for 30 seconds each. Cover slip and images were captured. Galectin-9 staining in a chemotherapy treated colorectal cancer and a liver metastasis of colorectal carcinoma are shown in FIGS. 5A and 5B. Results from Galectin-9 negative cholangiocarcinoma is shown in FIG. 5C.

### Example 7. Cross-Reactivity of Anti-Galectin-9 Antibody G9.2-17 with Other Galectins

In order to assess antibody specificity and cross-reactivity with other Galectins, anti-Galectin-9 antibody G9.2-17 was tested for binding against a human proteomic array consisting all members of the Galectin family—and at two working concentrations. Antibody specificity was evaluated using CDI's HuProt Human Proteome Microarray (˜75% of the human proteome). The microarray was incubated with the primary antibody, rinsed, incubated with a fluorescently-labelled secondary antibody and subsequently analyzed for the amount of fluorescence detected for each target protein. Results were compiled as microarray images. The results indicated that anti-Galectin-9 antibody G9.2-17 is highly specific to Galectin-9 and does not cross-react with any other Galectin family members.

### Example 7. Anti-Galectin-9 Antibody Protects T Cells from Galectin-9 Mediated Apoptosis

To investigate actions of anti-Galectin-9 antibody G9.2-17, an apoptosis assay was performed to determine if T cells are dying by the process of apoptosis or by other mechanisms.

In brief, MOLM-13 (human leukemia) cells were cultured in RPMI media supplemented with 10% FBS, 2 mM L-glutamine, 10 mM HEPES, 1 mM sodium pyruvate, 4.5 g/L glucose and 1.5 g/L sodium bicarbonate at 37° C. in 5% CO₂. Cells were then transferred into serum-free RPMI media and suspended at a concentration of 2.5e6 cells/mL in serum-free media. Cells were seeded into the wells of a tissue culture grade 96-well plate at a density of 2e5 cells/well (80 μL of cell suspension per well). Monoclonal anti-Galectin-9 antibody or matched isotype was added to each well and incubated at 37° C., 5% CO₂ for 30 min. Following this incubation, recombinant, full length human Galectin-9 (R&D Systems 2045-GA, diluted in PBS) was added to a final concentration of 200 nM. Cells were incubated at 37° C., 5% CO₂ for 16 hours. Cells were then stained with Annexin V-488 and propidium iodide (PI) prior to analysis by flow cytometry. Each condition was performed in triplicate. PI is impermeant to live cells and apoptotic cells, but stains dead cells with red fluorescence, binding tightly to the nucleic acids in the cell. After staining a cell population with Alexa Fluor® 488 annexin V and PI in buffer, apoptotic cells showed green fluorescence, dead cells showed red and green fluorescence, and live cells showed little or no fluorescence. The cells were distinguished using a flow cytometer with the 488 nm line of an argon-ion laser for excitation. Analysis was then performed on FlowJo software. The fraction of annexin V- and propidium iodide (PI)-positive cells is plotted as a function of antibody concentration used in FIG. 6. As shown in FIG. 6, the level of apoptotic T cells treated with the anti-Gal9 antibody was much lower than T cells treated with a human IgG4 isotype control antibody, indicating that the anti-Galectin-9 antibody G9.2-17 protects T cells against galectin-9 mediated cell apoptosis.

### Example 8: Evaluation of Gal-9 Antibodies Alone or in Combination with Checkpoint Inhibition in a Mouse Model of Pancreatic Cancer and Tumor Mass and Immune Profile of Mice Treated with G9.2-17 mIgG1

The effect of G9.2-17 mIgG1 on tumor weight and on immune profile was assessed in a mouse model of pancreatic cancer. 8-week old C57BL/6 male (Jackson Laboratory, Bar Harbor, Me.) mice were administered intra-pancreatic injections of FC1242 PDA cells derived from Pdx1Cre; KrasG12D; Trp53R172H (KPC) mice (Zambirinis C P, et al., TLR9 ligation in pancreatic stellate cells promotes tumorigenesis. J Exp Med. 2015; 212:2077-94). Tumor cells were suspended in PBS with 50% Matrigel (BD Biosciences, Franklin Lakes, N.J.) and 1×10⁵ tumor cells were injected into the body of the pancreas via laparotomy. Mice (n=10/group) received one pre-treatment dose i.p. followed by 3 doses (q.w.) of commercial αGalectin 9 mAb (RG9-1, 200 ug, BioXcell, Lebanon, N.H.) or G9.2-17 mIgG1 (200 μg), or paired isotype, either G9.2-Iso or rat IgG2a (LTF-2, BioXcell, Lebanon, N.H.) (200 m) (one dose per week for three weeks). Mice were sacrificed 3 weeks later and tumors were harvested for analyses by flow cytometry. Tissue was processed and prepared and flow cytometric analysis was performed following routine practice. See, e.g., U.S. Pat. No. 10,450,374.

Tumor Mass and Immune Profile of Mice Treated with G9.2-17 mIgG2a Alone or in Combination with αPD1 mAb

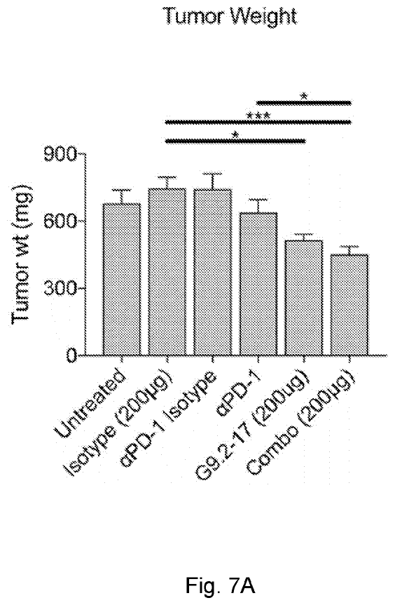

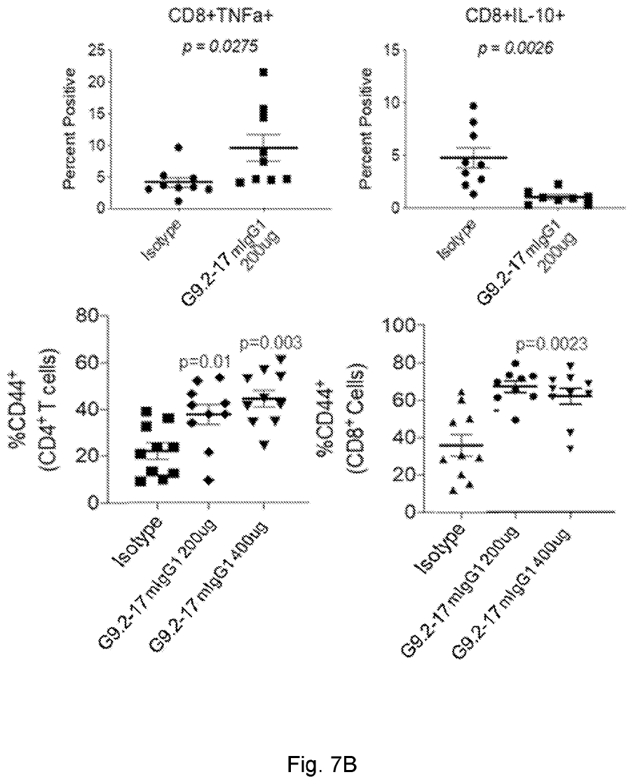

The effect of G9.2-17 mIgG2a on tumor weight and on immune profile was assessed in a mouse model of pancreatic cancer, alone or in combination with immunotherapy. 8-week old C57BL/6 male mice (Jackson Laboratory, Bar Harbor, Me.) were administered intra-pancreatic injections of FC1242 PDA cells derived from Pdx1Cre; KrasG12D; Trp53R172H (KPC) mice. Tumor cells were suspended in PBS with 50% Matrigel (BD Biosciences, Franklin Lakes, N.J.) and 1×105 tumor cells were injected into the body of the pancreas via laparotomy. Mice received one pre-treatment dose i.p. followed by 3 doses (q.w.) of G9.2-17 mIgG2a (200 μg) or a neutralizing αPD-1 mAb (29° F.1A12, 200 μg, BioXcell, Lebanon, N.H.), separately or in combination, or paired isotype (LTF-2 and C1.18.4, BioXcell, Lebanon, N.H.) as indicated. Mice were sacrificed on day 26 and tumors were harvested for analyses. Tissue was processed and prepared and flow cytometric analysis was performed following routine practice. See, e.g., U.S. Pat. No. 10,450,374. Each point represents one mouse; *p<0.05; **p<0.01; ***p<0.001; ****p<0.0001; by unpaired Student's t-test. These results show single-agent treatment with G9.2-17 mIgG2a reduces tumor growth at both of the dose levels, whereas anti-PD-1 alone had no effect on tumor size. FIG. 7.

### Example 9: Evaluation of Anti-Gal-9 Antibodies in Two Syngeneic Models of Colorectal and Melanoma Cancer in Immunocompetent Mice

Gal-9 antibodies G9.2-17 and G9.1-8 m13 are evaluated in syngeneic models of colorectal and melanoma cancer in immunocompetent mice. Structures of these two antibodies are either provided herein or disclosed in PCT/US2020/024767, the relevant disclosures of which are incorporated by reference for the subject matter and purpose referenced herein. Test articles are formulated and prepared on a weekly basis for the duration of the study.

### Experimental Design

Pre-study animals (female C57BL/6, 6-8 weeks of age (Charles River Labs) are acclimatized for 3 days and then are unilaterally implanted subcutaneously on the left flank with 5e5 B16.F10 (melanoma cell line) or MC38 cells (colorectal cancer cell line) resuspended in 100 μl PBS. Pre-study tumor volumes are recorded for each experiment beginning 2-3 days after implantation. When tumors reach an average tumor volume of 50-100 m^{m3} (preferably 50-75 mm³) animals are matched by tumor volume into treatment or control groups to be used for dosing and dosing initiated on Day 0. The study design for testing of Anti-Gal9 IgG1 and Anti-Gal9 IgG2 is summarized in Table 7 and Table 8.

TABLE 7
Anti-Gal9 IgG1 (B16F10 and MC38)

| | | | | | Route of | | Total |
|---|---|---|---|---|---|---|---|
| | | | Dose | Dose | Administration | | Number |
| Group | -n- | Test Agent | (μg/mouse) | Volume | (ROA) | Schedule | of Doses |
| 1 | 8 | Control Untreated | — | — | — | — | — |
| 2 | 8 | Control mIgG1 | 200 μg | 200 μl | IV | Q4Dx6 | 6 |
| 3 | 8 | Control mIgG1 | 400 μg | 200 μl | IV | Q4Dx6 | 6 |
| 4 | 8 | Control mIgG2 | 200 μg | 200 μl | IP | BIWx4 | 8 |
| 5 | 8 | Anti-Gal9 mIgG1 | 200 μg | 200 μl | IV | Q4Dx6 | 6 |
| 6 | 8 | Anti-Gal9 mIgG1 | 400 μg | 200 μl | IV | Q4Dx6 | 6 |
| 7 | 8 | Anti-Gal9 mIgG1 | 200 μg | 200 μl | IV | Q4Dx6 | 6 |
| | | (G9.1-8m13) | | | | | |
| 8 | 8 | Anti-Gal9 mIgG1 | 400 μg | 200 μl | IV | Q4Dx6 | 6 |
| | | (G9.1-8m13) | | | | | |
| 9 | 8 | Anti-Gal9 mIgG1 + | 200 μg | 200 μl | IV IP | Q4Dx6 | 68 |
| | | mAnti-PD 1 | 200 μg | 200 μl | | BIWx4 | |
| 10 | 8 | Anti-Gal9 mIgG1 + | 400 μg | 200 μl | IV IP | Q4Dx6 | 68 |
| | | mAnti-PD 1 | 200 μg | 200 μl | | BIWx4 | |
| 11 | 8 | Anti-Gal9 mIgG1 | 200 μg | 200 μl | IV IP | Q4Dx6 | 68 |
| | | (G9.1-8m13) +30 | 200 μg | 200 μl | | BIWx4 | |
| | | mAnti-PD 1 | | | | | |
| 12 | 8 | Anti-Gal9 mIgG1 | 400 μg | 200 μl | IV IP | Q4Dx6 | 68 |
| | | (G9.1-8m13) + | 200 μg | 200 μl | | BIWx4 | |
| | | mAnti-PD 1 | | | | | |
| 13 | 8 | mAnti-PD 1 | 200 μg | 200 μl | IP | BIWx4 | 8 |

TABLE 8
Anti-Gal9 IgG2 (B16F10 and MC38)

| | | | | | Route of | | Total |
|---|---|---|---|---|---|---|---|
| | | | Dose | Dose | Administration | | Number |
| Group | -n- | Test Agent | (μg/mouse) | Volume | (ROA) | Schedule | of Doses |
| 1 | 10 | Control Untreated | — | — | — | — | — |
| 2 | 10 | Control mIgG2 | 200 μg | 200 μl | IV | Q4Dx6 | 6 |
| 3 | 10 | Control mIgG2 | 400 μg | 200 μl | IV | Q4Dx6 | 6 |
| 4 | 10 | Control mIgG2 | 200 μg | 200 μl | IP | BIWx4 | 8 |
| 5 | 10 | Anti-Gal9 mIgG2 | 200 μg | 200 μl | IV | Q4Dx6 | 6 |
| 6 | 10 | Anti-Gal9 mIgG2 | 400 μg | 200 μl | IV | Q4Dx6 | 6 |
| 5 | 10 | Anti-Gal9 mIgG2 | 200 μg | 200 μl | IV | Q4Dx6 | 6 |
| | | (G9.1-8m13) | | | | | |
| 6 | 10 | Anti-Gal9 mIgG2 | 400 μg | 200 μl | IV | Q4Dx6 | 6 |
| | | (G9.1-8m13) | | | | | |
| 7 | 10 | Anti-Gal9 mIgG2 + | 200 μg | 200 μl | IV | Q4Dx6 | 68 |
| | | mAnti-PD 1 | 200 μg | 200 μl | IP | BIWx4 | |
| 8 | 10 | Anti-Gal9 mIgG2 + | 400 μg | 200 μl | IV IP | Q4Dx6 | 68 |
| | | mAnti-PD 1 | 200 μg | 200 μl | | BIWx4 | |
| 7 | 10 | Anti-Gal9 mIgG2 | 200 μg | 200 μl | IV | Q4Dx6 | 68 |
| | | (G9.1-8m13) + | 200 μg | 200 μl | IP | BIWx4 | |
| | | mAnti-PD 1 | | | | | |
| 8 | 10 | Anti-Gal9 mIgG2 | 400 μg | 200 μl | IV IP | Q4Dx6 | 68 |
| | | (G9.1-8m13) + | 200 μg | 200 μl | | BIWx4 | |
| | | mAnti-PD 1 | | | | | |
| 9 | 10 | mAnti-PD 1 | 200 μg | 200 μl | IP | BIWx4 | 8 |

Tumor volumes are taken three times weekly. A final tumor volume is taken on the day the study reaches endpoint. A final tumor volume is taken if an animal is found moribund. Animals are weighed three times weekly. A final weight is taken on the day the study reaches end point or if animal is found moribund. Animals exhibiting ≥10% weight loss when compared to Day 0 are provided DietGel® ad libitum. Any animal exhibiting >20% net weight loss for a period lasting 7 days or if mice display >30% net weight loss when compared to Day 0 is considered moribund and is euthanized. The study endpoint is set when the mean tumor volume of the control group (uncensored) reaches 1500 mm3. If this occurs before Day 28, treatment groups and individual mice are dosed and measured up to Day 28. If the mean tumor volume of the control group (uncensored) does not reach 1500 mm3 by Day 28, then the endpoint for all animals is the day when the mean tumor volume of the control group (uncensored) reaches 1500 mm3 up to a maximum of Day 60. Blood is collected from all animals from each group. For blood collection, as much blood as possible is collected via a cardiac puncture into K₂EDTA tubes (400 μl) and serum separator tubes (remaining) under deep anesthesia induced by isoflurane inhalation. The blood collected into K₂EDTA tubes is placed on wet ice until used for performing immune panel flow.

Blood collected into serum separator tubes is allowed to clot at room temperature for at least 15 minutes. Samples are centrifuged at 3500 for 10 minutes at room temperature. The resultant serum is separated, transferred to uniquely labeled clear polypropylene tubes, and frozen immediately over dry ice or in a freezer set to maintain −80° C. until shipment for the bridging ADA assay (shipped within one week).

Tumors from all animals are collected as follows. Tumors less than 400 mm³ in size are snap frozen, placed on dry ice, and stored at −80° C. until used for RT-qPCR analysis. For tumors of 400-500 mm³ in size, whole tumors are collected into MACS media for use in the Flow Panel. For tumors greater than 500 mm³ in size, a small piece (about 50 mm³) is snap frozen placed on dry ice, and stored at −80° C. for RT-qPCR, and the remaining tumor is collected in MACS media for flow cytometry. For flow cytometry, tumors are placed in MACS media and stored on wet ice until processed.

Spleen, liver, colon, lungs, heart, and kidneys from all animals are retained in 10% neutral buffered formalin (NBF) for 18-24 hours, transferred to 70% ethanol and stored at room temperature. Formalin fixed samples are paraffin embedded.

### Example 10: Evaluation of Gal-9 Antibody in a Models of Cholangiocarcinoma

The efficacy of Gal-9 antibody is assessed in a mouse model of cholangiocarcinoma as described in S. Rizvi, et al. (YAP-associated chromosomal instability and cholangiocarcinoma in mice, Oncotarget, 9 (2018) 5892-5905), the contents of which is herein incorporated by reference in its entirety. In this transduction model, in which oncogenes (AKT/YAP) are instilled directly into the biliary tree, tumors arise from the biliary tract in immunocompetent hosts with species-matched tumor microenvironment. Dosing is described in Table 9.

TABLE 9
Dosing

| | | | | | Route of | | Total |
|---|---|---|---|---|---|---|---|
| | | | Dose | Dose | Administration | | Number |
| Group | -n- | Test Agent | (μg/mouse) | Volume | (ROA) | Schedule | of Doses |
| 1 | 10 | Control Untreated | — | — | — | — | — |
| 2 | 10 | Control mIgG2 | 200 μg | 200 μl | IV | Q4Dx6 | 6 |
| 3 | 10 | Control mIgG2 | 400 μg | 200 μl | IV | Q4Dx6 | 6 |
| 4 | 10 | Control mIgG2 | 200 μg | 200 μl | IP | BIWx4 | 8 |
| 5 | 10 | Anti-Gal9 mIgG2 | 200 μg | 200 μl | IV | Q4Dx6 | 6 |
| | | (G9.2-17) | | | | | |
| 6 | 10 | Anti-Gal9 mIgG2 | 400 μg | 200 μl | IV | Q4Dx6 | 6 |
| | | (G9.2-17) | | | | | |
| 7 | 10 | Anti-Gal9 mIgG2 | 200 μg | 200 μl | IV | Q4Dx6 | 6 |
| | | (G9.1.8-m13) | | | | | |
| 8 | 10 | Anti-Gal9 mIgG2 | 400 μg | 200 μl | IV | Q4Dx6 | 6 |
| | | (G9.1.8-m13) | | | | | |

In brief, murine CCA cells (described in S. Rizvi, et al) are harvested and washed in DMEM. Male C57BL/6 mice from Jackson Labs are anesthetized using 1.5-3% isoflurane. Under deep anesthesia, the abdominal cavity is opened by a 1 cm incision below the xiphoid process. A sterile cotton tipped applicator is used to expose the superolateral aspect of the medial lobe of the liver. Using a 27-gauge needle, 40 μL of standard media containing 1×10\hat{ }6 cells is injected into the lateral aspect of the medial lobe. Cotton tipped applicator is held over the injection site to prevent cell leakage and blood loss. Subsequently, the abdominal wall and skin are closed in separate layers with absorbable chromic 3-0 gut suture material.

Two weeks post implantation, animals are matched by tumor volume into treatment or control groups to be used for dosing and dosing initiated on Day 0. Tumor volumes are measured and animals weighed three times weekly. A final tumor volume and weight is taken on the day the study reaches endpoint (4 weeks or when tumor burden of control becomes 1500 mm3). Blood is collected from all animals from each group.

### Example 11: In Vitro and In Vivo Characterization of Anti-Gal9 Antibody G9.2-17

In vivo and in vitro pharmacodynamics and pharmacology studies and safety pharmacology were conducted as disclosed below. In vivo studies were conducted with an IgG1 version of anti-galectin-9 mAb G9.2-17 for mouse studies based on the fact that this antibody was developed to have the exact same V_{H} and V_{L} chains and thus the exact same binding epitope as G9.2-17 and the same cross reactivity profile as well as binding affinities across species and same functional profile like G9.2-17.

### In Vitro Studies

G9.2-17 has multi-species cross-reactivity (human, mouse, rat, cynomolgus monkey), with equivalent <1 nmol binding affinities, as assessed in vitro. See, e.g., PCT/US2020/024767, the relevant disclosures of which are incorporated by reference for the subject matter and purpose as referenced herein. G9.2-17 does not cross react with the CRD1 domain of galectin-9 protein. It has excellent stability and purification characteristics, and no cross-reactivity to any of the other galectin proteins that exist in primates.

Table 10 below summarizes results from in vitro pharmacology studies.

TABLE 10
In Vitro Primary Pharmacodynamics

| Objectove | Assays | Key Results |
|---|---|---|
| Bead based | Binding of G9.2-17 to | Bead based measurements of G9.2-17 binding to the |
| binding - human | CRD1 and CRD2 | human galectin-9 CRD1 and CRD2 domains show |
| | domain of human | that G9.2-17 is specific to only the human CRD2 |
| | galectin-9 | domain of galectin-9. The mouse IgG1 version of |
| | | G9.2-17 show similar specificity to only the CRD2 |
| | | domain of galectin-9. KD Values (nM): G9.2-17 = |
| | | 0.15 ± 0.02, G9.2-17 mIgG1 = 0.18 ± 0.02. |
| Bead based | Binding of G9.2-17 to | Bead based measurements of G9.2-17 binding to the |
| binding - mouse | CRD2 domain of mouse | mouse galectin-9 CRD2 domain show that G9.2-17 |
| | galectin-9 | binds with <1 nMol to the mouse CRD2 domain. |
| | | The mouse IgG1 version of G9.2-17 show similar |
| | | affinity to the CRD2 domain of mouse galectin-9. |
| | | KD Values (nM): G9.2-17 = 0.30 ± 0.03; G9.2-17 |
| | | mIgG1 = 0.30 ± 0.1. |
| Bead based | Binding of G9.2-17 to | Bead based measurements of G9.2-17 binding to the |
| binding - rat | CRD2 domain of rat | rat galectin-9 CRD2 domain show that G9.2-17 binds |
| | galectin-9 | with <1 nMol to the rat CRD2 domain. The mouse |
| | | IgG1 version of G9.2-17 show similar affinity to the |
| | | CRD2 domain of rat galectin-9. KD Values (nM): |
| | | KD Values (nM): G9.2-17 = 0.31 ± 0.06; G9.2-17 |
| | | mIgG1 = 0.35 ± 0.06. |
| Bead based | Binding of G9.2-17 to | Bead based measurements of G9.2-17 binding to the |
| binding - | CRD2 domain of | cynomolgus galectin-9 CRD2 domain show that |
| monkey | cynomolgus monkey | cynomolgus G9.2-17 binds with <1 nMol to the |
| | galectin-9 | cynomolgus CRD2 domain. The mouse IgG1 version |
| | | of G9.2-17 show similar affinity to the CRD2 domain |
| | | of cynomolgus galectin-9. KD Values (nM): |
| | | G9.2-17 = 0.31 ± 0.03; G9.2-17 mIgG1 = 0.30 ± 0.10. |
| Binding - ELISA | ELISA based binding | G9.2-17 binding to human Galectin-9 CRD2 was |
| | assessment of G9.2-17 | assessed in ELISA format over a concentration |
| | to human CRD2 domain | range. G9.2-17 was titrated over immobilized |
| | of galectin-9 | Galectin-9 CRD2 and the resultant saturation curve |
| | | indicates that G9.2-17 has +211nMol to the CRD2 |
| | | domain of galectin-9. |
| | | The mouse IgG1 version of G9.2-17 show similar |
| | | affinity to the CRD2 domain of galectin-9 when |
| | | assayed in this format. |
| | | KD Values (nM): G9.2-17 = 0.42 ± 0.07; G9.2-17 |
| | | mIgG1 = 0.45 ± 0.04. |
| Binding - SPR | SPR based binding | SPR measurements using the One Step method on a |
| human | assessment of G9.2-17 | Pioneer SPR showed high binding of G9.2-17 to |
| | to human CRD2 domain | human galectin-9 CRD2. The resultant binding |
| | of galectin-9 | between the antibody and immobilized human |
| | | galectin-9 CRD2 had no measurable off rate even |
| | | after continued dissociation for over 30 minutes. |
| | | This suggests that G9.2-17 has a KD below the |
| | | measurable limit of assay. The mouse IgG1 version |
| | | of G9.2-17 showed similar behavior, with no |
| | | measurable off rate even over an extended |
| | | dissociation time. KD Values (nM): G9.2-17 = below |
| | | limit of detection; G9.2-17 mIgG1 = below limit of |
| | | detection. |
| Binding - SPR | SPR based binding | SPR measurements using the One Step method on a |
| mouse | assessment of G9.2-17 | Pioneer SPR showed high binding of G9.2-17 to |
| | to mouse CRD2 | domain mouse galectin-9 CRD2. Binding of G9.2-17 |
| | of galectin-9 | to mouse galectin-9 CRD2 had a KD value of 1.8 ± |
| | | 0.4 nM. The mouse IgG1 version of G9.2-17 showed |
| | | similar behavior, with a KD- value of 3.05 ± 0.03 nM. |
| | | KD Values (nM): G9.2-17 = 1.8 ± 0.4; G9.2-17 |
| | | mIgG1 = 3.05 ± 0.03. |
| Binding - Cell- | Assessment of cell | An assessment of G9.2-17 binding to galectin-9 on |
| based | surface based (CRL- | the cell surface was performed using the galectin-9 |
| | 2134 cell line) binding | positive CRL-2134 cell line. First, staining of |
| | of G9.2-17 | CRL2134 with G9.2-17 showed increased signal |
| | | compared to staining of the galectin-9 negative HEK- |
| | | 293 cell line. A saturation curve was then generated |
| | | by titrating G9.2-17 for surface staining of CRL- |
| | | 2134 cells. The curve was generated based on the |
| | | fraction of the cell population that were positive for |
| | | galectin-9 as compared unstained cells. Using the |
| | | generated saturation curve, a cell based KD of |
| | | 0.41 ± 0.07 nM was calculated. This assay was also |
| | | performed with the mouse IgG1 variant of G9.2-17 |
| | | with a resulting cell-based KD of 2.9 ± 0.7 nM. |
| Cell-based | G9.2-17 potency | MOLM-13 cells are sensitive to high concentrations |
| potency | assessment using | of human galectin-9. Incubation of MOLM-13 cells |
| T-cell apoptosis | MOLM-13 T cell-based | for 16 h in the presence of 200 nM galectin-9 results |
| | apoptosis assay | in significant cell death. The addition of G9.2-17 |
| | | protects MOLM-13 from galectin-9 mediated cell |
| | | death in a dose dependent manner, significantly |
| | | reducing the population of necrotic cells. This effect |
| | | is specific for G9.2-17 as well as the mouse IgG1 |
| | | variant of G9.2-17 while the matched human IgG4 |
| | | and mouse IgG1 isotypes show no protection against |
| | | galectin-9 mediated cell death. |
| Non-cell based | G9.2-17 potency | The receptor-ligand interaction between CD206 and |
| potency | assessment using non- | galectin-9 was assayed in ELISA format. Full length |
| T-cell apoptosis | cell based, competition | galectin-9 was immobilized and recombinant, His- |
| | ELISA CD206 | tagged CD206 was titrated to confirm CD206 does |
| | assay | bind to galectin-9. In order to determine whether or |
| | | not G9.2-17 blocked the binding between galectin-9 |
| | | binding and its native receptor CD206, a competitive |
| | | ELISA assay was utilized. Blockade of the galectin-9- |
| | | CD206 interaction resulted in reduced ELISA signal |
| | | compared to the unblocked condition in a dose |
| | | dependent manner. |
| Functional assay: | Bead based | G9.2-17 does not mediate ADCC or ADCP activity. |
| non-cell based | ADCC/ADCP assay | |
| ADCC/ADCP | | |
| assay | | |
| Protein array - | Protein Array - Cross | HuProt ™ array was used for the High-Spect |
| cross reactivity | reactivity | antibody cross-reactivity assay. Arrays contained |
| | | native and not denatured proteins. G9.2-17 |
| | | recognized galectin-9 (CDI clone or the positive |
| | | control antigen) as the top hit with high affinity. |
| Expression | Assessing cell surface | Dose dependent effect was observed in detection of |
| | and intra-cellular | cell surface galectin-9 on KPC cells, peaking at 20% |
| | galectin-9 levels by flow | using 60 nM G9.2-17 Fab. Intracellular galectin-9 |
| | cytometry on | expression was uniformly detected in 1000 of the |
| | permeabilized and non | cells at 15 nM, 30 nM and 60 nM of G9.2-17 Fab. |
| | permeabilized mouse | |
| | pancreatic cancer (KPC) | |
| | cells | |
| Expression | Assessing cell surface | 27.6% of B16F10 express galectin-9 on their surface |
| | and intra-cellular | and 98.8% intracellularly. 6.9% of MC38 express |
| | galectin-9 levels by flow | galectin-9 on their surface and 41.5% intracellularly. |
| | cytometry on | |
| | permeabilized and non | |
| | permeabilized mouse | |
| | melanoma (B16F10) and | |
| | colorectal cancer | |
| | (MC38) cells | |
| Mechanism of | Patient derived tumor | Activation of T cells measured through IFNg, TNFα |
| Action | cultures ex vivo | and CD44. n = 20 tumors processed. T cell |
| | (organoids) treated with | reactivation from baseline observed in n = 12 out of |
| | G9.2-17 | 20 (60%) of tumors processed. |
| Expression | Patient derived tumor | T cells galectin-9 expression (12.5-63.7% |
| | cultures ex vivo | CD3+CD45+ intra PTOD T cells). Myeloid cell |
| | (organoids) profiling for | galectin-9 expression (15-45.9% CD45+CD11b+ |
| | galectin-9 expression on | intra PDOT myelod cells). Tumor cell galectin-9 |
| | T cells, tumor cells and | expression (9.15-33.5% CD45-EpCAM+ intra PDOT |
| | macrophages | tumor cells) n = 6 PDOTs |
| Expression | Measuring galectin-9 | Sera from healthy controls (n = 16) and cancer |
| | levels in serum of | patients (n = 22; n = 10 primary and n = 12 |
| | healthy controls and | metastatic) with gastrointestinal malignancies. |
| | cancer patients | Galectin-9 serum levels are significantly increased in |
| | | cancer patients vs controls (p = 0.001) |
| Expression | Measuring galectin-9 | Sera and plasma from healthy controls (n = 10) and |
| | levels in serum and | cancer patients (n = 10) with metastatic tumors of |
| | plasma of healthy | diverse site of origin was tested for galectin-9 |
| | controls and cancer | expression. |
| | patients | |

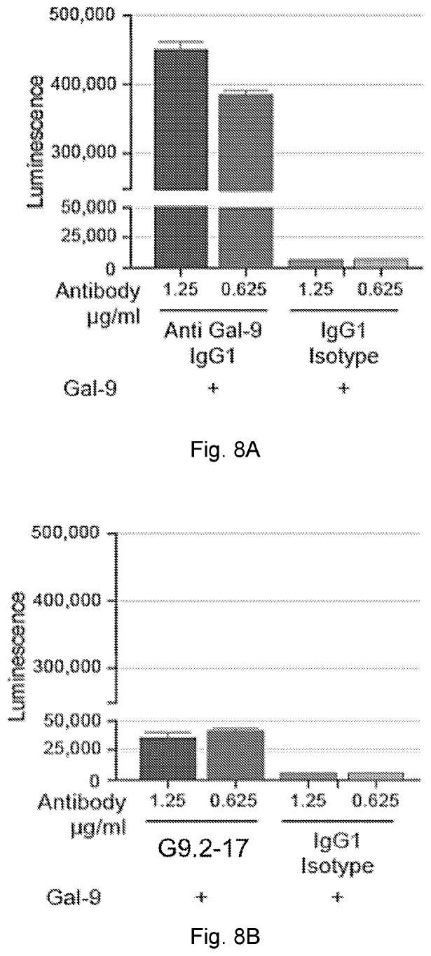

Studies to understand the mechanism of action included ADCC/ADCP (antibody dependent cell mediated cytotoxicity/antibody-dependent cellular phagocytosis) and blocking function assessment. As expected for a human IgG4 mAb, G9.2-17 does not mediate ADCC or ADCP (FIG. 8A). This was tested against the IgG1 human counterpart of G9.2-17 as a positive control, which mediates ADCC and ADCP, as expected (FIG. 8B).

Furthermore, blocking function of G9.2-17 was evaluated in a competition binding ELISA assay. G9.2-17 potently blocks binding of galectin-9 CRD2 domain to its binding partner CD206 human recombinant protein, confirming the intended mode of action for G9.2-17, which is to block galectin-9 activity. Moreover, we optimized a MOLM-13 T cell apoptosis assay where G9.2-17 proficiently rescues the cells from apoptosis caused by galectin-9 protein treatment (˜50% apoptosis with galectin-9 treatment and ˜10% apoptosis with galectin-9+G9.2-17 treatment).

Further extensive in vitro characterization has been done to compare binding and functional characteristics of G9.2-17 to the mouse IgG1 G9.2-17 mAb, which contains exactly the same CDR domains as G9.2-17, hence has the same binding epitope, i.e., CRD2 galectin-9 domain. mIgG1 G9.2-17 was developed for use in mouse syngeneic pharmacology efficacy studies, to avoid any potential development of immunogenicity with G9.2-17 itself. mIgG1 G9.2-17 has equivalent <1 nmol affinity across species, as well as the same cell based binding affinity to human cancer cell line, CRL-2134. mIgG1 G9.2-17 produces equivalent data in the MOLM-13 T cell apoptosis assay, as G9.2-17 itself.

### In Vivo Pharmacology

In vivo assays include syngeneic mouse models conducted using a mouse mAb —G9.2-17 binding epitope cloned into an IgG1 mouse backbone (G9.2-17 surrogate mAb for animal efficacy studies), which shares the cross reactivity and binding affinity characteristics of G9.2-17.

Syngeneic mouse models tested were:
Orthotopic pancreatic adenocarcinoma (KPC) mouse model (single agent and in combination with anti-PD-1): tumor volume assessment and flow cytometry;
Subcutaneous melanoma B16F10 model (single agent and in combination with anti-PD-1): tumor volume assessment and flow cytometry.
Subcutaneous MC38 model (single agent and in combination with anti-PD-1): tumor volume assessment

Further, patient-derived tumor cultures ex vivo (organoids) treated with G9.2-17 are to be used for exploring mechanism of action of G9.2-17.

Mechanistically, G9.2-17 was found to have blocking activity and not ADCC/ADCP activity. Blocking of galectin-9 interactions with its binding receptors, such as CD206 on immunosuppressive macrophages, is observed. Functionally, in vivo studies demonstrated reduction of tumor growth in multiple syngeneic models treated with G9.2-17 mIgG1 surrogate antibody (orthotopic pancreatic KPC tumor growth and s.c. melanoma B16F10 model). In mouse tumors treated with single agent anti-galectin-9 mAb and in combination with anti-PD-1, G9.2-17 reactivates effector T cells and reduces levels of immunosuppressive cytokines. Combination studies with an anti-PD-1 mAb suggest higher intra-tumoral presence of effector T cells, supporting clinical testing of the combinatorial approach. Importantly, mechanistic effects of G9.2-17 have been investigated and demonstrated in patient derived tumor cultures (Jenkins et al., 2018) (tumor excisions from primary and metastatic sites from PDAC, CRC, CCA, HCC), where G9.2-17 induces reproducible and robust T cell reactivation, indicating reversal of galectin-9 imposed intra-tumoral immunosuppression ex vivo.

In order to assess relevance of combining anti-PD-1 and anti-galectin-9 mAbs, s.c. melanoma B16 model was treated with single agent anti-PD-1 and anti-galectin-9 as well as the combination. Intra-tumoral presence effector T cells were enhanced in the combination arm.

Significant increases in the level of cytotoxic T cells (CD8) are observed in treatments with anti-galectin-9 mIgG1 200 μg+anti-PD-1 (p<0.001) compared to that of anti-galectin-9 mIgG1 200 μg, and between anti-galectin-9 IgG1 200 μg+anti-PD-1 compared to anti-PD-1 alone (p<0.01). Such results suggest that anti-Gal9 antibody and anti-PD-1 antibody in combination would be expected to achieve superior therapeutic effects.

Table 11 below summarizes results from in vivo pharmacology studies.

TABLE 11
In Vivo Primary Pharmacodynamics

| Study Title | Test System | Key Results |
|---|---|---|
| Efficacy study assessing tumor | Orthotopic | Efficacy observed with single agent IgG1 |
| volume and flow cytometry of intra- | KPC model | mouse galectin-9 mAb, p = 0.05. Flow |
| tumoral immune cells in mice | | cytometry: CD8 T cells: Increase in CD8+ |
| treated with IgG1 mouse anti- | | T cell TNF alpha (p = 0.027), increase in |
| galectin-9 mAb at 150 μg/dose i.p. | | CD8+ T cell CD44 (p = 0.0008) and |
| | | reduction in CD8+ T cell IL10 (p = 0.0026). |
| | | Increase in CD4+ T Cell TNF alpha (p = |
| | | 0.0007). |
| Efficacy study assessing tumor | Orthotopic | Efficacy observed at 200 i.ig (p = 0.0005) |
| volume and flow cytometry of intra- | KPC model | and 400 μg (p = 0.01) dose levels of single |
| tumoral immune cells in mice | | agent anti-galectin-9 mIgG1 mAb. Flow |
| treated with IgG1 mouse anti- | | cytometry: CD8+ T cells: increase of CD44 |
| galectin-9 mAb at 200 and 400 | | (for dose levels 200 μg and 400 μg p = |
| μg/dose i.p. | | 0.002). CD4+ T cells: Increase in CD44 (for |
| | | dose level 200 μg, p = 0.015 and for dose |
| | | level 400 μg p = 0.0003). |
| Efficacy study assessing tumor | orthotopic | Efficacy observed at both dose levels (p < |
| volume and flow cytometry of intra- | KPC model | 0.01). Flow cytometry: CD4+ T cells: |
| tumoral immune cells in mice | | increase in CD44 (p < 0.0001), PD-1 (for |
| treated with IgG1 mouse anti- | | dose level 100 μg p = 0.005 and for dose |
| galectin-9 mAb at 100 and 200 | | level 200 μg p = 0.001); CD8+ T cells: |
| μg/dose i.p. | | increase in CD44 (p < 0.0001). |
| Efficacy study assessing tumor | Orthotopic | Efficacy observed at 50 μg (p < 0.05) and |
| volume in mice treated with IgG1 | KPC model | 100 μg (p < 0.0001) dose levels and no |
| mouse anti-galectin-9 mAb at 20, | | significant efficacy at 20 μg/dose. No |
| 50 and 100 μg/dose i.p. + 100 | | significant TV synergy effect with |
| μg/dose IgG1 mouse anti-galectin-9 | | combination of 100 μg anti-galectin-9 mAb |
| mAb with anti-PD-1 | | and anti-PD-1 |
| Efficacy study assessing tumor | Sub cutaneous | Highest efficacy observed at 200 i.is (p < |
| volume and flow cytometry in mice | B16F10 | 0.005) single agent mouse anti-galectin-9 |
| treated with IgG1 mouse anti- | model | mAb superior to anti-PD-1 mAb. No |
| galectin-9 mAb at 200 and 400 | | significant TV synergy effect with |
| μg/dose i.v. + anti-PD-1 mAb | | combination of 200 μg anti-galectin-9 mAb |
| | | and anti-PD-1 on tumor growth. However, |
| | | significant increase in cytotoxic CD8 T cell |
| | | levels were observed in mouse anti-galectin- |
| | | 9 mAb + anti-PD-1 mAb (p < 0.01). |
| Efficacy study assessing tumor | Sub cutaneous | Efficacy not superior to anti-PD-1 mAb in |
| volume in mice treated with IgG1 | MC38 model | this model. Combination with anti-PD-1 is |
| mouse anti-galectin-9 mAb at 200 | | equivalent to anti-PD-1 alone. Please refer to |
| and 400 μg/dose i.v. + anti-PD-1 | | CFCH001 for flow cytometry data |
| mAb | | explaining low expression of galectin-9 on |
| | | MC38 cells. |

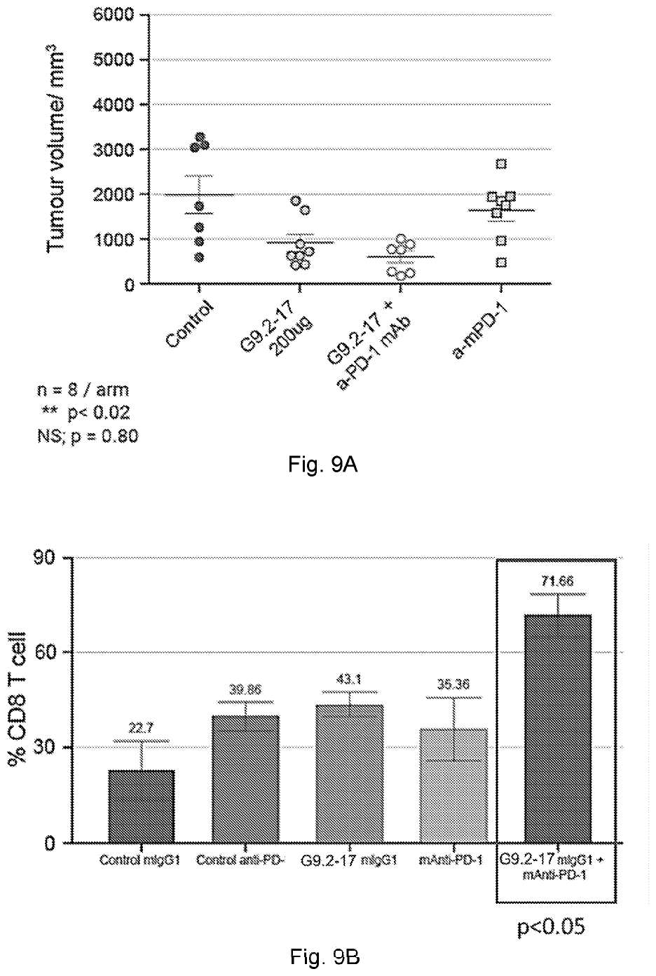

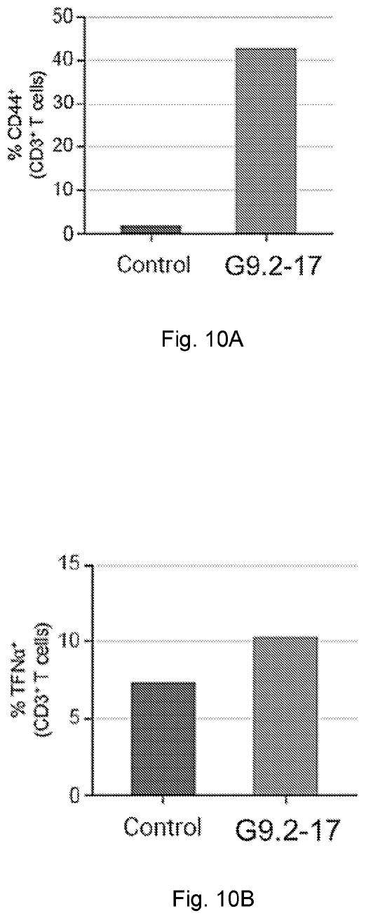

Further, tumor immune responses to treatment with G9.2-17 IgG1 mouse mAb (aka G9.2-17 mIgG), anti-PD1 antibody, or a combination of the G9.2-17 IgG1 mouse mAb and anti-PD1 antibody were investigated in the B16F10 subcutaneous syngeneic model described herein. As shown in FIG. 9A and FIG. 9B, the G9.2-17 and anti-PD1 combination showed synergistic effects in reducing tumor volume and in increasing CD8+ cells in the mouse model. FIGS. 10A and 10B show that the G9.2-17 antibody increased CD44 and TNFα expression in intratumoral T cells.

### Example 12. A Non-GLP Single-Dose, Range-Finding Intravenous Toxicity Study in Male Sprague Dawley Rats with 1- and 3-Week Postdose Observation Periods

This study evaluated the anatomical endpoints of G9.2-17 IgG4 following a single intravenous bolus administration to Sprague Dawley rats followed by 1-week (terminal) and 3-week (recovery) necropsies on Days 8 and 22. All animals survived to the scheduled necropsies. There were no test article-related macroscopic findings, organ weight changes, or microscopic findings in either the terminal or recovery necropsy animals on this study.

The objective of this non-GLP exploratory, single-dose, range finding, intravenous toxicity study was to identify and characterize the acute toxicities of G9.2-17 IgG4 following intravenous bolus administration over 2 minutes to Sprague Dawley rats followed by 1-week (terminal) and 3-week (recovery) postdose observation periods.

This non-GLP single dose toxicity study was conducted in 24 Sprague Dawley male rats to determine the toxicokinetics and potential toxicity of G9.2-17 IgG4. Animals were administered either vehicle or 10 mg/kg, 30 mg/kg or 70 mg/kg G9.2-17 IgG4 by slow bolus intravenous injection for at least 2 minutes on Day 1 followed by either a 1-week (terminal, Day 8) or 3-week (recovery, Day 22) period after the dose. Study endpoints included mortality, clinical observations, body weights, and food consumption, clinical pathology (hematology, coagulation, clinical chemistry and urinalysis), toxicokinetic parameters, ADA evaluation and anatomic pathology (gross necropsy, organ weights, and histopathology). Summaries of the experimental design is provided in Table 13 below.

TABLE 13
Experimental Design

| Group | | Dosage Level | |
|---|---|---|---|
| Number | Treatment | (mg/kg) | Number of Males ^{a} |
| 1 | Vehicle ^{b} | 0 | 6 |
| 2 | G9.2-17 IgG4 | 10 | 6 |
| 3 | G9.2-17 IgG4 | 30 | 6 |
| 4 | G9.2-17 IgG4 | 70 | 6 |

^{a} 3 animals/sex/group were euthanized at the Day 8 terminal necropsy; the remaining 3 animals/sex/group were euthanized at the Day 22 recovery necropsy.
^{b} The vehicle was Formulation Buffer (20mM Tris, 150 mM NaCl, pH 8.0 ± 0.05).

All surviving animals were submitted for necropsy on Day 8 or Day 22. Complete postmortem examinations were performed and organ weights were collected. The organs were weighed from all animals at the terminal and recovery. Tissues required for microscopic evaluation were trimmed, processed routinely, embedded in paraffin, and stained with hematoxylin and eosin.

There were no unscheduled deaths during the course of this study. All animals survived to the terminal or recovery necropsies. Histological changes noted were considered to be incidental findings or related to some aspect of experimental manipulation other than administration of the test article. There was no test article related alteration in the prevalence, severity, or histologic character of those incidental tissue alterations. No G9.2-17 IgG4-related findings were noted in clinical observations, body weights, food consumption, clinical pathology or anatomic pathology. In conclusion, the single intravenous administration of 10, 30, and 70 mg/kg G9.2-17 IgG4 to Sprague Dawley rats was tolerated with no adverse findings. Therefore, under the conditions of this study the NOEL was 70 mg/kg.

### Example 13. A Non-GLP Single-Dose, Range-Finding Intravenous Infusion Toxicity Study of G9.2-17 IgG4 in Cynomolgus Monkeys with a 3-Week Post-Dose Observation Period

This non-GLP single-dose toxicity study was conducted in 8 cynomolgus monkeys to identify and characterize the acute toxicities of G9.2-17 IgG4. Animals (1 male [M]/1 female [F]/group) were administered either vehicle or 30 mg/kg, 100 mg/kg, or 200 mg/kg G9.2-17 IgG4 by 30-minute intravenous (IV) infusion followed by a 3 week post-dose observation period. Study endpoints included: mortality, clinical observations, body weights, and qualitative food consumption; clinical pathology (hematology, coagulation, clinical chemistry, immunophenotyping and galectin 9 expression on leukocyte subsets, and cytokine analysis); toxicokinetic parameters; serum collection for possible anti-drug antibody evaluation (ADA); and soluble galectin-9 analyses; and anatomic pathology (gross necropsy, organ weights, and histopathology).

No G9.2-17 IgG4-related findings were noted in clinical observations, body weights, food consumption, clinical pathology (hematology, clinical chemistry, coagulation, or cytokine analysis), immunophenotyping, galectin-9 expression on leukocyte subsets, soluble galectin-9 or anatomic pathology.

In conclusion, the single intravenous infusion administration of 30, 100, and 200 mg/kg G9.2-17 IgG4 to cynomolgus monkeys was tolerated with no adverse findings. Therefore, under the conditions of this study the No-observed-Adverse-Effect-Level (NOAEL) was 200 mg/kg, the highest dose level evaluated. The study design is shown in Table 14.

TABLE 14
Experimental Design

| | | | | | | Dose Volume | | Animal |
|---|---|---|---|---|---|---|---|---|
| | | | | Adjusted Dose | | (mL/kg) | | No. |
| Group | | Dose Level | | Concentration (mg/mL) | | Necropsy | | Necropsy |
| No. | Treatment | (mg/kg) | | | Males | Day | Females | Day |
| 1 | Vehicle | 0 | 0 | 20 | 1001 | 22 | 1501 | 22 |
| 2 | G9.2-17 | 30 | 1.5 | 20 | 2001 | 22 | 2501 | 22 |
| | IgG4 | | | | | | | |
| 3 | G9.2-17 | 100 | 5 | 20 | 3001 | 22 | 3501 | 22 |
| | IgG4 | | | | | | | |
| 4^{a} | G9.2-17 | 200 | 10 | 20 | 4001 | 22 | 4501 | 22 |
| | IgG4 | | | | | | | |
| | | Dose | Adjusted Dose | Dose | Animal No. | | | |
| Group | | Level | Concentration | Volume | | Necropsy | | Necropsy |
| No. | Treatment | (mg/kg) | (mg/mL) | (mL/kg) | Males | Day | Females | Day |
| 1 | Vehicle | 0 | 0 | 20 | 1001 | 22 | 1501 | 22 |
| 2 | G9.2-17 | 30 | 1.5 | 20 | 2001 | 22 | 2501 | 22 |
| | IgG4 | | | | | | | |
| 3 | G9.2-17 | 100 | 5 | 20 | 3001 | 22 | 3501 | 22 |
| | IgG4 | | | | | | | |
| 4^{a} | G9.2-17 | 200 | 10 | 20 | 4001 | 22 | 4501 | 22 |
| | IgG4 | | | | | | | |

^{a}Group 4 was administered 1 week after administration of Groups 1 through 3.

The vehicle and test article were administered once via IV infusion for 30 minutes during the study via a catheter percutaneously placed in the saphenous vein. The dose levels were 30, 100, and 200 mg/kg and administered at a dose volume of 20 mL/kg. The control group received the vehicle in the same manner as the treated groups.

The animals were placed in sling restraints during dosing. The vehicle or test article were based on the most recent body weights and administered using an infusion pump and sterile disposable syringes. The dosing syringes were filled with the appropriate volume of vehicle or test article (20 mL/kg with 2 mL extra). At the completion of dosing, the animals were removed from the infusion system. The weight of each dosing syringe was recorded prior to the start and end of each infusion to determine dose accountability.

Detailed Clinical Observations

The animals were removed from the cage, and a detailed clinical examination of each animal was performed at 1 and 4.5 hours post-start of infusion (SOI) on Day 1 and once daily thereafter during the study. The animals were removed from the cage, and a detailed clinical examination of each animal was performed at 1 and 4.5 hours post-start of infusion (SOI) on Day 1 and once daily thereafter during the study. Body weights for all animals were measured and recorded at transfer, prior to randomization, on Day −1, and weekly during the study.

Clinical pathology evaluations (hematology, coagulation, and clinical chemistry) were conducted on all animals pretest and on Days 1 (prior to dosing), 3, 8, and 21. Additional samples for the determination of hematology parameters and peripheral blood lymphocyte and cytokine analysis samples were collected at 30 minutes (immediately after the end of infusion) and 4.5, 8.5, 24.5, and 72.5 hours post-SOI (relative to Day 1). Bone marrow smears were collected and preserved.

Blood samples (approximately 0.5 mL) were collected from all animals via the femoral vein for determination of the serum concentrations of the test article (see Table 15) (for a deviation, see Appendix 1). The animals were not fasted prior to blood collection, with the exception of the intervals that coincided with fasting for clinical pathology collections.

TABLE 15
Bioanalysis Sample Collection Schedule

| | Sample Collection Time Points (Time Post-SOI) relative to Day 1 | | | | | | | | | | | | |
|---|---|---|---|---|---|---|---|---|---|---|---|---|---|
| Group | | 0.583 | 1 | 2.5 | 4.5 | 8.5 | 4.5 hr | 48.5 hr | 72.5 hr | 120.5 hr | 168.5 hr | 360.5 hr | 504.5 hr |
| No. | Predose | hr^{a} | hr | hr | hr | hr | (Day 2) | (Day 3) | (Day 4) | (Day 6) | (Day 8) | (Day 16) | (Day 22) |
| 1-4 | X | X | X | X | X | X | X | X | X | X | X | X | X |

X = Sample was collected.
^{a}Only the 0.583 hr post-SOI timepoint from Group 1 animals was analyzed for test article content. Additional timepoints may be analyzed at the discretion of the Study Director.

For processing, blood samples were collected in non-additive barrier free microtubes and centrifuged at controlled room temperature within 1 hour of collection. The resulting serum was divided into 2 approximately equal aliquots in pre labeled cryovials. All aliquots were stored frozen at −60° C. to −90° C. within 2 hours of collection.

Postmortem study evaluations were performed on all animals euthanized at the scheduled necropsy.

Necropsy examinations were performed under procedures approved by a veterinary pathologist. The animals were examined carefully for external abnormalities including palpable masses. The skin was reflected from a ventral midline incision and any subcutaneous masses were identified and correlated with antemortem findings. The abdominal, thoracic, and cranial cavities were examined for abnormalities. The organs were removed, examined, and, where required, placed in fixative. All designated tissues were fixed in neutral buffered formalin (NBF), except for the eyes (including the optic nerve) and testes. The eyes (including the optic nerve) and testes were placed in a modified Davidson's fixative, and then transferred to 70% ethanol for up to three days prior to final placement in NBF. Formalin was infused into the lung via the trachea. A full complement of tissues and organs was collected from all animals.

Body weights and protocol-designated organ weights were recorded for all animals at the scheduled necropsy and appropriate organ weight ratios were calculated (relative to body and brain weights). Paired organs were weighed together. A combined weight for the thyroid and parathyroid glands was collected.

Results

All animals survived to the scheduled necropsy on Day 22. No test article-related clinical or veterinary observations were noted in treated animals. No test article-related effects on body weight were observed in treated animals during the treatment or recovery period. There were no G9.2-17 IgG4-related effects on hematology endpoints in either sex at any dose level at any interval.

There were no G9.2-17 IgG4-related effects on coagulation times (i.e., activated partial thromboplastin times [APTT] and prothrombin times) or fibrinogen concentrations in either sex at any dose level at any interval. All fluctuations among individual coagulation values were considered sporadic, consistent with biologic and procedure-related variation, and/or negligible in magnitude, and not related to G9.2-17 IgG4 administration.

There were no G9.2-17 IgG4-related effects on clinical chemistry endpoints in either sex at any dose level at any interval. All fluctuations among individual clinical chemistry values were considered sporadic, consistent with biologic and procedure-related variation, and/or negligible in magnitude, and not related to G9.2-17 IgG4 administration.

There were no G9.2-17 IgG4-related effects on cytokine endpoints in either sex at any dose level at any interval. All fluctuations among individual cytokine values were considered sporadic, consistent with biologic and procedure-related variation, and/or negligible in magnitude, and not related to G9.2-17 IgG4 administration.

Review of the gross necropsy observations revealed no findings that were considered to be test article related. There were no organ weight alterations that were considered to be test article-related. There were no test article-related changes.

In conclusion, the single intravenous infusion administration of 30, 100, and 200 mg/kg G9.2-17 IgG4 to cynomolgus monkeys was tolerated with no adverse findings.

Therefore, under the conditions of this study the No-observed-Adverse-Effect-Level (NOAEL) was 200 mg/kg, the highest dose level evaluated.

The animals were removed from the cage, and a detailed clinical examination of each animal was performed at 1 and 4.5 hours post-start of infusion (SOI) on Day 1 and once daily thereafter during the study.

### Example 12: Intravenous Infusion Study of G9.2-17 in Cynomolgus Monkeys

The objective of this study was to further characterize the toxicity and toxicokinetics of the test article, G9.2-17 (a hIgG4 Monoclonal Antibody which binds to Galectin-9), following once weekly 30-minute intravenous (IV) infusion for 5 weeks in cynomolgus monkeys, and to evaluate the reversibility, progression, or delayed appearance of any observed changes following a 3-week recovery period.

### Experimental Design

Table 12 summarizes the study design.

TABLE 12
Experimental Design

| | | | Dose | Dose | Main Study | | Recovery Study | |
|---|---|---|---|---|---|---|---|---|
| Group | Test | Dose Level | Volume^{a} | Concentration | No. of | No. of | No. of | No. of |
| No. | Material | (mg/kg/dose) | (mL/kg) | (mg/mL) | Males | Females | Males | Females |
| 1 | Vehicle | 0 | 10 | 0 | 3 | 3 | 2 | 2 |
| 2 | G9.2-17 | 100 | 10 | 10 | 3 | 3 | 2 | 2 |
| 3 | G9.2-17 | 300 | 10 | 30 | 3 | 3 | 2 | 2 |

^{a}Based on the most recent practical body weight measurement.

Animals (cynomolgus monkeys) used in the study were assigned to study groups by a standard, by weight, randomization procedure designed to achieve similar group mean body weights. Males and females were randomized separately. Animals assigned to study had body weights within ±20% of the mean body weight for each sex.

The formulations lacking G9.2-17 (“vehicle”) or encompassing G9.2-17 (“test article”) were administered to the animals once weekly for 5 weeks (Days 1, 8, 15, 22, and 29) during the study via 30-minute IV infusion. The dose levels were 0, 100 and 300 mg/kg/dose and administered at a dose volume of 10 mL/kg. The control animals group received the vehicle in the same manner as the treated groups. Doses were administered via the saphenous vein via a percutaneously placed catheter and a new sterile disposable syringe was used for each dose. Dose accountability was measured and recorded prior to dosing and at the end of dosing on toxicokinetic sample collection days (Days 1, 15, and 29) to ensure a ±10% target dose was administered. Individual doses were based on the most recent body weights. The last dose site was marked for collection at the terminal and recovery necropsies. All doses were administered within 8 hours of test article preparation.

In-life procedures, observations, and measurements were performed on the animals as exemplified below.

Electrocardiographic examinations were performed on all animals. Insofar as possible, care was taken to avoid causing undue excitement of the animals before the recording of electrocardiograms (ECGs) in order to minimize extreme fluctuations or artifacts in these measurements. Standard ECGs (10 Lead) were recorded at 50 mm/sec. Using an appropriate lead, the RR, PR, and QT intervals, and QRS duration were measured and heart rate was determined. Corrected QT (QTc) interval was calculated using a procedure based on the method described by Bazett (1920). All tracings were evaluated and reported by a consulting veterinary cardiologist.

To aid in continuity and reliability, functional observational battery (FOB) evaluations were conducted by two independent raters for all occasions and consisted of a detailed home cage and open area neurobehavioral evaluation (Gauvin and Baird, 2008). Each technician scored the monkey independently (without sharing the results with each other) for each home cage and out of cage observational score, and then the individual scores were assessed for agreement with their partner's score after the completion of the testing. FOB evaluations were conducted on each animal predose (on Day −9 or Day 8) to establish baseline differences and at 2 to 4 hours from the start of infusion on Days 1 and 15, and prior to the terminal and recovery necropsies. The observations included, but were not limited to, evaluation of activity level, posture, lacrimation, salivation, tremors, convulsions, fasciculations, stereotypic behavior, facial muscle movement, palpebral closure, pupil response, response to stimuli (visual, auditory, and food), body temperature, Chaddock and Babinski reflexes, proprioception, paresis, ataxia, dysmetria, and slope assessment, movement, and gait.

Blood pressure of each animal was measured and recorded and consisted of systolic, diastolic, and mean arterial pressure. Blood pressure measurements are reported using three readings that have the Mean Arterial Pressure (MAP) within 20 mmHg.

Respiratory rates of each animal were measured and recorded 3 times per animal/collection interval by visual assessment per Testing Facility SOP. The average of the 3 collections is the reported value.

Clinical pathology evaluations (e.g., immunophenotyping and cytokine evaluations) were conducted on all animals at predetermined intervals. Bone marrow smears were collected and preserved. Blood samples (approximately 0.5 mL) were collected from all animals via the femoral vein for determination of the serum concentrations of the test article. The animals were not fasted prior to blood collection, with the exception of the intervals that coincided with fasting for clinical pathology collections. At the conclusion of the study (day 36 or day 50), animals were euthanatized and tissues for histology processing and microscopic evaluation were collected.

Soluble galectin-9 was evaluated as follows. Blood samples (approximately 1 mL) were collected from all animals via the femoral vein for determination of the serum for soluble galectin 9 predose and 24 hours from the start of infusion on Days 1, 8, 15, and 29, and prior to the terminal and/or recovery necropsies. The animals were not fasted prior to blood collection, with the exception of the intervals that coincided with fasting for clinical pathology collections.

Soluble galectin-9 samples were processed as follows. Blood samples were collected in non-additive, barrier free tubes, allowed to clot at ambient temperature, and centrifuged at ambient temperature. The resulting serum was divided into 2 aliquots (100 μL in Aliquot 1 and remaining in Aliquot 2) in pre labeled cryovials. All aliquots were flash frozen on dry ice within 2 hours of collection and stored frozen at −60° C. to 90° C.

All results presented in the tables of the report were calculated using non-rounded values as per the raw data rounding procedure and may not be exactly reproduced from the individual data presented.

Results

Mortality

All animals survived to the scheduled terminal necropsy on Day 36 and recovery necropsy on Day 50.

Detailed Clinical and Veterinary Observations

No test article-related clinical or veterinary observations were noted in treated animals during the treatment or recovery periods.

Functional Observational Battery

No test article-related FOB observations were noted in treated animals during the treatment or recovery periods.

Body Weight and Body Weight Gains

No test article-related effects in body weight and body weight gain were noted in treated animals during the treatment or recovery periods.

Ophthalmology Examinations

No test article-related effects in ophthalmology examinations were noted in treated animals during the treatment or recovery periods.

Blood Pressure Values

No test article-related effects in blood pressure values were noted in treated animals during the treatment or recovery periods.

Respiratory Rate Values

No test article-related effects in respiratory rate values were noted in treated animals during the treatment or recovery periods.

Electrocardiology

No test article-related effects in electrocardiographic evaluations were noted in treated animals during the treatment or recovery periods.

Hematology

There were no G9.2-17-related effects among hematology parameters in either sex at any dose level at any timepoint.

Coagulation

There were no G9.2-17-related effects among coagulation parameters in either sex at any dose level at any timepoint.

Clinical Chemistry

There were no G9.2-17-related effects among clinical chemistry parameters in either sex at any dose level at any timepoint.

Urinalysis

No G9.2-17-related alterations were observed among urinalysis parameters in either sex at any dose level at the 13-week interim.

Cytokine

No definitive G9.2-17-related effects on cytokines were seen at any dose level or timepoint.

Peripheral Blood Leukocyte Analysis (PBLA)

There were no G9.2-17-related effects on PBLA endpoints in either sex at any dose level at any timepoint.

Bioanalysis, Galectin-9, and Toxicokinetic Evaluation

G9.2-17 was quantifiable in all cynomolgus monkey samples from all G9.2-17-dosed animals after dose administration. No measurable amount of G9.2-17 was detected in control cynomolgus monkey samples. Soluble galectin-9 was quantifiable in all cynomolgus monkey samples from all animals. G9.2-17 serum concentrations were below the bioanalytical limit of quantitation (LLOQ<0.04 ug/mL) in all serum samples obtained predose from most G9.2-17 treated animals on Day 1 and from control animals on Days 1 and 29.

Gross Pathology and Organ Weight

There were no definitive test article-related macroscopic observations in main study or recovery animals. There were also no test article-related organ weight changes for main study or recovery animals.

Histopathology

There were no definitive test article-related microscopic observations.

In conclusion, once weekly intravenous infusion administration of 100 and 300 mg/kg of G9.2-17 for 5-weeks to cynomolgus monkeys was tolerated with no adverse findings.

### Example 13: Intravenous Infusion Study of G9.2-17 in Sprague Dawley Rats

The objective of this study was to evaluate potential toxicity of G9.2-17, an IgG4 human monoclonal antibody directed against galectin-9, when administered by intravenous injection to Sprague Dawley Rats once weekly for 4 consecutive weeks followed by a 3-week post dose recovery period. In addition, the toxicokinetic characteristics of G9.2-17 were determined.

### Experimental Design

Table 13 summarizes the study design.

TABLE 13
Study Design

| | | Dose | Dose | Dose | | | | | | |
|---|---|---|---|---|---|---|---|---|---|---|
| | Test | Level | Concentration | Volume^{a} | Terminal | | Recovery | | TK/Gal-9/Cyto | |
| Group | Material | (mg/kg) | (mg/mL) | (mL/kg) | M | F | M | F | M | F |
| 1 | Control | 0 | 0 | 10 | 10 | 10 | 5 | 5 | 12 | 12 |
| 2 | G9.2-17 | 100 | 10 | 10 | 10 | 10 | 5 | 5 | 12 + 6^{b} | 12 + 6^{b} |
| 3 | G9.2-17 | 300 | 30 | 10 | 10 | 10 | 5 | 5 | 12 + 6^{b} | 12 + 6^{b} |

^{a}Individual dose volumes were calculated based on the most recent body weight.
^{b}SSD animals: 3 animals/sex/group for TK collections only following a single dose administration on Day 1.

One hundred eighty-six animals (Sprague Dawley rats) were assigned to treatment groups randomly by body weight. Control Article/Vehicle, Formulation Buffer for Test Article, and test article, G9.2-17, were administered via a single IV injection in a tail vein at dose levels of 0, 100, and 300 mg/kg once on Days 1, 8, 15, 22, and 29. Test article was administered at dose levels of 100 and 300 mg/kg once on Day 1 to animals assigned to the SSD subgroup.

Clinical observations were performed once daily prior to room cleaning in the morning, beginning on the second day of acclimation. A mortality check was conducted twice daily to assess general animal health and wellness. Food consumption was estimated by weighing the supplied and remaining amount of food in containers once weekly. The average gram (g)/animal/day was calculated from the weekly food consumption. Body weights were taken prior to randomization, on Day −1, then once weekly throughout the study, and on the day of each necropsy. Functional Observation Battery (FOB) observations were recorded for SSB animals approximately 24 hours post dose administrations on Days 1, 35 and 49. Urine was collected overnight using metabolic cages. Samples were obtained on Days 36 and 50.

Animals were fasted overnight prior to each series of collections that included specimens for serum chemistry. In these instances associated clinical pathology evaluations were from fasted animals. Blood was collected from a jugular vein of restrained, conscious animals or from the vena cava of anesthetized animals at termination.

Parameters assessed during the In-life examinations of the study included clinical observations, food consumption, body weights, functional observational battery. Blood samples were collected at selected time points for clinical pathology (hematology, coagulation, and serum chemistry) analyses. Urine samples were collected for urinalysis. Blood samples were also collected at selected time points for toxicokinetic (TK), immunogenicity (e.g., anti-drug antibody or ADA), and cytokine analyses. Animals were necropsied on Days 36 and 50. At each necropsy, gross observations and organ weights were recorded, and tissues were collected for microscopic examination.

### Results

### In-Life Examinations

Mortality: There were no abnormal clinical observations or body weight changes noted for this animal during the study.

Clinical Observations: There were no G9.2-17-related clinical observations noted during the study.

Food Consumption/Body Weights: There were no G9.2-17-related changes in food consumption, body weights or body weight gain noted during the study.

Clinical Pathology: There were no G9.2-17-related changes noted in clinical pathology parameters.

Cytokine Analysis: There were no G9.2-17-related changed in serum concentrations of IL-2, IL-4, IFN-γ, IL-5, IL-6, IL-10, and/or TNF-α, MCP-1 and MIP-1b.

Gross Pathology: There were no G9.2-17-related gross observations. Further, were no G9.2-17-related changes in absolute or relative organ weights.

Histopathology: There were no G9.2-17-related histologic findings.

In conclusion, intravenous G9.2-17 administration to Sprague Dawley rats once weekly for a total of 5 doses was generally well tolerated. There were no G9.2-17-related changes in clinical observations, food consumption, body weights, FOB parameters, clinical pathology, cytokine, gross observations, or organ weights.

### EQUIVALENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations are depend upon the specific application or applications for which the inventive teachings is/are used. Those skilled in the art are recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1.-44. (canceled)

45. A method for treating cancer, the method comprising administering to a subject in need thereof an effective amount of an antibody that binds human Galectin-9 (anti-Galectin-9 antibody), wherein the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6 and wherein the anti-Galectin-9 antibody is administered to the subject at a dose of about 1 mg/kg to about 32 mg/kg.

46. The method of claim 45, wherein the cancer is a solid tumor.

47. The method of claim 46, wherein the solid tumor is pancreatic cancer, colorectal cancer (CRC), hepatocellular carcinoma (HCC), or cholangiocarcinoma (CCA).

48. The method of claim 46, wherein the solid tumor is a metastatic tumor.

49. The method of claim 45, wherein the anti-Galectin-9 antibody is administered to the subject once every two weeks.

50. The method of claim 45, wherein the anti-Galectin-9 antibody is administered to the subject at a dose of about 1 mg/kg to about 16 mg/kg.

51. The method of claim 45, wherein the anti-Galectin-9 antibody is administered to the subject at a dose of about 1 mg/kg, about 2 mg/kg, about 4 mg/kg, about 6 mg/kg, about 8 mg/kg, about 10 mg/kg, about 12 mg/kg, or about 16 mg/kg.

52. The method of claim 51, wherein the anti-Galectin-9 antibody is administered to the subject by intravenous infusion.

53. The method of claim 52, wherein the subject is free of other anti-cancer therapy concurrently with the treatment involving the anti-Galectin-9 antibody.

54. The method of claim 53, wherein the method further comprises administering to the subject an immune checkpoint inhibitor.

55. The method of claim 54, wherein the immune checkpoint inhibitor is an antibody that binds PD-1.

56. The method of claim 55, wherein the antibody that binds PD-1 is selected from the group consisting of pembrolizumab, nivolumab, tislelizumab or cemiplimab.

57. The method of claim 56, wherein the antibody that binds PD-1 is nivolumab, which is administered to the subject at a dose of 240 mg once every two weeks.

58. The method of claim 56, wherein the antibody that binds PD-1 is tislelizumab, which is administered at a dose of 200 mg once every 3 weeks.

59. The method of claim 56, wherein the immune checkpoint inhibitor is administered by intravenous infusion.

60. The method of claim 45, wherein the anti-Galectin-9 antibody comprises a light chain variable domain of SEQ ID NO: 8, and a heavy chain variable domain of SEQ ID NO: 7.

61. The method of claim 60, wherein the anti-Galectin-9 antibody is a full-length antibody.

62. The method of claim 61, wherein the anti-Galectin-9 antibody is an IgG1 or IgG4 molecule.

63. The method of claim 62, wherein the anti-Galectin-9 antibody is an IgG4 molecule having a modified Fc region of human IgG4.

64. The method of claim 63, wherein the modified Fc region of human IgG4 comprises the amino acid sequence of SEQ ID NO: 14.

65. The method of claim 60, wherein the anti-Galectin-9 antibody comprises a heavy chain variable region comprising the amino acid sequence of SEQ ID NO: 7 and a light chain comprising the amino acid sequence of SEQ ID NO: 8.

66. The method of claim 45, wherein the anti-Galectin-9 antibody comprises a heavy chain comprising the amino acid sequence of SEQ ID NO: 19 and a light chain comprising the amino acid sequence of SEQ ID NO: 15.

67. The method of claim 66, wherein the cancer is a solid tumor.

68. The method of claim 67, wherein the solid tumor is pancreatic cancer, colorectal cancer (CRC), hepatocellular carcinoma (HCC), or cholangiocarcinoma (CCA).

69. The method of claim 67, wherein the solid tumor is a metastatic tumor.

70. The method of claim 67, wherein the anti-Galectin-9 antibody is administered to the subject once every two weeks.

71. The method of claim 70, wherein the anti-Galectin-9 antibody is administered to the subject at a dose of about 1 mg/kg to about 16 mg/kg.

72. The method of claim 71, wherein the anti-Galectin-9 antibody is administered to the subject at a dose of about 1 mg/kg, about 2 mg/kg, about 4 mg/kg, about 6 mg/kg, about 8 mg/kg, about 10 mg/kg, about 12 mg/kg, or about 16 mg/kg.

73. The method of claim 72, wherein the anti-Galectin-9 antibody is administered to the subject by intravenous infusion.

74. The method of claim 45, wherein the subject has undergone one or more prior anti-cancer therapies.

75. The method of claim 74, wherein the one or more prior anti-cancer therapies comprise chemotherapy, immunotherapy, radiation therapy, a therapy involving a biologic agent, or a combination thereof.

76. The method of claim 74, wherein the subject has progressed disease through the one or more prior anti-cancer therapies or is resistant to the one or more prior therapies.

77. The method of claim 45, wherein the subject is a human patient having an elevated level of Galectin-9 relative to a control value.

78. The method of claim 45, wherein the human patient has an elevated serum or plasma level of Galectin-9 relative to a control value.

79. The method of claim 45, wherein the human patient has cancer cells expressing Galectin-9.

80. The method of claim 45, wherein the human patient has immune cells expressing Galectin-9.

81. The method of claim 79, wherein the cancer cells are in tumor organoids derived from the human patient.

82. The method of claim 80, wherein the immune cells are in tumor organoids derived from the human patient.

83. The method of claim 45, further comprising monitoring occurrence of adverse effects in the subject.

84. The method of claim 83, further comprising reducing the dose of the anti-Galectin-9 antibody, optionally the dose of the checkpoint inhibitor, or both.
